SEQUENCE LISTING (129 sequences)

SEQ ID NO: 1 - DNA, 6000 bp, Artificial sequence
  C1orf158
cttactttgatggtgcaaaagcatttgtgggtaaaattgctggcacctgtgaacgaatcaaggccgtgccattaattatattagtatcctttattcttcactgccacacactcacagtttaaacaaaaaaattaagttcacttaagaatgtcctttgatgaagaagtaacagttatttgatcaaatcttgaccctgagcaaatgcctttttagcactctgtgtgatgaaatgggaaagagatacaaataaggcactgacgttgcacatccaagggtgagggctgtcttgagaaagagcatttgtgtggattattgagttgtgacctgaattaattagccaggcttttttttttttaaatgaaacaccatttttacttgacagaccatgattattcacagacaaaccatgattatttaaatttgatttttttttttttttttttttttgagacggagtctcactctgttgcctaggctggagctgtggtgcgatctcggctcacagcaaacttcgcctcccaggttcgagcaattctcctgtctcagcctcccgatgaaatgttctcttgatgtttccttgaaaatgaatgaggtaagtctgtcgcttcaaggaaaacaactgagattaattgttgccaataacaaaattttgagctttcaaactagaactagaattttggaaagcttgcgtccaccgctgtgagcttaacaacttcctaatagttcaatgcttttctgaccatatcagtaatgatgttagcaaatgtgattctttcttttttgagacagggtctcactttgttgcccaggctagagtgcagtggtgcaatcgctgcttactgtagccttgacctcctgggctcaggccatcctcctacctcactctcccagtagctgggactacaactgtgcatcaccacgcctggctaatttttgtatttgtttgtaaagatggagttttgtcatgttgcccaggctgatcttgaactcctgggctcaagtgatctgctcacctcagcctcccaaagtgctggtattacaaggtgtgagccactgtaaccagcagcaagtgtgctttttaaaaatattgtatagtttggaagagctacacagcttagtaaagcaatattttccagatgaccaccaaaatcaccaatgggcaaaaaatttattcaaattttttgttttggaaaatggatttaatctttattaaaaaattaattatgttaacatgaaatgagtttttttttgttatttggaaattaataaatatattttaaatttctgttttaatttctttctttctttttttgagacagggtctttctctgtcacccaggctggagtgcagtggcatgatcgtagctccctgtagcctcaaactcctgggctcaagggatcctcccatctcagcctccttagtagctgggactacaggtgtgcattaccacgcctggctattttttgtgtatatatttttttaagaaatggagcttcaccacgttgcccaggctggtcttgagctcctggggctcaagtaattctcctgccttggcctcccgaaatgctgggtttacacacatgagccactgtgcccagactgttttaatttctaatatggtaaatattgatgaatataaccagtaacatgttgacaaactcattttctgggaaaaaaaacccaacagcctaatatgtagcctttgccaattctgtggtgtaaatattcccactgcgactgatgtcaagctaccaatgtgacttcatagagcatgggattgggaagagatcgcaactggctctcatgagcaggtgctacctggctccagcaccactaatataatccacatttaaaaagccctctgagtcctcaataatttttatgaggttaagaggtcccaaggccaaaatgcttaagacccactgccctagagaaatggctatatttgagcaccaggatatacgtatcatgtgatctgagagaccaaaatagacgcccctgtatcaactaagaccctaaggctaaggaaacaaaagctacctacaggttgagggttcagagcttggctggcctgataattttttttttgagacaaagtcttgttctgtcacccaggctggagtgcagtggcaagatgatggctcactgcaacctctacctcttgggcttaaggaatcctcccacatcagcctcccgtgtagctggtaccacagtcatacaccaccacacctggctaatttttgtgtattttgtagagacagggtttcatcatgtggtccaggcttgtctcgaacacctgggctcaagcaatctacctgccttggcctcccaaagtgctgagattataggcatgagccaccgcgctcggtctcagcatggcaaatttctaatctcctgtggctataggaaaaaagacccttgctaaactccctaataatagggcccctcaggctgatttacaacctaggccactacaactctgattggacagaggactggccttacaaacattcttttctggcaagttattgcagacctaaagccagtttcagccagcttatagaggctgtgcacaaactctctttgtgtcctatatttcaccttttgacataaagaaccaaattccacctcatttaatattaaaacctggcccacactttgcaaaccggtatcaccaataaagctgtcctgctattcagccaccctggtggtctttcggatgacgatcatgtacaaaacagtcatagcagccatgtctatgtgcaacatgggtgaatcttaaacacattaacaatattggggaaaacaagccagacttgagtaaatacatacgataggattccatttatatgtagtccacaaacacgcaaagctaaacattattgtttaggaaagatgtatgttacatacatgtttttccattgtgtatgtgctcagttccactcataagtatgtatagcttccccccaaacctgctgaatatatataaacacaggccttgtgaagcatgaaacccaacctgtccttcctctcttggaagagagagtacctctgatccatgctggagactgtctctctgtgcagtttgcaaactgctatcgccattaaagctcttctttttactatttagccatgctggtggtctttccaatgactgtcatgtataaaacagtcacagcaaccatggttacatgcaaaatgcgtgaatcttaaaaatattaaccccactggggaaatcggactcattgcatactataggattgaaactatatgaaagtccccaaacaggcaaaactgaacattattgtttaagagcatactttggcagtaagcatataaagaaaagcaaggaagtgattgctatagaagtcaagagagtggtttcctttaggggaggaatgggttgtgagtgggaggggcatgtggagtactttgggcgtgctggcgatattttatttcttgaccagctcagtgttttttgtggggttttgctattccataattcatttatctgtacatttatttttaacgtactttttgatatttgtgttttatgcgacaataaaaggttttgaaaattgaattataccgcgctaggtgtggtggctcatgcctgtaatcccagcactttgggaggccgaggcggatggatcacgaggtcaggagattgagaccatcctggctaacacggtgaaacctcgtctctactaaaaatacaaaaaattagctgggcgtggtggcaggcgcctgtagtcccagctacttgggaggatgaggcaggagaatggcgtgaacctgggaggcagagcttgcagtgagccgagatcatcccactgcactccagcctgggcgacggagcgagactctgtctcaaaaaaaaaaaaaaattaaattataccgaacgcatgcctcctctgcatgttaattggaaggacacctcctcttcctagctccagccctgccccaactgtggtctctgctaaataaaggtttattctaacctgcagaacatctttcatggtcatttcctgcctcaggcttagtttcaagatggaacacatagtttcctggagttcctctatttttgtttgaaggccattggaaactttctctgaattgcctagcgaaatccagcctcttcacttttagcaagcaatactatagcacacagagttttgtttagttaaccacatgttataggcatcttttaaagtcagcctttaaaactcttgcagaggatttcatcatctggatgtattacagtttattgaacaagccctctgctgatggatatcttggtgctttttgactttttttttttcctaatgaatgagcaagacaaaggcccttatctcattggactgagctctgtggggatggtgcccaggtctaattgctaatgcattgcagctgtagctaagcacctggcatagaatgggaaatgattgtggaatgaatgaatgaagactgagcaaatgaagcatattataccagtgtgagcacaaagacagaattctttgtgcaagcccctacaaagtagcagataggaaatgaatggagcccactggtttaggtctgaaccagcgtggatttaaatcccatcacaactgcatgatgttaggcagctacttaatctctttgcttcagtgtcctcatctgtaaaatgggtataataatggcatctgcgtcagagggctgctgtgaggattaaacaggtgaataaatgtgcataaatgtctttgcttggagcaagcacatcaacattcattaaatagtagcagactttaactcaccccaaatgtgaaagtgtagcaaggatgatactagtattgtggtgagaacacagaaaccaggttcttctggcgactgcttttgtggtgtggtggttggtttgatttgtttctgtgaggtcaattttgcttctgcagaatcagctgttctcttacagagagtatttatactcagagtctgtcaccatggagacagtcaacagtagagaatccaagatagatcaactctccctaaaggctgacagtgaactcttggggccgttttattctctgaggttagcaaggagtcatctactagccattcaggaggccagctgggaagacaaaataggcaccccaaactcagcaacttcataacaccttcctctccccgcctgaagccttaaactgcatcaagtcaaagaaacctggggcaaatccttaacatgtttttgactgcagtaaatccacagccactctctactccgagctggcagattgagaccaagtattcaacgaaagtgctcactggaaattggatggaagagaggagaaaggtaaggaaacgagagaggttggagagagggctcagagggactgatggggagaggcaggagtgaagttcatcactattttcaaatggagggcagcagatgattgcatcttaaaaatgtggcattggggtctctgtgctctacaaaggatagttacattcaagcaatcatcaatattcacgcattttgcatggtttctatgactgtttcatacatgatggttatcgaaaagaccactgacactctcttacagatataacaggggcattgaaaatactgtaacagggtgtcaatatagtcgtgcctctcagcactcttctcagtgaagtgtttctaggcatctggatgttttctgtccttaattcctttgtaccttaatttgaagaacatttgctctcctcattctggcccttcccagaatatggtatctcctgaggccagcttgccactctctgtgctgatgtccagggatttgctgctgctctctggcacctttccacaatcctgccctactaatgcatttcaatttttacttttttttttttttgagacagagtctcgctctgttgcccaggctagagtgcagtggcatgatctcggttcactgcaaccttcacctcccaggctcaagcgattctcttgcctcagcctcccaagtagctgggactacaagtgc

SEQ ID NO: 2 - DNA, 6000 bp, Artificial sequence
  IGSF21
tattaatggaaagagggttctgaggccaaacaagtgtggacttcaacaaaataaaactggaaaatgaacatagacaagccaaacacaaaaaactgcaggacttctcataacctttcataggctcacgtgaactataaacttctaagtggagaacagctgagtttgtagcacttcttgaatatatttagccatgaaactctcttgcaagaaatgtttattacaaacctgcaaaacaaatggtcagtgagacccagtttggggagcaatggtcctcctgggtccatcccttttctctggtcccataatgctgaggccccttccccagcccacagctcgagattcccacgcacacctgctgacatcttctaccgggaagatgtgatggaacttgagagtccaggtggggctgaggttcattaaggatggagcattggacttaattccaagtggctgactccatatcaatttgggtcactggtgttaagatgtcactttcggttgcatttaatttaaacaaacaataaaaagctctgtcctgactgcgatggaggctggtggaggtttaattcccagcacagagaggcagaatgcaggataggaaagccggaggactgcaggagtgggttgatgagagagggagagaggaggatagagagggagagaaatgtggacccctggggcagggcctgcctggggaagtccacgctagatccctgtccccagaatccagtatcctctaccctggccaccttgggtaattattttcatttctctgagactcagtttccttatctgtaaaccagacataaatgaaattgccacagaggacttacaggaggattaaatgagataagagaggtggaacgcactggattcatgagtttttatatacaaatactggcttccaagatgtgtgtctgtgtatatgtgtgtggacacgtgtgtgtgttcctgtgtgtgtggacatgtgtgtgtgttcctgtatgtgtgtgcggacacgtgtgcgtgtgttcctgtatgtgagcgttcctgtatgtgtgtgtggacatctgtgtgtgttcctgtatgtgtgtgtggacacgtgtgtgtgtgttcctatatatgtgtgtggacacgtgttgtgtgttcctgtatgtgtgtggacacgtgttgtatgttcctgtatgtgtgtgtggacacgtgtgtgtgcgtgcctgtgtgtgaggacacgtgtctgtgtgtgtgtgcctgggtgtgtgtgtgtgcgcccctgtgtgtgtgtgtgtgggcatttgtgtgtgtgttcctgtatgtgcatgtggacatgtgtgtgtgtacacatgtgtgtgcctgtgtgtgtggacatttgtgtgtgtgtgcctgtgtgtgggcatttgtgggcatgtgtgtgtgcctgtgtgtatgtgtgtatgcttgtgtgtgcctgtgtttgtgtgtgtacacttctgcttgtgcctgtgttggtgtgtggacacttatgtgtgtggctgtatgtgtgtacacttgtgtgtgtctgtgtacacttgtgtgtgtgtgtgtacacttgtatgtgtgtgcctgtgtgtatgtgtggtgtactgtgtctaggtaaggtgtttgggaactctggttcttgagctcttcctaggttggaatctggtcctattcatagctgtattgtactgagaaagttgcttaacttctctgtgcctcagctttcccaactgtaagacaggactaatgatgggacccacctcatacattattgtgtggtgtcaatgaatttatatacattaaatactttcacagcacccggcacaaagaggcactttcataagtgcttcagactcttattattgaacctcactgggtgtcctgctgcaaaccagcagagcccattcccttgggagccaggttggggtaggcagtcatgtgctgcgtccctcccctttcctggcagggagggtggggactagaggtcacagaggggccctttgacctctggggctattccctggggtccgcggagtagaagtttgctttgtgctgtagtgcactctgttgggagagcctaactcagcaccatgaacagagggaggctgggggcaaacagccacacctccgccaaggactccagctcagccagtgtccgaggaagaggcctgtcctgccgtgactcatgggtctgagtgccaggactgcaaagtggaggccctgccgatccattaggagacaggagccaagggatgttaagcaaattaaaagccccagggctgtcccgcgtgaccttttcctttggctaaggcaccccaccctgtgccctctgctaactgtgcttctcagtgctccagagatgtctttctctgggacaaaggaaaggctttaaagctatatcttatgaaacaggagctggaggagtgggcttccctggaggggcctgtgaaggcagccgtattaaccactatggttaccatatggccatagacatgcacatcacagccaggcccctttgggcaaaaccaaactgcgcacctgagcagacacctcctctctgatgggcccagtcagaatatgtgatcctggcgctccaaggaggttcactgccacaggacgcctaccaagtgccagacttgtgcaaggagctgaggaacagagtggataagactgaatgacctaccctcattcattcattcattcattcattcattcacgcattcatccaactggtatttattgaggatccactatgtgtcaggcactggctaggcataataataattataatacagggtgatgttaaagagaattgaggtgattcagaagtcttggggctgaaggagatctccaacacaccagccccaatccttggtcctaggaagcaaccctcccttggctgttaggcctgctttcctcctcactagcatatcctcagatgccattctctagctccttctgctggctctcttttctagctggtgtgaaccatggctgggaagaaacatcactaaggggccctggaatcccacctgctccctggccctcatctccaagggctgggctgcagggagcgcagccaggaagccccaccaattcagggttcacctgaggaactgatgctgtccacctgcctagctgcacgccgatttgcagctggggccagagagtaggaatgcccacacagcgatgcttggcatttccctgcacaactcagaccagcacaaggaaccgcatgaacctgatgctgttccccagccagggagccccttccacagaggatttagaacctgggatggacttttgggtttgttgactttttcttgggtcagagtggggagggagggccagggaggaggcagtaagggactgtgccctgcagcctgagaagaaggtgagggggagaggtgacctcaagctgaccctcagcagcctggagaaggggagacctgggtggatgcgagtgagagggagcaaagaacagcagccaaagggagaagaaaggacctaagactccagtgttcatcaagtgttcaagccatatgcagtccaatcaacctggaaccaattagtctgattcacaaaaaaaaaaaaaaaaaaaaaaaaaaaaaatcccacttagggacaaactgtaatcaaacacctatttataagggtttattatgatcattattattattttactgagaggatcattctaaagcctgtcaggtgaacgtatgctttcagatcttcataaatgcaaatcgctccttctggggtgggtgagcaagctgagccgcagagctgccttgggaggcgatggggtgaggttccagagggcaggaggagggagcagttcactttcatggacatctctgcacaaagctcacccagaggcagcccagcaaccacccattgcccaccccactccctacactcctctcagagggctgctggagatggaactggctatagacccattgcccatgtaggtaaacgatgcaacagcctgactcagcaacctgccagagctagggaagctggagattctccatagaaggcctgggtaaagggacccacctgagttctgagcaggttttgcccctggtcgtgctggtttttctgggctttgccaatcttctgggtccctaccaatggtgaaattctatctcctgaacttcctcatctaaccacatccccagtccactccaagggctagagtacccctccccttcatcctcgcaggaggcctcaaagtccaccgccagggctgaggctcaatggtgtgtgtgtgtgtgtgtgtgtgtgtgtgtgtgtgctcgcgtgtgtgttggtgggagagtaatggtaacatacaagtcccaaacctgttcttctcggacctgggaagaaaagctgtcaggtctggcaaaaaggtggaaacttggtctctgccaggaggaaacaatcagttcctcacccttcctggctggatcctagcacatgggaaaaagacagaacacaccccatttccgtgggtccctgggggaaggagcagccgtaattggggaagtttcagaacatggaaaccccttaatcttgcccaatgagggcattcatcgttggcaaggtcagaactccagagccacaccctgcctgccctgctcccaggatggcatctttccctctcgggagggaggctgcctcctttcatcaggctgagtagcggggagggcgatggtaatcccggggataggaggggctaggtaaaggcggatccgatggagcatagcttccagggcggggtgttgggtcacctgggtaagggttaagaagctggaggcagcaagccagttttgagtgaggggggacctgagtgaggggagaggggaggttaggagggggtgagctctccttctccctgcaataaatcggcttagcggacgagagccgaacagcccagaaaggattaaagaaaagtctgtataatacgcggagagcgcggcgaggggagggcaaggagggcgggggggcggggagagggggagggacggagggagggcgagaggaggggggtgctcgcgccgccgggagaggcgagcgcgaggcagagagcgcgattcggctccaaactccggcgctgcagccgatcggactctgggccgcggtgggcaccgcgcgcagctagggagccgagaaccgcggcgagccccgaggacgcccagagcgcgagggtcgctgcgcctcgcagagccggagccgagtcgagccgggcgcccgggctgcctggccgcggcggcatgggggcgcccccgcggctctccgcgctgcccgccaccgcctcggccagtggccggaggcaggagcgcgtctgagcccatggcgaggggacccgccgccaccgcctccacccccgccgccccgccaccgccgccagctcccgggcaccatgcgaaccgccccgagcctccgccgctgcgtctgcctgctgctcgccgcgatcctggacctggcgcgcggtgagtgcgcgggcgcctggcgggagccgagcggtgaacgtgcgcggggacggggtgccgggggagggcgctggccggggtcgctccgagaggctcgggctacgagcaccggtcctgcccggggtctgtggagctggttggctcgatgagggagggaggacgcctcttggagagcgctcatggatttgtgccagggtgtgtgtgtgtgtgtaaattgtgtgtctttgttgtgtgtctgggtgagggtgtccgggaaggagctgtgtgggcagaaggtgcgggagtgtatttagagatgcaagtgtgtgtgtgcgtgtgtgtgtgatggtgtggggtctgcgtgtgagtgagcgagggtctggatgggtgttagagtgtctgtgtcagttacatggagaaggtgtgtgtgtgagagtgtgagcgaatgttggggggagggtgtgaacatgtgccaccttccctgtgagggtgtgaagtgtgtgagcttgtgtcactgtgggtgtgaggtgttagggggtgggtatgtgaggtttggcgtttcacgtgt

SEQ ID NO: 3 - DNA, 6000 bp, Artificial sequence
  HFE2
agagaaaaagaaagaaagaaaagaaaaaagaagagaaaaagaattacatgaattaaagactgtgtggtattggcagagggataaatatacagatcagtgaaacaatatagaaaacccagtagtagacccacacaaatatacccaactgatttttttttcttttttttccaatgagaaaatgctaaaacaagcaaaacttcatgaagggacatctcatcaaagacgatatacagatggcaaataagctcataaaaatattttctacatcattagccataagggaaatacaaattaaaacccaaattagatatcattacacacctatcagaatggctaaaaaattgttgacgacatcagccaagtgcagtggctcacacctataatcccaacactttggaagtctgagggggcagatcacttgaggtcaggagtttgagaccagcctggccaacatggtaaaaccccaccactactaaaaatacaaacaatagcctggcatggtggtgcacacctgtggtcccaactacttgggaggctgaggtgggaggatcccttgagcccaggaggtggaggttgcagtgagctgagatcacaccactgcactccagcctgggtgacagagtaagattctgtctcaaaaaaaaaagaatctttgttggtaaattccttcaggttttggtgtgtttgaaaatgttttacttatttttaattctggaataatattttagctgagtatggaagtctaggttggcagttactatctcttggcttatcagagccacaccacaactgtattctcaacaaaagagagatgacacaccactctgctaccacaaaaatgaccagataatcccctcttcagactaacatgagtaactaatgcttttttctctttaccaatttgggttataatcctcttgcttcctaggtaagaattatttagacacccaatcactgaaacgcccccacttcttaacagtattcaatcccaaattagtcctcattcagagttttagagactaggaaaaaacaaacaaacaacaaaatcagccctcactattctaaactgtagttcagaaatatccaacagaagctgaaactctataagaggttccttttaacctctccttagggaaatggtcccataattcctttggggcattctctcccttgctacagcaagccagtagatttaacttttttgactacagatttgtttccagtggtgtttagttaatgagctttgacaaagataatggtcaaatttctccaatatcaattgactatgtgaagaatatatatatatatacacacacacacacacacacacacatatttgtgtgtgtgtatgtaatatatacatatttatataagaacatattggcagggcgcagtggcttatgcctgtaatcccagcactttggaaggccaaggtgggcggatcacttgagcccaggagttcgagaccagcctggccaacatggcaaaaccccatctctactaaaaatacaaaattagctgagtgtgatgacacatgcctgtaatcctagctactcaggtggctgaggcatgagaatcacttgaacccaggtggcagaggctgcagtgagccgagatctctgggtgacagagggagaccctgtctcaaaagaaaaagaaaagaaagaacgaacccaggtcatttgtcctgtagagtattcacaaagtctgaatttgcacctttgagttaccacttaatgtctcctgtatttcctgtaaattagtagttagctctaatcagattcagactggtttgtttgtttgtttgtttgtttgtttgtttttggtatggctacttcatgcaaggtggagttatgtactctatgatcaggaaaacatgtttatctctgttttcatgatgaataacagccactgtttttttgtttacgtagagatgggctcttactatgttgtccaggctggttttgaactcctgggcttaagcaatcctcccacctcagcctcccaaaatgctgggattacaggtgtgagccactgtgccctgccacagtcattgttgaccattgcctagatatattaattcatttggatttacaaaatgttgatattctaattatattatcccttcttcaattatttactggaatgcttctataaagagaaatttccccctcatcttgagatacagttcccaaaagaaaaccaggataaatacttgattctttccaattaccagcagtttttaaaataatgagttgatctgccagcattctccaacaatgaccaatgggtttgtatttttaagtatcattgtgaattcatggatttaaacatattttatgaatttcaatccattgcagatagtatccattttgatacttaaattgccccatctttggccaatgggaactattctagttggctcctaagttcttttattacaatcctaacactctttgaaagcttccttgcctatcttggacaatacctgccccaaacctgaaatcagccacttatccaaggagttgtttgttggtcccttttaacagaaaatggtatttacatagcacaatttgagtactagaggtgtttatttttactggatcattgtttccaggcctttttagggtaaagctaggaaaattttaaggataaaataaaccatgagttcagagttatatttgcaatttaaattcagaattacggagttttctcttaacttcatcaatcgtaaatatgtatctctttattccaccccaaaaattctggttctcagagacactaacattattaatcatttgttttatctcataactaaaataatctcagaataacaataccaacactaacaccataatatggctatttaaaaatatttttgcatttattttctggcattatagtatatcccacttaggctgtcatagtcaaattattatgttttaaagtcacttgaatagtttggttagaagcattttacatttctataatgaaactgcttgtgatatggcctctaatggttgagaaatatttgtcatatatatatacctgagaagtatatatttgacaaaaatatttgtcatatatatacctgagaagagtgctatgagggcctctagactctgtattaaaatagagccaactggtaaagatggcttagtgattgtgttggttattactgagtgtcaatttgattggattgaaggatacaaagtattgatcctgggtgtgtctgtgagggtgttgccaaaagaaattaacatttgagtcagtgggctgggaaaggcagatccacccttaatctgggtgagcacaatctaattcactgccagcacagctagaataaaaagcaggcagaaaaatatgaaaggagagactggcctagcctcccagcctacatatttctcccatgctggatgcttcctgcccttgaacatcagactccaagttcttcaattttgagactgagactggctctccttgcccctcaagcttgcagacagcctactgtgggaccctgtgatcgtgtaagttaatacttaataaattcccctttatttatatatctacctatatagatatccatatctatatagatattaataaatctagagagacagaaagcagactggtgatggccagtctagatggctagatagatagacatggatatagatatagatctctatatagatagaggtagatacagatatagatatatgccctattagttctgttcctctagagaaccctaatacagtgaccgtatttggaatcggtccttctgttaatttcacttggcaagtactaaaagatgatgatctcagatatacctatggctgcaaaaacatgacatggctaaatcccttggttgcagtatctcttttcttttttaaggggggtgggggggcgggtctcactgttgcccaggctggagtgcaatggcgttatcatagctcactgcagcctcaaactcctgcgctcaagtgaccctcctgcctcagctcccaaagtgctgagattttgcaatatttatggtcacaagattatgttattccataaaagtatctttctgaggctaggcatgttggttcacacttgtaatcccagcactctgagaggctgagatggaaggattcattgaggcaaggagttcaagaccagcctggtcaacatagtgagacctcatctcggaaggaaggaaggaaggagggagggaggaagggagggagtgaaggaaggaaggaaggaaggaaggaaggaaggaaggaaggaaggaaggaaggaaaagtatatttttgaatctttttctatttctccaactctttctttagaagaattctatttccattctttcttcacctctttgcctttgttagccttctctccaagcaaatcgggagcctttattttttgtgtattcatgagggagaggaagatgaattgctgtacaaactaaagtaatgaaaatggagtaggtaggaggatagacagctgcaaggatctgagctggatagactgaacaaaccctcatcctaagcaactcacagctcagatttcttctctggacagctggcttttttcgtccttctgaaatactctgcaaagataggagaggggctatgaactacctctgctatggatcttattcaaagtcagctacctcctagatactatctgtagaacctaaatgtaatattcagcatagcagggatgaacatggtaaatgaaaggtatccaattgcccactgtaatttttaaaggccaggagctcaacattattgaaaatgctggagggctgcctggagtaggcagtgaccacagagtcacacaagctggaattggatatccaacttgtctgtcatatttctctcctccctccctgacttggcactcaatactccatattctttctaatcctctaaccctccccactcccccaactcccacaccctacccccaccaacgttcctggaattttggacttagctatttttaaaaccgtcaactcagtagccacctccctccctgctcagctgtccagtactctggccagccatatactcccccttccccccataccaaaccttctctggttccctgacctcagtgagacagcagccggcctggggacctgggggagacacggaggaccccctggctggagctgacccacagagtagggaatcatggctggagaattggatagcagagtaatgtttgacctctggaaacagtaagtcaaaatgaaattgcaattcctttaataagcttttatattgaagttagacttttataaaattacaaacacctacttggatgtctctcgtccaaatgctgggatctctccctaccaaggtgccccaatctccatttctctttctgtcttatttctttctggcctctggcctctagctttttgaagtttaattctctgtctctcctctggcagtcttagccctctctttaccttattacctcaagactcctgatgaagttttagaaggagttccctacgtcctctattctgtagttttcttaccaaggccaaatatgacctcagatgatgagtcactgatacccttctatcctgcccccacttagcaatgcccttcacattgagattccaagcatgggggctgctccctgtaaatgatttctccccacaactctagtccctccattctattctccctcttgcaggactcttcccccaatcatatccttacccataagataggggagttaggcaggagggatttagcccctctccaactcctgtcatcataaaagactgagaacttcagaatttgaaaagaagagattaatggaaggagtgatatttgggaaaatacaagaactgttgacttagaaaaaacaaatattgatttgcatgtttggtttgcatcccattattccatgagagagggagattaaaattgcagctctctagagctgatgaaaagagattggtttccttttcatttgaatactgatatt

SEQ ID NO: 4 - DNA, 6000 bp, Artificial sequence
  CRNN
ccttccagtttgcaggttctaggagttctgcctatggattgaacacctagatataggatatgcagagtccctactaaatggcagattccagctcttctggcaaaaccaagaatactaacaatcatgttagccatgtgcctgctgcctagatcagaactcagagaaactgcagggccaacacaacctgtctgttcagggattaggcccagatagcctgagagatattcactaagccactggaaattgtgtcaacaggtgcgtctccaatgtctgcttaatcctccctggcatttccagggcaaaacttgagcatctgggcttccgggattttatgatcaggggctatgtggagcgggtttgaggaaagagattccaagttaggcagagagaaagtaagaaggcccagaacttctcactgttcttttttcctctaagaaccattcccccacaaccctgtctttcagtaaggatacgtgggcaacatgaaccagcaaattctctcataacccaagcaactctagaaaacatctctccagctttcagatttggttttgttcttttctgaaggtaaagaccaagatcatggaatttgctcatctgctactttttgagagagatgtgagtggccaccctgtagccattcattgtcccatattaccgttgtgtgctcctgggtgaaggtgaagatgtctggtgagcagcattctttaagggttgggtttttggctgcatttgtaatggcagaaatttaaaggcagccatgtcagggttaacagttacctgccacctgacccaagaggatccatgtagcttaccatggtgtctccctgtccccttcatccaccagccaatcaggacctgacagcagacactgatgaagctgcactggaagagacacttcattagacagacggagtttagcctgctgagcagtctgcctcggcctctgtgtgtgtatgtgtgtgtgtgtgtgtgtgtgtgtgtgtgcgcgcgcgcgtgcgcgcgagtgtgtgtatgcgcgcgcgagtgtgtgtatgtgtgtgtgtggtaagtacatagctgtttggggcagtcaggagataacgatcatgatgtaggactggagggaacccaaagaaaagcaccacctgcatgaaagcccagctgttccccctggctgaacttatagaggcttttgccaaacattctggattttgccactgaacaaaggggaagggggaagaaggagaactgtcagtatgaagagagattatttccttgggctttgtccccggcatctcacagggcctctggatttgagaacttgccctgtttgttactctctgtggtcccatagctagttcacgtagtgtttaagctggaacataccatgttgagctgggtttaagtcaaagggaattttccagacttcagataagaaacttcagccaagatgcaaagcagagaggttaagatgctgggctctgaagttgaacaggtttgggttcaaatcctgcctttaccatttattttctgtctttggaaaattaagttagttaatgataatttcttcatctatgaaattgggataatatctttgctaccatagggttgttgtgagggttaaataaaatgatatgtgtaagtttttagcacagtgtctgtacatagtaggcacttagcaaataaaataaagtaaaataaaactagcaaaccaaaacaagcacaggtagggggtgttgctgacatagaccctgatctctcatattcctgagcagtgattctttaccccagaccttgtgatatttgacaatattttttagttagcattctaaaaatttctacttttcattttaaaataactatttttggtgtgtaaagcctgtttgcccaattgggctaattttctggaaagcaatcttaatttatagcccactaagtgtggcaaatactgcttgtatcttgtagaaataaatcaagtagaggtcagcaatacattgttgagtaagtgtataagaaggagtcaaagtgcaaaactgggttttcattgctgagttgctgatccagcacctggtctcactgccctccagcatacccgtaaaatgtaactgctaagtagactcactaatgtcaacttaaataataaccacagtgaatctctcttaaaaaaaaagttacctatttgagaatagggcattgcaatgggaatacatgtgccatagtaaactacgtgcatattcaggaggtaaaggaaaacaaaagttcttacaggaaaaacaatgaaaattacataattttattgaaatgtgtattcttggctacaaagatcaataacaatggtgatgctaatatgaagttggacaggcagctgctggactgatgtcctcacagaagtgtttgttgtgtaaggctattatggcctttgtgtaaggttgtggtttttgcagtcttttgtgatagttgtgttatcaggtgtacaagcatgagaactctctcttcgcagccttccttagctctatatttgtcaaggatttttttgaagacaagtgactccattttgattctgacaacttgcacactaacttataacatctcctcaccaactttataaactaacaaacttacacagtcaattacaggctcagtcccaatctctgccaactcatctcccccagccccacctgcacacttcaacccacctccactggcccagcacacacatacagttctttaacctctacttctatggtgccccagctcctcacagctcagtcctgccccaggcacacataaagacctattaggctttggaggcaggcagacctaagttcaaattcaggtctaacttcctagctatgtgaccttaggtagtttacttaagtttacttacttactctctgtgccttggtttcttcatctataaattgggttaataatacctaccaaatattcatcttctagatacagcctctggcttgttacttccctaacttaccctcagttcccaaacctttctggaagttctaagccccatcagaaaaagcttcaaacaccaccacagaagaaggtctaatcggctctccctcttgttctccaacttcaccctctcatactggccttcttctaactctgatcaggcagaagcaacctcagccccctcttgctcccaaaggttgaagcccctactctgcttttccctggctggtatgcctaccccaccttcaccccaggactggagccaacctgtccccatgtagacagatctctccaaacacaaagcctgcatcctgccctcctgcagtctggaactccccagtgctctgtgccctgaggggaagtgctggaggctgtgctgttgctacagggctgccctcaatacaccagtctcttcaaccaaggcccttaccatgccttcctatcctgttgttccctttcctgtctgttgcatgctgatctataagtgaggatggtaaagatggctttcccttccagagtcactcaggaagctacacagtatatattatctgcaaagtgccactcaagagcactctttgggacttggcttctgagctcagaaaacttcctcctcaggaatggttcttcatgcattgaggataagtgtgatgttcataaggtgccaaaactcaatgagagaagaataaatggcagcatggtgcaacagagagaacacaggcctggagtctgaggggctctagtccagcaccgtctccgcttcacagagtggctacttctctgaggatttctcagtgttctcatttatgaattgggcttagccataccacctcagaggattgctagggagatcaaataagatgagatggtaatgaaaatggaataaaatcaaatgaaatgaaatggcaataccattatcattaacctcttggggacttacccctgggatggccaggctatagacttcatgagagttgaaactgctgccatcgtattcaacactgtattcctagggccttagccctctgcctgaaatggagcaagctttccataaatatttgctgattagccaccagttgagtttcctgtccttgcaatgaggagttaccacatgatcatggtaagccttttttctcatcagctacaaaatgctacctacccatagcatggggtggggaaggtattaactttttttgttttaattaaaaatgagaccaactttaaagagatgaaactggctttcttgtgtctcatacactaggtgtgaaaggcacttacaaaacaagaattcaaaaaatgttctaagtaataacagttctaagtaacatcaccacaaaaacatgtgtgccctctcagagtggctactttgtaaaagttaacctcaatagatatattcttgaacatttatattaaaaaaggaaacagtggccaggcacggtggctcacacctataatcccagcactttgggaggcagaggcaggtgaatcatgaggtcaggagttcaagatcagcctgttcaacatggtgaaaccctgtctctactaaaaatacaaaaattaactgggcatggtggcaggcacctgtaatcccagctactcaggagtctgaggcagggaattgcttgaacccaggaggcggaggttgcagtgagccaagatcgcaccactgcactccagcctgggcgacagagcaggactccatctcaaaaaaaaaaaaaaaaaaaaaaggaaagagtcccataactttgtaggctcatagagacaaagaatgttcaccaggaccccagcctgtaccaagcactgctgagcccatcacaatggaaagaagcttccctgtcaagaggactcagctacagaaggtaccaaatgtggtaggaggggcctgttaattagaccaaggcagtcacacatcagcaggtaaaacagagacaagaggaggtgtggctgggctgggctggatcttggatgaatcaagccttcccatagggcaggatatcctgtctaaaacaagagccttggttaaaacccctataaaaggttctcatcacactgacctggtactcctcacaccacttaacagccacttgtttcatcccacctgggcattaggtaagtcccctcataagaaacctctttctcattctcagtgtcttggtgatctgagctcataaaactggggcagtcaggtatggactatgcatccttcagagctagctgtgagcactgggcaaaccaacgctaccgttgggaaacatgctctcctgaagcaatcaggctttctcctcctccctgaggctggcctgggagcagctcctctcactgggaaactgtgtgggcagcggctatggggccacccatgtgccttcctggatcagcaaaggtttcttttttctaaggctctggaagcttctttgcagtgctgagagtctatgggatcagaatcagtttacttatgccaacctagacaataagatcaaactgtgtcatggatgaaggggtttacatgattcccctctcctacaccagggtgatatttaggcaaaatatgtgtagatttttctaaggaatctaaaatgtaactaaaaggtcatcttattattttattatctaaaggtcagtggttaaagtctgctacatggttttaaaaaaaaagaaagatatttttcatctatgttgaggaaaacatccccagttttttaccttgatgaaaagtttgcctgaaattgttggttaccaggtcctagaaagggtttctcctgaacagcccaccttttgctatgacttactgagtcctcatggccacactaatctgctttttctagaactcaagtctccttccttccttttttctctttcttctcctacctatatctgcctcgtcccatcctctctctggctttccagctgctacaggctccatctccccttgcatttgagacttgtcatctttgataccatctcctcctttgggtctctccaaggcttctgcttaatgaatcttcaagtctcttttccttttgctcatgcaaccaaacccaggcctcacct

SEQ ID NO: 5 - DNA, 6000 bp, Artificial sequence
  CACYBP
ggatccttagttcaaatgagggacaaagtttctcagtcagccccacttcctttctttctaactcctacctttcccttgcagaggaggtagtagagattctggaattgtctatttttatgaattccattattttgtccatggcatctctaatgaaaacaggttctagaataaaggagttgattagtctgaacagtactaattaactacaaaataaacgttagtgatcagcctcttcctctataaacaatgaccaattagacgtttccgtaattccatgtattatgtatagtacactctataaatgtaaatgtaatgcttgtctaaaaagtgcaatttattgtacattgtcccaacaaatgtttacttttataatcgttatgaacttgaattggattagtatcttgtttttatgtgtgaatgaagccttgtgaaataaacaaatgcaactgagaaggtaacaaggtgactgtttttgtgagccagtgatgttttcaatgctttgtgttgcccctttggccccattaagcagtaataaacatttgttctgaagtccatgtatgtcttttttatttttttagttgactttattctgactcatttgaacccaatgtttatgtaacacttcttacacctgaccccagactccagtcaacgtagaaaacacacagtatataccctgcaaaatgataccctgtgcagcaccaccacaaagtgcttcattttcctctctactgaggttccttgattccacgtaacagaaacccttgcaagctagcttaataatagtaattttaagaggcaattaattaaaaggaaataggatatctgtatcataggtcccaaaggccaaacacttgggcctgaactggcactaggacaaggggctggaatgccatcaggactctgaaagcaactgttcccaaaattgtgcttctgcttgtccttccagatgacttacctgcttcattcttctctctgaaaacaggctttctctgcttctaagtacacagctaccaagtttacatatcctctcttcaaactaccagcagaagactaccatctcaattgttccaattacaaattctggaaaaaatatgactggctaatcctgggtcaggtagctgctgttagtcctttaaggtacagcaaggtaggagatactataaaattcaaactaaacttcagaggcacttatgaaagtggagatagtgtgcagagaatcccaatcatatctcatttgcatttgcattttgtgctgggaatccatggaacccaagtgaaatctcaagagatttgtcagctcctcttggaattcagcatgtattaaaaaataatcaaagactgtaagattacagcttttggcccaaattcagaccacagacatctttcatttggccatgtggtattttttgtttgtttgttttctgagatggtcttgctctgtcacccaggctggagtgcagtggcgtgatctcagctcacttcaacctccacctcccaggctcaagtgcctcccacctcagcctcccaagtagctgggactacaggcatacaccaccatgcctggctgatatttctattttttatagagacaaggttttgccacatggcaaggctggtctcaaactcctgagcccaagcaatccactcgcctcagcctcccaaagtgctgggattgcaggcatgagccaccatgcctggcccccatctggtgttttaaacaatgtgaaattttacataaaaagtaaaacaatcaaaattactcagaagtgctcacttcaacagcacatacactaaaactgtaatctacagagaagactgttgtgtcccctgcacaagaataacacacaaattcattaagcattccatattttgcacagtccccagaaggtcatttatctgctgacaagcccgaagggaacagtatgagtcatagcaaaaaaaaaattaaaaatatccattgaatttggcagttaggaaatcatgagtaagcttgacaggtacattaactgggggaagcagtaaaggctgcctgcaataggacaactgaatgagaaataatttcattttgaaaggaaaattattccaaactttggaaacaaggcaaaaaggtgagtggcatgtcctttcagtgtcaaaccaaggactaggggattgtactttgttcatttcatcgttcactattgatttaaaagtcctatactgaatctatacattaacttgtagatttttgtccagtaagaatgcaaatccctacttcctttccagtggaacagataacaccaaaagttatagtttattaccctgtgagacattaacaggtttatatctaaccttgccaccttatactaacattacattgttaaattgcctataaatacctggctactcaaatacactctcagtaactaataagtcgaataaagtatttgtcacatgtccatttatatttgcataatattgtctggaaaagcctcccttagtgttacttctgaaagacctaaatagagtgaagaaacgatctgtgtgatgacctagaggaagaggattacaggtagagagaaagctgggtacagacttggaggcaagagcaaacccagtgggttcaatgaataagtaaagtgccagactgtttaaatcagggagcggggaaggaagagtggtgagagatgggttctgacatgaagctcataaaggtgagataagatacgtaaaacactcaagttcacattaaggagtttgtactttattctaagcctaataggaagctgttagaggtttttaagcaaagggatataatgatctgaaaagatatctcgagctgctctgtggaaaactaactcttaaggcacaatggggaagcagagaaatcagttaggaagataaatttcagacaaaataatgatgacttggacctgggtgcgtagggtaagaaatatcttttccttctacccattttaggctcactggctggggatcctgtaacaaaagacaaattaacaagagaaaagcataaatattaatgttagttttacataacatggaaacttcataaagaaatgaagacccgaataaatagttaaccttagtgttgtttagagtaggtttgaagaagaatggagaattgtgggaaaatgtgatggggctaaaagactgtgatctaagggtaataaactgggggaaacttagcaaggcctgatgttcatattcgtctctacgtccctgtgttttcagagataaagatgttacttttattccaggtatagacagggcaattctcacatgagggtattacgtcctgcttcagagcagaaaggtgcaagaaagttagagacattcctgcatatgttctttctcaaattccttcagttcaaagtattcaatatgtctaggtgccatatttttgggtagcatgtcccaaaccccgtcaggtggtagaagcagaggttgtgagaactcgttaaattcgctacatattttgaagattaagccagtaaggcttgttatataagatgtaaggcctgagatagaagactcaggtatgagtccttagcctgagcaattaagagaacgaggtaactgttcactgcattaggtccatgggatatttgtttttatttaattatgtacttaataattccaggctattggttggacacttaaaaaaatttatacgataaaatacacagctcttaggagttcatcccatgaactttgacaattctatgtatcggtgtaatatttccattacctgggcaaattccccttcgccgggccttaagcataatctacagaaaaaactacgtacataaatatacgtttacaactcaatgagatttctaaagtgaacccacctatctataatcagtgtccaaatcaagaaacattacctgaacctcaaaagccccctggtggctctttcatgtcattgtcctccccactatgctcattaacaccatgggctagtttttaaatggaaccacgcaatatgaactcttccatgtctggcttctttccctcaacttcatttttttgtgtgaaattcacgcatgttgcgtttcggtaatttatttttgtactgtacttccgattctttactacagataatggtcagcctccattccgctaacagctttttttcctctccgagttgctgattctaattgctgccttggacgatctataaagctgagtgcgcgctatgtgacctctcaggggtcgctgccttggacgatctgtaaagctgagtgcgcgctatgtgacctctcaggggttgtttccaaccgtgttgttgacatcttgagcctgccaaggactagaataatctgaaaactaggcttctctgggggtctcactgagtgacagggttagaaccagaagagaacatcgtctccagaagacatttcaccattttctttgatggtaaacaggctcacttataccgaatccaaacccaggcgagaactacggactcttgaaatggtcggtgaaaggggcgaaagcaccaggaaatcgtgcttcaacagtccatgactgaaaggagggcctgaaactgtggccataggcgggcccttttgttagggccttgacctgggcttccgctacagggcccggtcacgaggccaacgtagctccacctctacggcggccagtgatgacgccaccacgtcggaactgttagaccgcggtgacgtctccaccgcgccaaactcactgaaaatcaaaccgctaccattaggagccctccacgcttaacatatccgttctttctcgtttgaaagtaaccaggctgctcctccccatttttcgccttcttctcgcggaggctgagagactaaccttacacaacatggcggcctggtgtgtctggtgtcctagagcggacgaaagcaggtgactctctagtcaacttccgacttggactccgaagatcggtacgttatttccggggctgggttaacgcagcggttccgagctgcgactgcgcagcgtggcccagcgcggtcaaattataatacataaaagttgtcagggcggagagcaagacattactcttctcggattgccggttcgctcgcgagacttgagcgttgctaggagattcggcaggcgggcggagccagactcggcggggcggggaggggtggggctaggctcggcgaggcgaggaagggtgggtggagccaggcttggcgggctgtgcgtgctcgcggtgggcggtggcggcggctgcctcgcgaaggttcgagatccgtcgcgtgcgggaggcgggccgcgatcttgcgcagggtcggtgtgggcgcaggctgcagcgccgcgactcgtgcgggtaggcgtctgcgctcggtttgagggctcggcgcggggtttcctgttcctccttctgcgcggctgcagctcgggacttcggcctgacccagcccccatggcttcagaagaggtaagtggtccggccccatattccttatgccccccggctggagctgcagcgccagcctcccgccctaccgccgtttccgtgggctgagccgccctgcggccacccggtcccgcgccagtcagtgcgccgccttcccgggggacacctcactcgccccttgctgcgccgtcggctccccagcgcttccactcgacctcgcaccccactcgcctgctgggctcgagcgggggtgtgcggcgattatccgtgcaggcggtgcggggagtgggtctgggagagcggccctttgcgcgtgttcctcaggccctttctgccctggtttcccagccagtggacaggaagcttcattcaagcaaagctgggtgcaaacatgagtgtcgttcttggtagagggcggttggaaggtgagttctcagtgctagcacttgaattctcctagtcaggttttctctacacacgaggagctgtgttactctgggcaagttgtttagcttctctgtgtcgcagtagcatccatttcatagggttgttaaaaaatatgatttctaggtgtttaagtc

SEQ ID NO: 6 - DNA, 6000 bp, Artificial sequence
  OR2L13
agccgggtttggtagtggtgcctgtattccaagctactcggaagctgaggctcgagaattgcttgactccaggaggctgaggttgcagtgagctgagatcacaccactacactccagcatgggcaacagagcaagactctgtctcaaaaacaaataaacaaaacaaaacaacaacataaaaaacaggcaggactaattacacatacatttataaacttcattgaggtgcttatcttttctaactcttctaggtcatagcacacaactgttgttattcaaggaaattaaaataggattaagtgatctgtttatgaagaatctcaatattcacaaaaattaaaataatcatttatatattgtcggtgacatacttctgcatttattttacaaaagggaaaatctaagctcattttattcaaacaagtagttgttaataagatggaaaataattttttaatgtttgttatttgttttgatgatcataaagcatcttgaaagacagaaacctggactaatgtatttataaattttataatttacgtattaatatgtgaaagtatatatgtatgtatttatgcatattgagggaaatgttatacatacagaaggtgcacaaataactcttatgtaaacatttaagtaaccactatcaaaatataaataaatgatatgaacacctactatcccacatatagaattaatacattatacttcaatttcaacaagttaaccaaacacgtaaatatgaaatacaaccttaaagacacattgttgatgttgactagatgatacttctattttttacatttaaaatatagtgtttcaaattataaacatattgagttttgtgacaatatttcattacttattattttggaaagtcttagaattatacattttattttctataagtttaatctaaataataaatgcaatagacagaaagacaagttccacagttcattcatttcattcaaatattcaacattcttgagcaactgctatgcactaggctctgttccaggcagttggttataccagtgatcagaacaaggatctctcattgtgaagtaaaaattctggcatagaatgaaaataaacagtaaatataaccagtgtgtttattatttaatgtactagaaggtgaaaagtgacatgcaaacatgtgaaaatggagcagagcaaggaggaccaggaaattagggtttaagtgggagccctacaattttttaaagggtgtgcaaggataggcctccttgggaaagtgacatttgagaaaagagtagatttgagtaaattatgtggaattaattaagattatgagactcaagatggagccaagggcccactcaatatactgaatatcatcgattatttgaagaaagaaatgcccagcttgtaaaacatgcccactccaaaaactggctaagagtattttgatgttgattacaaaacatgcatcactaactctcattttattatgcaggtatattattgttagattaagatttacactaatattttttctttattattaattcattatttctttttaggttaaaaaatagagaattcatgatgggccatcagaatcacactttcagcagtgatttcatacttttgggattgttctcttcttccccaacaagtgtggtcttcttcttagttttatttgtcattttcattatgagtgtaacagaaaatacgctcatgatcctcctcattcgcagtgactcccgactccacactccaatgtattttctgctcagccatctctccttaatggatatcttgcatgtttccaacatcgttcccaaaatggtcactaactttctgtcaggcagcagaactatttcatttgcaggttgtgggttccaggtatttctgtccctcaccctcctgggtggtgagtgccttctcctggctgcaatgtcctgtgatcgctatgtggctatctgtcacccgctgcgctatccgattcttatgaaggagtatgccagcgctctcatggctggaggctcctggctcattggggttttcaactccacagtccacacagcttatgcactgcagtttcccttctgtggctctagggcaattgatcacttcttctgtgaagtccctgccatgttgaagttgtcctgtgcagacacaacacgctatgaacgaggggtttgtgtaagtgctgtgatcttcctgctgatccctttctccttgatctctgcttcttatggccaaattattcttactgtcctccagatgaaatcatcagaggcaaggaaaaagtcattttccacttgttccttccacatgattgtggtcacgatgtactatgggccatttatttttacatatatgagacctaaatcataccacactccaggccaggataagttcctggcaatattctatacgatcctcacacccacactcaaccctttcatctacagctttaggaataaagatgttctggcggtgatgaaaaatatgctcaaaagtaactttctgcacaaaaaaatgaataggaaaattcctgaatgtgtgttctgtctatttctatgttaaatgcctgaaggatactcatgagaggtttcctagaaagaaatcaaagcttctatcttaccacatataagaagtgaatatttcagaaacattgttaataataaacaataatatgtgtttgtgttgtaaacacgtacctctaaaaatgtagtgttccttctgtggtaccaattataatcatgcaacagttacaggaagtagaagttacccaaggcgtcctattccctaacaccaaaattgtaagacttatgagaatatccctaaaaatacagtcacacatccattgtataaaagacaaatccatgtttatttttataaaactttgttaaattatattgctaacaatcacttatcaaaaattcacaaattccatatgaaatcattattctttgcctggtttatcaacacctttatttagtaaaattttacagatacacatatatatgcacacacacatatatatgtacacacatatatacagatataagttgtgttaaaattgaattactcatcctatgctagaagcaactatacaatattagataggaatatcataaaaattgccttatttcatttatacatacaggatgatgttttacaaactcttctagcaatttatcctaatagttatttcaaagaagataataaatatttctattgagaattcatttaatttttttccttttttttttttttttttttacaaacactaagacacactttgtaagtttaaaatgtatgggccaggcgcggtggatcacgcctgtaatcccaacactttcggaggcctaggtgggcggatcacgaggtcagcagatcgagaccatcctgactaacacggtgaaaccccgtctctactaaaaatacaaaaaaattagccgggcgtggtggcgggcgcctgtagtcccagctactcgggaggctgaggcaggagaatggcgggaacccgggaggcggagcttgcagtgagccaagatggcgccaccgcactccagcctgggccacagagctagactccatctcaaaaaaacaaacaaaaaacaaaacaaaaacaaaaacaaaaaacacgtatgaacagcttgagtcaaatctgccttctggcagctgggcaccaagtctgctcccccgcaggctcctgccttccttcacattgcactgctcattgtgtggttctggtcctgggacctggtggtaggggctggagaatggggattgggcagcccagttccgctctcctcatgcagtgtcctgcgtctgtcatctgcttgggttgtggcttgtgtggaaggaccacgactgaggttgccaggccagcaaagacggggtgcagaagagtctcagccagagtgatgtgctgggcagcgtccaattttaccaccctccgcatcaggtcaaacagctgcacgtgctccagagagtcttggagcatgtcactcttcagacgtttgcagttcttcacatatgggctgtcagagctgttctcatcccaaactaggccccatttgtagaaatatttctgcttcttggtacaggagatcacgcgtaatgggatgagccgtagggtcttcaacatcaccaggcgctcttggttttcacaggtctggaagagtgtgaaacctggtagtacttaaagagaaggtagcccatgccctggacatggcaggggcgtgtccagcccagatcaaggatcagttcaggcaggcgacagtgaccgtggtcaccatggcgatgtggtgcccacggtcagaggtggcattggggaagtcagccactcggacgctggtgctcctcactgacttctcatcgctcctgtgctcgctgtagacggtgtttaactcagaatccacatgcaggatgttctcggttttcaggtgtgcgtgggtcagccggttctcgtgcagaaatctaagggtgtggcagagctggtgggccatgtgccagacatgtgggaggggaatggctggatgttattctccttcgggaactcaagggttttcctgcccaggagctcaaaggtgatatgcatggaccgcggagggtgaaccagtcacaccccaagacgcacagcagctggttctcttagtccttctcgtttatttttttcgagagcgttgagttttggcggtgccgcctcccggtgcttgcccacattgcagatgaccctccggcaacccgagacgtccttctggcagggtccaagcactcacccccttgccacaggcgccttggcccaggttcccacttgtctttattgctcttggggccaatcaccgacccggccaccaggtggcgcctgtttatcatcttccgcacctgcgccctgggctcactcgctggggctgccggaggacgcgctccttcaggacccgctgcggccaccaggtggagcctctttatcatcttccgcacccgagccccgggctcactcgctggggctgccggaggacgcgctgctgcgagaccagccgcggcgtctttggcagtagtgggcgtgcttgcgggtccaggagggcccctctcccgcgaccgccgaccacgatgagagcgtgaagaccctctcgaaaggaagggctctgctctacacactggtgtctcctgcggaagggcagctgggcacgccttccagaccgaggtatcagacgagaggccccctggggacggggcggttaccgcagtctcccttcttgctcccgactgtcagccctcctcccctcccatcagcagctcagggatggggctggctctggggcctcctcttccatcaacccctcagctacggggctggttcaggggactcctcccctcccatcagcagctcagggatggggctggctctggggcctcctcttccatcaacccctcagctacggggctggttcaggggactcctcccctcccatcagcagctcagggatggggctggctctggggcctcctctcccaggctcagctgccgctgggacccactcccctgagcgcctctcccctaagtaatgttattcatagcaaaaactagaaaaacacattttaatgaaataaatgttcaggttaattttattatagcatgccaactaaatgttttatactatagaaataaatatttaacaaaatatgaagatatatatgaaactttaatgaaaaatgattagcctttcacaattggaaaattaaaaacaatagtaataaatgttaaccatattttggtgaattggacatcataacatattgtaggcatacatgaaatatggtacaaacattttaaaaagaacagtgtggcaatatatattaaggaccttaaataaactatgaaattatcttctaataatacaactatttaatgttcataatttagacataaaataagcaattattatatattaataacattttttccctgacaattgagtttttttacttgattccagtcctgtaacagtc

SEQ ID NO: 7 - DNA, 6000 bp, Artificial sequence
  CACNB2
atggatttgcttgcatctttatatgcttttaaatagcagtctgtaattcagggcataggtttacaaaaagcaaacatgtaacttcatggaatgcatcataccaagtcaggccagtggccaatacagctatatatccttatgaaaatgctattaatagtggtggaaattcaggactttcagtgaggtgtaaaatgatttactgagtaaaatgagaaatcatacatgtgtatctgtcctaaaaagctctttctttaaggtgttaaatgtagtcacaaatctgcaagcaggagaggaagaataaagcacttgtaatcactgtttcttccacttacagaaaaaataatcttttaaaacgtctggcttcattagccagtgtctcttaccagcccctgttcctatttcagagcattagtaattcccagaatatgtctgtccattacaacaactaatgagcacaaggaagctaacatacagcttgaataaattagtcatctgcatttgaatgcgtcgatggcacatccattattgctggagtctcttgcatctgcttaacttttaacatgaaaagaattaggctttacttaccaaatttttgctttcctctgtggcttttaaatattgcgcaagtcaaagaatcttaaaaacaagatcaaccgtgattgaaggcatctatacattttccaagtctttttattctgtttattctgattttgctccctttttcaggagtaacattttgttactctgtgtttgaaatcttttacaagccagacaagagtctgaatgggtacattgtattggattagtgttcaatgcatgctaattaaactagtttgagttataaaggaagaagtagtataatgttctcatgggagtcaggcttaggtaaatgttgtcattgtacgttaaaactggcaaagttgtatgctgtgttttaagacgcaagaagtcattcaacattcatgcattcatttagacaacaaatgttttcacgttctaactaagtggcagacaccatgctgggtggtgctggggatactaagatgaaaaagacgccatcactgccctcaaggaaccctatctagtggtagggagaaacataaaaaaacagggagttcatattatgaattatatgaaatgaatatattgtattacatttgaacatttaattcaaactttacttgccctctggttataagcgttattaaaattattttcaaggttttgatttttttgcggtctctttaaacatctgatgaaagatgaaattctacttcccaaatggtcctaattgaaagcgaatgcacaaataagcatgctataagaaactggacagacatcatgagaaaacactcagtttcaccaaacctggtgtttttttttaaataatgtttcttcttttgttttctccttccttccttccttccttccttccttccttccttccttccttcctccctccctccctcccttctttccttcatttattaattggcttttaagtgctaactagcaagactgtaagccccatggagctggtaacctggtctttgttggctgtgctcatcaggtggcacaaaatagaagcttgattcatactttattggatgactattgaaagaactcagagggcaacttgccagattctgaatgtgtcctagagaataagtgtggttcctgtccacacagagtttacagtctgaaatggatttaatgttttccctaagatttaaacacatgctcaagaaaacttcacagtttgattaaatgagtaggcctaaaacagtgatattatcaacagagcatggggcaaaagtagactataagtcgaaaactttaaacacaatttcttttccattttcacacacacatacatacaggcatacatataaaagcatggctgattgttgctgttcaaaatgttcccattttagaaattattacaaaccaatatttaagtgctgctgtttaacattcagcaaaatttaaataaaagtggcattttaatttttttaattttatttttccataagttattggggtacaggtggtattcggttacatgagtaagttctttagtggagatttgtgagaacctggtacacccatcaacctagcagtataccttgcaccatatttgttgtcttttatcccgtgccccctcccacacttccccccaagtcaccaaagtccattgtatcattcttatgcctttgcatcctcatagcttagctcccacatgtcagtgagaacatatgatgttcgattttccattcctgagttacttcacttagaataatagtctctaatctcatccaggtcattgcaaatgctgttaatttatttcttttcatggctgagtagtatcccatcatcatatatatcagagtttctttatcacctcgttgattgatgggcatttgggttggttccacaattttgctactgtgaattgtgctgctgtaaacatgcatgtgcaagtatattttttgaatcatgacttcttttcctctgggtagatacccagtagtggcattgctacatcaaatggtagttctacttttagtcctttaaggaatctccacactgttttccatagtggctgtactagtttacattcccaccagcagtgcagaagtgttccctgatcactgcatccatgccaacatctgttttttgattctttgattatggtcattcttacaggggtaaggtggtatcactttgtggttttgagttgcatttccctgatcattattgatgttgagcattttctcatatgtttgttggccatttgtatatcttcttttgagaattttctatttgtgtccgtagcccataaaagtggcatttttaataccaaagtttaggaaaatcaatgatgctttatggctaaatctttaactgtatcaagacccattctttaagcctggcgcaaatcagtgctatggtggagatgataggtttaaaatgtctatgcttatctttgaggagaaaagtactgtatctcatgtaatttaatatatcatagtaaactatagaaggcagttgaagcctattatagtaaatttttgcatgtgtatttcaatataccaaactttcagtttgttgttacaaaataacatataaataggtttctggagctggatgggcacggtggctcacacctgtaatcccagcactttgggaggccaaggtgggcggatcatgaggtcaggagtttgcgaccagcctggccaacatggcgaaaccctgtctctactaaaaaatacaaaaattaggctggatgcagtggctcaaatctgtaatcccagcactttgggaggcagaggcgggcagatcacctgaggtcaggagttcgagaccagcctgggcaacgtggtgaaaccccatctctactaaaaatacaaaaaaattagccggctgtggtggcgtgcacctgtaatcccagctactcgggaggctgaggcaggagaatggattgaacccaggaggcgaaggttgcagtgagccaagatcgtgccactgtattcctgcctgggtgacaagagtgaaattctgtctcaaaaaaaaaaaaaaaaaaatgcctggaaaactcattacagaagtttccactgtagtaaaatttgttgaaataagttgcactcaatcatgtaaaaatgtggctccttgggactatctgaacggaatgttgtaagtgaaacaccctcaccatagtcactatcctgttatcaagaattctggatcttaaggaagtgcttgttttgtcaaaaatgtgacactaagacttttcacccctatagaaaaacctcaaccctggcctggcgtggtgtcccacccctgtgatcccagcactttgggaggccgaggccggtggattacttgaggtcaggagttcaagaccagcccgggcaacatggtgaaaccccgtctgcactaaaaacacaaaaattatctgggcttggtggcctgtggctgtaatcccagctattcgtgaggctgaggctagagaatcgcttgaacccaggaggcggaggttgcagtgagctgagatcgcgccattgcactccagcctgggcgacagagtgagactccgtctaaaacaacaacaagcaacaacaacaacaaacttcaattatgtttggaaagaagtgctaatttaatttggcaaagatgaagacagcagtcataaagcaaaacattcggtctcaggttgggtggattcccacctagttgacgaggccagctgcagattcaggtgggatcacctgatgatctttatcaatgccatttctttctctggatccttattactgacattagcaagggctttcagctgcccagaagatgttctttgcagacatttgctctcccgggctgccagcaggctttacaaatttaaaactttcagtgtaggaacccagcctccgtcgtccttcccctccaaagttaagagatctgctctaagggttcctgaggggtggtctggggccatgggaacaggatcaaggccccctgagcgccgggcctggcttctgtggcttcgcaaacttttcagcctgtgtgccacggcgacgcgcagcggctgagtcggagcccacgcggcgcgcgcctcccgcgaggaacttttcggcttgtaggctgcttgtcactctcgctttccgacgcgcctccccctggctcgcgctcccggagttccctcccctcctggcgaggacctttcccggcgcccgcggctccgatccccgccgcgctgcgcccgctctcccggccccggctgccccgctgagggctcccctctcccaggcaccgcagccgcgcccccgcgtcccgcctcccgagcggctcgcttcgcccgatgccccggccccgtcccgcgcactgagcgcctggcagcagggcgccgagtcccggggcgctgcggggcgctgcgccgagaacggccgggcctgagccctgggcggcccccagagccgatcagagcgcggggaggcgggggcgaggaggaggggacccgccgccgggggctggctgcttcgctccgagccgacttttcgccaatggtccaaagggacatgtccaagtcgcctcccacagcggcggcggcggtggcgcaggagatccagatggaactgctagagaacgtggctcccgcgggggcgctcggagccgccgcacaggtagcgagagcgcggcgccttctccttcctttgtgagccgccgggcagggcaccgacctcgggttctcccggcgcctccactgcagggatctctagcctcgcacctcctcccctcgtcgcctgcccaccctctgctcctctcctggcgccggggaccctgcccctttgcgccttttcctggctctgcctcggcttccatttttctctgcttccgaaaagccagtggggaagggcgggggagacctgccagtcctcccagacttctcccgggttgctccagctggccctcctcgccccttcccgggagaggcacatggagagacatgaatcaggggagtggactggacctgctgaagatcgtgagtccgggtgggcgggagggggcccgcttcccgcagcgctttctacgatgccgactctcctggccacgctccgagccggggtgggcgtgggtgtgaggatgatggggtgcaggtgggcaggaggggagcgaatatgggggtgccctgccggatccccccagagctgcccggaccacgctgcgcacctggggctgacagctctccagtcccctcgggcacttgccaaggtttgcctgtcccacctcatgcctttcccttaagaagcgagtgagctggggacaagaaagtttttatttttccgtctcccctgaaatgttagccatttcagggatctccaggatcccctttctctccgttgagtgtttgcggtttctggaaaaagtcagcttcgctgcaggttgttgtgaaattggagatgtcagttgtaggcgctgggcaatgacaaggtggtttttacg

SEQ ID NO: 8 - DNA, 6000 bp, Artificial sequence
  BNIP3
ggggtttctccatgttggccaggctggtctcaaactcccgacctcaggtgattggcccacctcggcctcccaaagtgctgggattacaggcgtgagccactgcgcccagcccgtttctttaaatatcatcagcaggccacattttctggtgtacgagccttcactggttaaccctgcagaaagtaacttctttaaatatcatcagcagggcacattttctggtgtacgaaccttcattggttaaccctgcagaaagtaacttctgtaaatatcatcagcagggcacatcttctggtgaacgaggcttcattggttaaccctgcagaaagtaacttctgtaaatatcatcagcagggcacatcttctggtgaacgaggcttcattggttaaccctgcagaaagtaacttctgtaaatatcatcagcagggcacatcttctggtgaatgagccttcattggttaaccctgcagaaagtaacttctgtaaatatcatcagcagggcacattttctggtgaacgagccttcgttggttaaccctgcagaaagtaacttctgcttctggctgcacccacctctccactgtttccaaacagatattctccaaattagctccttttgggattcctacatctgctttatctacccagagctgtaccaagaagaaatctcaacccccaaaactctggggactccctgtctgcaaacctagtccaataattctgaacacacttggcagcttttacagggactaggcacagaatcctcccaaatcctggaccactgagggcctctcccactccattcctgcctgtagaagctgagttctcaccaactgcctgctccctgctgtcctgccacacatgacagcttcattgtccactgttgctgccggaaaataaccgttttccttaaatatctgcatcagagacagcagaggcgatgacaatgggtgagtgaatccaaaatttaaggacagagaggtttatttcactgaaacaaagattccctaaaacgaagcaggtggattattcagtgcttgctggagagcattgaagagaggctccaaggccgggcacagtggctcatgcctgcaatcccaacactttgggaggttgatgtgggtggatcacttgaggtcaggagttaagagaccagcctggccaacaaggcaaaaccctgtgtctactaaaaatacaaaaattagccaggtgtggtggtgggcacctgtaatcccagctacttgggaggctgaggctgtagtgagccgagatcacaccaccgcactccagcctgggtgacagagtgagactccgtctctaaaaaaaaaaaaaaaaggctgcaggtgggagatggccttgggtgcggggaaaacaagtacataattcccagaggacagtgagattcacccaacaagccaaagtgtgagagctgatgggtagggctttggtgctccaccttcccggtcaattccaaagccccccttttttgaataaggactttagccaaggctcttcctgatgccttgccccagttctttcctaaaaatgtagattggaggagaactcaacaatgtactcaaaggtcagacaaatctctgcttagatgttttgaagggtttgtaaaaacctttaaataatattctggaatgcctgttagcctccaagatcattagaatgactcctcataaattctactttcctcagtggcttaagtgagggtttggttacctaagttaaaaagataccagcttaaccgggtgaatatacaaacccacaaattagtaagctatgcagatcaacttcctaagacaattcagaaggaagaaaaaaactaaggtctcaaagattatgaatttgcaactacaagaactgactaaccaaatgaggccttttaagaaaagacagggcctccttctaatgacaaaagggctgtttgcttctactgtaaaaagcctggccatttcaaaaaagattgtagaaaatttaacagcaggaccagtgaaaaaccaggatcccacatgatgaacaggattgctctgatgatcgaagggaggtgttcctatttctactaatgccttaggagaaatggacattgccataaatgaagaacagacacatgccctcaaagacactggtgccactcttttcgtccactttaagttgtctccttccttggagtaatggaactgtacaaacggtagggttatctcatcagcctatcactggatacaagtccaagcccttagaatcccagggccctcattcttttcttcattcccccacctctcagacatctcttaggcagacattttgtgttggaacgtcacaatgcatgctttccttctcccaaaaggaagaaatggatttaggtttagaatggaaggagcaaatggaaaaactacagaatgagaaattactgaaattataaaaatacagatcaaacaacttttttttcaactggcactaatgacacggatggcttagggcaagctttgtcagaccacttatggtcagagtcttccaccgacattagtaaaatatattcagccactttcattaaagtggaagtaaacctgattaatcctttacccaatatcagacaatatcctctaaggcctgaagcaaatgaaggaataagatccgtaacaaaagactatattaaaaggggtctaattattccttgcgccagcatgtaatactccaatccttcttgtaaggaagccgaatgggaagagctggtattctgtacaagatttgagaaccgtcatcaacactgtgatccccagacatctggtagtaccaaagccccatcctctttcggcagctggaagtgagacactgtgactcatttatgtagtgccttctttagtattccagtggacctagagtcagtatttgtttgcttttacttgggatgactgcgagcatatctggcctatcaggaaatattgctgatgaaactgctgcctccatcacagcccaacaaaaagctactgactcactggctaaggctgcactggacgactgcattgctttcgattattagctgagcaagcaagtctatgtatggtggcaaatacctcttgatgtgcataaaccctttccacgaagtagaaactcatatgggaaaaagtacaagccactaggctacactattctgaacaattctccatttgattttcttcgtaatacttttagttgactccctcgaataggttccgtttttcattctggcatacacattctctttttcattgtaatccttatgtgcaccgtacttggtaacatttttgttttaatagagatggagtctttctgtgttgccaggttggtcttgaactatcaggctcaagtgaccctcctgccttgggctccccaaatgctgggattacacacaagaaccactgcctggccagcactacagttctactatcgaaattattaatgctatgctatgtatctctggcggtttttttttttttttttttgagacagggtctcacactgttgcccaggctggagggtagtggcctgatcatggctcactgtagcccctacctcctgggctcaaggaatcctcccacagcaggtgccaccatacccagctaagttttttgtatttttttaggggagagaaagcgtttcgccatattgcccagactggtctccaactcctgggctccagcgatcctcctgcctcggcctcccaaagtgctgagatgaaagacatgcgccccacactggcctctggatgtttgttactcctgagaaaactaaaatcatggcactccaaagactagagacgattcaacaggcaagagcagggatgaaatgcacgcagcgactgaggtctggcttcgcggctctgtgaccccggttcagcttctgagcgcctggttccttggcgggacgcctcctcctttcccgcaagaccagacacgactgtctgggaagcagcgtttctggggcgcaccttgacacttggatttggatcaacaatgctttcaagaagaaagacttttgatcaaaagcgggaaatgagaaagcgactttcctctgaaaagtgcctcccagtcccgaggctgcgaggcccccacgccaggctggctcccacggaagccgggcacccacccggcccgaccaagcgccactccgccccgtggacggggcgtcccaccccggggacgcccgccccacaccgcgtttgcaccccggaggccccttgccgcagaggcggacggcgcgcctctcccgggcccctggggtccgcgcctccctcgggcagactctttcgactctgctcgagcctccgcttcttcctgcgggcggacgccccggacacaacgggccccgctgttcacgcaggggcgccccggcggggcgggcaaagacccggggacgcggtcccgtcccgagacgctcagctccggcccaccgctcgcagctcccgccccgggcgcaggtcccgaccccacgggccgtctcggagccgcagcggccgcttccctgcacgtcctcacgccccccgcacggacgccgccagccccgcgcctcagtttccccactagcaggatggaaagacgggccccgccccgaagcgtagcggcgtctccgtggtagccagtgcccagagagtccgccggtcccaccgccccttcaaaggagaacccggcccaccgcccgccgcggcggcgaccgcgcagcccactcgtcacgcggcccgcggcgtccagcccgggccggctcacctcaggcggtcgctgccgccctcgcgcctgcgcgcccctcgccccgcccctctccccgcccgcgtcccgcgcaccgcaggcctctgcccctcgcccaccgcaggacccgccccgcgcacgcgccgcacgtgccacacgcaccccacgcccctgcgcacgcgcaggccccaagtcgcggccaatgggcgacgcggccgcagatccgcccggccccgccctgccctgtgagttcctccggccgggctgcggggctccgctcagtccgggagcgcagctgggccgcggcgctccgacctccgctttcccaccgcccgcagctgaagcacatcccgcagcccggcgcggactccgatcgccgcagttgccctctggcgccatgtcgcagaacggagcgcccgggatgcaggaggagagcctgcagggtgaggcggaggaggcggcgcgggagccgagggggcgcgggggggaagcctggggaaggccaagagggcgccaaggggaggttgccggggaggcctagggggcatcgcgggccgggcgaggctgcgccatcctccccttccgtacccacccctcctgcgggcatgcggagcccggggcgtggggaccccgcgtactgcccggggttcgcggcctcgccactcgggcgggggttggcttggacccgggtcggaccgcacgggaaagccccgattctccagctccgcgcgagctagaattccacctggaggtgaatctgcgtctcgcagttggaccgaacagcctcaaagtccacgttgccctccgcggtctgtagttcagaccagtattggttttaatgaccaaacaccaaggcgtggcaagtggcctgttatgagctttaattttgttattaatgtttatatccatggtgactgttaggatttcctcaagggtgaacgcggagatgggagggggttacagcgtttttaaaatatggcattaaatgggcatgttccaatttcactagagggtcgttccaaaacaaagctttaaatgacttacgggttaagaaaacacaagcaaaaggacgctgcccgtgcagcactcagtcgttacagcctgcctaatgcccgagtagaggctcgctgtgtgcccttggctagattcgcaagaccatccgttcacgcagcgggaaacgcaggcccggggtgcaggacttgccccacgcacagccgggtggcgtggagacccaccccacccggtgggtccgcgtcagagtccagacgagcc

SEQ ID NO: 9 - DNA, 6000 bp, Artificial sequence
  CD248
ctgccgattgccagaggtggtctgacttcatgtggaaggccagtgagtgttggggacaagtgagctttggctgcaggaagaatcccagctccccactgcatctccctgagccaggttcatcacttctttgaagctcattcacgaggcatcgtgaatgagtcaggagctttctaggcttggggatagagcagggaaccaaataacaaccctgctctcatggagctcacagtctccaagggcagaagttctcccctgggggtgatgctgcttccagggaacatgacatttggcaatgtctggagacattttggatggtccaactggagggatgccactggcatctacataggagccaggaacactgctaaacaccttgcaatgcaccacgcagtccctacaacaagtaattaccgaggcctgaataccagtaactccaaggttgagaaacactggctaggggagatttgggccataagcaataagcaaatccacaagtaaatttctttttctttcttccttccttccttccttccttcctctctctctttcctttccttccttccttccttcctccctccctcccttcttcctttcccttcctttccctcccatccccacccctcccttcctctcctttccttcttccttccccttctttcttctcctttccttccttccttctctctttctctctttctttttaaaaaattttttttattattattatactttaagttttaagatacacatgcacaacgtgcaggtttgttacatatgtatacatttgccatgttggtgtgctgcacccattaacttgtcatttagcattaggtatatctcctaatgctatccctcccccatcccccaccctacaacagtcaccggtgtgtgatgttccccttcctgtgtccacgtgttctcattgttcaattctcacctatgagtgagaacatgcggtgtttggttttttttgtccttgcgatagtttgctgagaatgatggcttccagcttcatctatgtccctacaaaggacatgaactcatcattttttatggctgcgtagtattccatggtgtatatgtgccacattttcttaatacagtctatcattgttggacatttgggttggttccaagcctttgctattgtgaatagtgccgcaataaacatacgtgtgcatgtgtctttatagcagcatgatttataatcctttgggtatatatccagtaatgggatggctgggccaaatggtatttctagttctagatccctgaggaatcaccacactgacttccacaatggttgaactagtttacagtcccaccaacagtgtaaaagtgttcttacttctccacatcctctccagcacctgttgtttcttgactttttaatgatcgccattctaactggtgtgagatggtatctcattgtggttttgatttgcatttctctgatggccagtgatgatgagcattttttcatgtgttttttttggctgcataattgtcttcttttgagaactgtctgttcatatccttcgcccactttttgatagggttgtttgctttttcttgtaaatttgtttgagttcattgtagattctggatattagccctttgtcagatgagtaggttgcaaaaactttctcccattctgtaggttgcctgttcactttgatggtggtttcttttgctgtacagaagctctttagtttaattagatcccatttgtcaattttggctcttgttgccattgcttttggtgttttagacatcaagtccttgcccatgcctatgtcctgaatggtattgcctaggttttcttctagagtttttatggttttaggtctaacatgtaagtcttttctctttctttctttcgttcgttctctctttctgccgagaccagctcggtcggggagaccctaacccagcggtgctagaggaattaaagacacacacacagaaatatagaggtgtgaagtgagaaaccaggggtctcacagccttcagagctgagagccccgaacagagatttacccacgtatttattaacagcaagccagtcattagcattgtttctatagatattaaattaactaaaagtatcccttatgggaaacgaagggatgggctgaattaaaggaataggttgggctagttaactgcagcaggagcatgtccttaaggcacagatcactcatgctattgtttgtggcttaagaatgcctttaagcggttttccgccctgggcggggccaggtgttccttgctctcattctggtaaacccacagccttccagtgtgggcgttatggccatcatgaacatgtcacagtgctgcagagattttgtttatggccagttttggggccagtttatggccaaattttggggggcttgttcccaacatctttccttcttttctttttctctcccgctcgcctctcccctcccctcccctcctctcctctcctcttttcttttccacagtcttgctctgtcgcccaggctggagtgtgcagtggcgcaatcttggctcactgcaacctccacctcccaggttcaaatgattctcctgcctcagcctcccgagtagctgggattacaggtgcaccaccacgtccaactaatttcacaagtaaatatatttaatgtcagatagtgataagtgcagagcgagaaaatgcaggaagatccagtggtcaaggagccccagggggcggtgtggggtggggtgagatggtcaaggactctggtacttgagctgagccctggagaaggtaaagaagcagagcatttttatattggaggaagagcattccaggcagcaggaacagccaagaccaaggctgtgaggcagagtgtctggagcatttaaggaacagcaatgaggccagagtttggaggcagatgacatagaccaggaggccatggcaaggactgtggcccttcctctaagcgagatggggggctcagagggttctgaatggagaagtgatcagatctgacttggattttgaaaggatccctctggcaggatggagagggcaagagagacaccacgggagaggctgttggggaaatctagattggcgttgctagccacctgggccgggggtgggtggcaaagaaggtgtccaggagtggtcagattctggatctcttttgaaagtgaagccaacaggatttgctgagagactggatgtgggctgtgggagaaagagaggagtcaagcatgacctcaaggtttggggcctgagccaacagaaggatgaactcctccatcttgacttttgtctcctgagaaggggagtcattctgtgtgcttcacatgtatagaccttgtaagaaggaacttccaggcatatggcaagaacttcctaagccctggttctcgaggggctggctgggtctgcagggccagcctaggcactgtaaggtggtttgcaaaaacgcaccctggtctccacccaccacatatgctcagaagacaggaacatttgcttcaggctccacagctgacaaagcacatttgcaaacactgagcgctgtgacgcaatatctcagccctgctgagctaaaaatcctggactcattgccctcatgttgcaactgagaaaaaaggagacccagagagggccagtgactcccctgcagtcacaaagtcgatccatctgtggcagagggggaagctgcatcagggcagtttactgaagggcggaacctctcccccacctccccacactgtttctaacttctgctaagagtgcagcgggtgtgcatgggttaatccgccagccagctccccagaggccatcctggatgatgggctcagtgcacatgcctccagaggcctccaggaagggcgggaagaggaccctggccaggccgaaacagcaggccccgggggcagggagggctccacacacgtgatgcctgtgtcacatatacacatatatgtcactgtgtgccccatgcccatacatggccttgcatgggtcccctcacagccttccacatcctgcgtgcagcccagcccccacccagccccctaaaccacgcaccctgccttcctgacgcaggagcccagagaggcatttcctgtttaggggctgcctcctccccctctaagcccaggttcccagggccccaggctgagctggggtgaggggagggcagcccctggccccctcactcccccaacacccccacacgctggcccagctggaaccagaaagcttgagtatagggggagaggctgacgcaggggctcagtaaataaatgagaggctgaggatgcctgtgcctgggtgaccaagctgtttccattcaggccgaatcggaggtcttcatatgtcaggccatgtagaatgccacaccatttttgtatgtgcacctagggtctcagcatgtcagaatgtgtgtacgtgtggcaagggagtcatctgcaagccagcataggtccacggtgaggtgaagggacaaacagcttggcagagagtgcactcttgcatggggttgggggtgggggaggcgcatgcgcgcgtctgtggggcaagaaaggagtgggcatgagggtgttcccgtgcatggcgagcagctgggctgagactgctcccgggtgtgatggggctgctgtgtccagatttgggtctctgagtctctgggaagcgacctcaccccacagccccgagccccaacttgagggtcacagagctcggcaggcaggcttttcccaccccctgactctcagccccatggggcctggggcagccgtcaactgcgccttctcccctcctccgcccccaaccttagagccccccaccccactgcttcctgctctagcggcccccggggaagagggagcagggagctggcagccgccccagcccactccttacaaggcctgagcccggccccaggcccgcccccggcccgcccgcaggaggccccaggccctccccctgtcaagagctgccgccagcccggggccggaccagtccgggggcatcgcgatgctgctgcgcctgttgctggcctgggcggccgcagggcccacactgggccaggacccctgggctgctgagccccgtgccgcctgcggccccagcagctgctacgctctcttcccacggcgccgcaccttcctggaggcctggcgggcctgccgcgagctggggggcgacctggccactcctcggacccccgaggaggcccagcgtgtggacagcctggtgggtgcgggcccagccagccggctgctgtggatcgggctgcagcggcaggcccggcaatgccagctgcagcgcccactgcgcggcttcacgtggaccacaggggaccaggacacggctttcaccaactgggcccagccagcctctggaggcccctgcccggcccagcgctgtgtggccctggaggcaagtggcgagcaccgctggctggagggctcgtgcacgctggctgtcgacggctacctgtgccagtttggcttcgagggcgcctgcccggcgctgcaagatgaggcgggccaggccggcccagccgtgtataccacgcccttccacctggtctccacagagtttgagtggctgcccttcggctctgtggccgctgtgcagtgccaggctggcaggggagcctctctgctctgcgtgaagcagcctgagggaggtgtgggctggtcacgggctgggcccctgtgcctggggactggctgcagccctgacaacgggggctgcgaacacgaatgtgtggaggaggtggatggtcacgtgtcctgccgctgcactgagggcttccggctggcagcagacgggcgcagttgcgaggacccctgtgcccaggctccgtgcgagcagcagtgtgagcccggtgggccacaaggctacagctgccactgtcgcctgggtttccggccagcggaggatgatccgcaccgctgtgtggacacagatgagtgccagattgccggtgtgtgccagcagatgtgtgtcaa

SEQ ID NO: 10 - DNA, 6000 bp, Artificial sequence
  KCNA6
ttctcactggctctgagtggtttggattccacagtggaaagctcaagagtggggtgggtcattctagggagggtgttccgtgcttgtgcacaagtgttgtgttctctcagtaagaggtaagggagctgacgactcgccaccctgtcctcagagcaggggcctgtgggtgacaggaccatgaggagaaccattttcagaggttacacagagaaagaggaaaacttcagggatcgtattaccagaatgatgacaagtgcacacacacacacacacacacacagcaaaataaaaatgtaaagaagcctggaagacagatcatttatgtcatttatatacctggaataggtacattttagaacctagggcagaggtcagaatataaatggttatcacacaatgcttcacattatctgtgctcatcttgtgggcacatacgtccattcttaggtggttcttcccatggatgccccccacgtactcctgacctgttgcagatcatgtgttccactggaaggccagcgtgggcccaggaggatggggcccctggggtctagtcctggttgttggtttccaggagctctccatctcttggctttcctttgacttcacctacaacccctcagcctcctatgctggctgcccttcctcctgaccttgaccggtagattgaagactgctgtgaaggacttggtcctggtcatttctcctcgttgtccatattctgtttctgcagaagaccttggtgccatgaatgtatatatttctagatgtcttaaattcataaatcgtaagtttctttttcatattccagacctaaataaataactgcctccttgatgtcttaatttgcatgtataatttccctctccaatgtaaaacatctaaaggtgaggtcttgacgcatcccaacttgtttaaccccttgtcttccctttctcagtaaagggcgacacctacctgcctagttgcttgagcttggcattggctgcttatttctcatgttttattgatgactaattcctgctgattctatcatctaaacatacctcctcagttctcctcacctcctctccactctatctctactgactctacctttctctggcccaagtcaccatcatcttttccccagacgactgcaatttcctggtctttctatttcttcttctacctcttcataattcattcccctcccagcctctaagtaaatcacatcacgtctctcctctgcttgaaaaccatcattggcttcataccgcccttaagataaaacctgaacacctcaccctataagatggtacccttgctaattcttcaatcttatcttgcacctccctttcccttgcccattctcctcccagcgcacggccttctttttgaccttgaacacccatgacctttctagctcagggcttttgcacttgtcagtctggaatgctcttccctgcatttggcactgtggccccattctcttatctatttttgagacagaatctccctctgtcacccaggctggagtgcagtggtacaatctcggctcactgcaacctccacctcccaggttaaagcgatcctcctgcctcaacctcccaagtagctgggactacacgtgccctccaccatgcccagataatttttgcatttttagtagagacagagttttgccatgttggtcagactggtcttgaactcctaacctcaggtgatccacctgcctcagcctcccaaagtgctgggattacaggcatgagccaccgtgccaggctccattctcctctttggattgtcagttgaaatactacctcttcagagggaacttttccagctacccttgcaaagttgagtccccaagttcatttctgacatggcatcccatttatcctcctcaaggcatttactgcaatatggaattattttccttattgtctactgttaaaattttctgtctataatgtaatttattacatgaaaatgtatgcttatcaaagcagatattttgtatagatcactgctaaatcctcagcacctggcaccatgctggcactcagtagctgctcaatatatactttttaaataaatgaatgattgctatgaccatggcaaacatctgaatctggatacaacaacaaaaaataacaacaaaatctgtgctggcatgaaattggtgcttggtgaatgctggggaaaaatcccctgccattggaagccacttggcagtgtttatggatggagctgtgtcttccttcacctgggaactggcatttctgaaggtgtgattgtactcatccttaacatacgcttggggggaatgatctaggcatagggtttttcttaagctaagaaatgagtctatttcacaaaagaactaaaaacagaactctgattcatgaatttgccaatggcttgtccttctcaggggaacctccctgggcacagtgaaacccttcttggggcacgacctcatgcttgatgtgaaggtcatcacagaacatcagctctgcctgaagggtggccggcagcccagtcaggcttaacccgaggggtactcaaacggaggttcttgacaccttatcagggtgtgtacgcttctgcagaacgaaggttccttcccatgctttgccaggattggttttgaagaattactgacctagttgacagtctcgtgaataaaaatggcaatgatgaaaacaaacctgcaaatgaatcattcattatgaatttgaaaacaaaaacaaaaagaatcaggggttagtcaatttatgactcagaaaataatgacagataactggataagggtaaagtgacagataactggatgggggtaaagagtatcttctatagtgtatttgctgttggtttccttttttttcttttctttcaaaaagattttttaaacagagcaacttttgtgtggagtctaaatggcttaattacatagtctgtaagtgagaaaactccgctggatgactccatgtacttgctttctctcccttcacggaaacatgcaaggtgagagaaaggagtggaaatgagacaggcaagcatgagatgggcaagctggctgctttcctgttgtgatgtggtttggagaaaaaagaacaaaagccctttactcaagctgtaactcccagccagtcagcatcaaaggcccaagaagctattaaccacaaattaattaaccccttgctttagagaactaaggacctttctgaggccctacgtgcctagctaggcttaactttcacctcatcatgaacttttccttattttagtactaaaaatcatgcccacaggtggagatttaagatgctaatgagacatatgttgtatgaagcagcatcttaagccaccgtacatgtgcccgaaaaacctcacctctacatgccctgacttccccttactgcagacctccacaaagggaacccacaccttgactttggagagcaacccacttcctttcttggtgtttggtcccttatgtccataagctttcataaaatctttctctttgctactgtatgctgtgatctcttttgatttctatcctgggggatcaaaaaagcccacagggcgttggtgccagaaatcagctgtgcaagcacagaaagggagcagaggctgttgcctggctcaactggtgaggagcaagactggagaatgagcatgcagtgagttctaatactagcacatttcttacccactcaacacctgatcccttcccgacacatccaggagccttggttctctccttcctccaactctctgagagtgaatgctgctgagaaccactgtggttttaaaatactccgtgcaatacaaagaactctcagatgcatcctgtggtttgagagcacctttatgtgtcattctcatcattaacctcaatgaatgcatactaatgatctaatatgcaccagaagctacacaaaaacactttgtagttgttagccaattcattttggctacagccagccagaaaagaggaaactgaggcacagagaagcaaatgtcaaccctacctggaagtgtcagtctgaaaagatcagttgagtcttcctctaccagaccccaacttgtctggtctcgtgaagaaagacagaaatggatggaaaaccattcttatcaccttcttgctggagagttagggaagagtcagctggtccctaacaacaaactttgccctactccagtactgccaaaggggtctttgttgtaacatggcctacaaggagaggacatctttctgggaaagcacctagaatgtggctggcacatattcaatgcccagtaaatgtagctattattttttcaatcaacactcttacataccccttttccactcaccctcttctccctttttttggtcacccaataagcagaaattccagatgcatccttcggggtggttgggagcaaaggatccccagggtgcactgtgccctgggctgaggtatctctgaggtgcttccatcttctgggtccttcctcactggggttgttgcctcaggctctcaggactcccctggaaatcttctgatgggaaaggcattgattgacttgccaccctttctctaccatccctttgcaattctgggagagttgcagccccgccctcccaggcttgcaaaggtagacggagaattatattggaatttaaatcggaagctctcaaggcatctcaaaaatactttctctattttttttttcctgtagatattggagaggttggcaaacgggtcttcctgaagacagaagaatgtatgatttaatgttttctttagatttctgtatgagtggatgcacagtgctccgtattgtgtggtggggcggggtgtgtcttcttattgatgaaatacactgcgcaggtcaactcggtaaattgaaatgagaagagccgactgcgggggtggagggggtgtggtattagggtgccggcgcttgtggaggggggcgcgaatgtgaacgtgtgaaagcgagaggcgtgccaggagagcgcgggaaagcttactggtgaggcaagtgtgcgtctatttccatggcgccctggctcgcggcagcccctggctgggcgaggggtgtgatgtgggagtggggtgggagggggcagcaggcggggcctgccacgtcacttggagagtgtgtgttgggaaggaagggcagagcggagagccgagccgctgcagctgcggcggcggcagcgaagccttgagccgtggggaggtgggtccccgcgctcgggcgccggggcagccccgggccctctgcgaggcctgcggcgcggctcctagggaggaggtggcggctgtggcggccggaaccgcgaccttggccggacccagccccgcggtggacgcagggcggaggccgagccccgccaggagtctttgccgagccggagggaggcgcatctggcgcttcggtaccagcggcagccgggggtccggagcggctggaggagcgcagtgggaactgggaagagctagcccggctggagggcggacctctgcgtccgggagccgggtctcaggcaccgctgggggcgaagccacgcgtcttttcgggcagccaatttcacacgcgcctgtgtgcggttccgggcatcccagtaagctctagcacccgggcgcgggtaacgggaagcgcagaaccaaatccccagcgcccaggtcacctccccagacccagccttgcagggaccagggctttagggctcacggacccaacggccaggtcagaccgcgaaccgggaggagcgcgggccccaccctaaagagggcgcagccgggagctggggagcgggtgccgcgctccagagattgtgtcgtgggcgccgtcctagtggcggggagcgcacctccgagggggcatgagatcggagaaatcccttacgctggcggcgccgggggaggtccgtgggccggagggagagcaacaggatgcgggagacttcccggaggccggcgggggcgggggctgctgtagtagcgagcggctggtgat

SEQ ID NO: 11 - DNA, 6000 bp, Artificial sequence
  HS3ST2
tgattaggcccaaagactgaaggggaagaaaatttcctattttatgattttaaattcaaagtcttgaatgcagggcattagagtgggaaggaagattcaagagaatgctaatgtgaaagagaaatggactcagaaaaagaaaagaaagagagtgtaatgtgctctttggttgctattctatttgagcccagttctccagccttcttcttaattttgtgagctaccccaaagttcctttcctgtgtaagatatcccaaatctctttttttcacttctagtaacccttactgtttcataaataaagagcaatggcagttaagaatgagaagcaaaataaataagatacaagaaaagtagagaagttgaagaaagtaactggatcctatgactgtctcaaatttggaagggttgcagtcctgatacatgcatatccttcaaaataagcccccccacctccaacactttttattaaaatggtatctattccttgcaacggagggagcaactctttttgtttgtttgttgtattggtcagcactctttcagttgcaagtgctaacagaaagatagctaatcactgcagcaataaagtccagggtccatgcattccgcatggctagattcagaggttcaaaggatattatcaggaatctaattctccctccatcagttctgctctcctctttgttggctttattgttggtttctcctcttgatagcagagatgaccattggaagcttcaggcttattttagattaattcatcatctcagaaaaagaagtttctcttttccaatagttttcacaaatgttgtgtggtttattctcactagaaagatttgagttactttcagttcttgaaatataattagaaagtgatgatgtcaggcttggtcatatgcctgtctttgaactatgttcaacccatacaaaccacatgggaataaaagagggaaaactgtttgtccaaggatggcaaggattactagttgtcctccataaccattctttagttctttcttgataccaaaatctcaaagtgttagccaggcatacagccatccagaatcaagactgcattttgaagcatctcttgaagctagaaagtcaaatgataaaatgctgtcaaattgaatacgagttgaaataatacaggcaacttccaagaaatgtccttaaacagatatggcatgtctttacatatttactaatgaccagaatgtgaacattctggctgcagatgaataagtcattgtagactatgtggtgactttagaatggaggaaatgcatggtagagtaacaagatagagggagcctgggtcccaaacactttggagcttccatacaagccctaaagtaccttcatctggactttcatgtgattacaaaaataaacatataccttgcttaagtgggtgttaattgacaaactacacaacccagcataatcctacctgatacccccaaagcagaatcacgtttgctactgtaaacagataaaggtttaggagatgggcaggtatgtccaatgtctcacttcctaaaaaattctatccaagtcagtcattactctgattggaaatttgaagaggaaaatcttcctggttttgatatatgtgagtacccagggatatcacagtttatgaattcaaaggttaaaaattatttaaaaattaagccaagcacagaattatgagaaatcattatattcatgttttattttctaagggaaatagtactgatcacagcagatgactccacatttaaccttgtaattttaacaatggaatacaaaaatagcaggttcatgatgtgaataattgttcaaagtatatataagagctccttccaggccaggcattgtggctcatgtctgtaattctagcactttgggaagctgaggcaggaggatcacttgagctcaggagttcaagaccagcttgggcaacacagtaagacttcatctctacaaaaaattaaaaaactagctaggtgtggtggcacatgcctgtagtcctagctactcaggaggctgaggcaggaggatcccttgagcccaggaagtggaggctgcagcatgagctatgattgcactactgcatgccagcatggtcaacagagtgagacacttcccctcacccaaaagaagaagaagaactcctttaagtatatttgttttgacatttttattaacaattagaatgaggaattaattaaattgtgatcaactgtggaaattacatgatgccaagtgagtggttcatgctttgggtaatggaatcatagaatcatgcaacatctaaactttgagagacttttaaggtgaatcatagcgtcccattttacagctgaggaacctgaggcttaaagggggtccacttgcccaaagtacacctggaataaagggtaaagctgggatacaatctttctactttttctttttgaataaatgaaagctacctcgtggtttgacttcaaatagacatttaaaaaaaactagggcagcgaactgatgagacaagatgaaatgcaatcaagtcaaatctataaggacctacacattgggtccaaaaaggcccactgtacaagcacagtatggggtagctgtgccttaatcagcagcacttgtaagaaacatttagagatgttacttggctgcaagtttgagatgtcagcagtgtgatatagccacaagaaaaagataatggagtcttaggtggcattactagaaatagaacttacaaaagagaggtgagagtcctgttgaacttttcaatccatatctgaaattcctcgcgcagtcccaggtgtgagattttagaagagacatagacaaattagcttacatccaggagaggagcctaggatggaattatatgaataatggtcaaaggaagttaggaagctgcaaagtgctgtaactaagagcttgaatcttggagtcaagactgcccgggtttaatcccagctctgccagttactgtgtatatgtttgttaaatcttcttatcactgcctgtagattagaaattacaataatacctatctccaaagataaatgaggtagtgcatgtcaagtgattagcgcataactcccataaaacaagcactcaataaatgctagctactattagaattaagacagcaaagtgatgccagggtaatggtaacaacttcataggttgtgaatatttaatcagttaacccatgccaggtgcttgatacaaagttggcagctattattattatctgccgtagaattggtttaaggtttctagggatgggactagtttggggacaaaatattttctggtttgggctaagatccacagaacctaatgatcagtttacagcctgaggaaggaagtcagttataccctgatcagggtgggggtcatggtggtcatctagacattctatggctgggtggtggtggagggcactcaccttgtgaacactcggacatggtgaattggcattggcattgctgttgaaggacaactcagccgtgttcttagccatggccatttaggcctgttctgatgcagggttctgatccaaggtaccagtgtggtccctcagggaagtactggggatcgtcacttatgcctgttctggacatggtcaccgagaactgtcctgtaggcattcacttaggaatcattcgaagtggaattgctcctggatacgttctccttgtactctgtttcctcctcctagtgtctctgtgtgaagaagccctcctcactcagccctcggcgaccctctggtaccctggacagctccccggggagcagtctaccgctaggcggcggctgctaagagaggaaccctcctgacgcggagtctgccgctccggggctcgctctccggcaggcccggggagaggtggggtgacaatgggttggggtgcgcgcgtgcctcataggtgcgagacagagcgagccgccggggtgtgagtcagcgcgctgggggctaagaagctgggtgaatagtcacggaatctcactcacgctcggctcctccacccatcccgtctacagcgcgtgtcccagtccagggcgtgcgtgcgctcggtgtccgattccgggctgtgtgtgtccatttggcgagatgtcgagagcggggggagtgtccttgtcggtgtatctgggcccaggttaggggacttctcctccccacccccgcgtgggtgtgggggtgtgtccgggctagggcgcgtgtgcttctgtgcctgtgcgtgcgtgtgcgggtcagggtggtgggaccgcgcatcagggcagggtgcctgcgtctgcgtctgggtctgtctggtctgcatgtcggcgcgatctcgacctggattcgtgtccctggatgtcgagaggccagcgtggtgggggtgtccagcctcccggaggagtactatgccttgacaccttcgtttcaccgccccaaagctggcctggggctccgtagggagtggcctgcatggggagggcccgcgtgctgtgtttctgggaggggtaagagagtgggggcgcagggggcgggccaggtccctgggcgcggcgcgggctcgggggacccgcgcggctgacgtcaggccactccttaaatagagccggcagcgcgctccgctcggcatttcccgaagagccagatcgcggccggcgccagcgccaccgtccggtccacccgccagcccgcacagccgcgccgccgccgagcgtttcgtgagcggcgctccgaggatcaggaatggggcttcgggcgctgggcgcgctccgaacccggcgcacgtaagagcctgggagcgcccgagccgcccggctgcccggagccccatcgcctaggaccgggagatgctggaaatgcaaccgcctgttccccgaggagccgctgcccccgggaccccctggcactgtgcgcaccctggtcagcagcccccggagaagacggcgcccccaacgcccgacccgcgtggccgtggcagcgccacgcgagccctctaggcgaccgcagggccacagcagctcagccgccggtgccccctcggaaaccatgacccccggcgcgggcccatggagccatggcctatagggtcctgggccgcgcggggccacctcagccgcggagggcgcgcaggctgctcttcgccttcacgctctcgctctcctgcacttacctgtgttacagcttcctgtgctgctgcgacgacctgggtcggagccgcctcctcggcgcgcctcgctgcctccgcggccccagcgcgggcggccagaaacttctccagaagtcccgcccctgtgatccctccgggccgacgcccagcgagcccagcgctcccagcgcgcccgccgccgccgtgcccgcccctcgcctctccggttccaaccactccggctcacccaagctgggtaccaagcggttgccccaagccctcattgtgggcgtgaagaaggggggcacccgggccgtgctggagtttatccgagtacacccggacgtgcgggccttgggcacggaaccccacttctttgacaggaactacggccgcgggctggattggtacaggtaaggaccaggagctccgctccgtgcgccgggtctctgatcgcttccattgggagagccatccgtctcttgtgttttctctttcttttaacccaactcattgtatgggttcaggctgacacacagggccatggggggctatagcagaatttacccagaacttcccagtgataatctagacgggcagtttctggaactgcaaagggcgttccctcgtcactggagtcgttggaaaaggattatctccagtcaaacctaagtgccagctaaagggctaactccctctgtgaccagcccttagggtgcccaaggaagggacaggcgaggacctgtgctgcctgaacacggcaccatcctaaccctctgtaggtctttgctggtacccagcccctgaaggaccctgagaaagataaggcagttcagagaccccttgcagcaaggct

SEQ ID NO: 12 - DNA, 6000 bp, Artificial sequence
  CEACAM4
aaaaaagtacaaaaattagccaggcatggtggtacatgcctgtagtcccagctacttggggggcgggggctgaggcaggaggatcacttgagcctgggagattgaggctgcagtgagccaagatcatgccactgcactccagcctgggaacaagatgagaccctgtctcaaaaaaaaaaaaaaattaagatcacattcaatgtgaaaaccacacataggctatatgttgtttatgccatataatttaaaaacactacggaggagtttccgcatcagtggctaaaacaattccctgttggttacatatctgcctcatattccattgtgtaaatgtatataacctgtttaactatgctaatgtaaatgtaggttgttttcagtattttgctgttaagccatcactgccatgagaaaccgtgattaggtgtcatttttcatgagtgtgtgataaattccttgcagtgcagtttgggaggcaaagagaatgcatatgttaaattttgataagtgttgacaaatttgatgccacacagttggtacaaattttcatgtcacccaacaaagttaggaagtacaatttttaccaaagttcttcccacacgttgtgttatcaaatggaatttttcagcatttaaaaccactgggccatattatgatcctcacctttccaggataatatcaatgtccgtttctcctgcatgtgtgcgggtattttgttcatatgtctacgaatcattcaaattgttttttcttctgtaaactgtttatatatttgcctacttttctactagacttcagtctttttaattttatttattctttaagtattaacagatgtgaaagttgtcagagtcaaaatgagtcactagtgtgaaaaaaaactctgacaaatagagccagagaagaccatgaagagaggatcctcatgcctgataacaaaactatcacaaaagactctgcaaaagccacaagtttatacaaaggccatcacaaccttatatgaaaactacttctgcaaggacatctgcccagcaactgcctatagaacctcacagtggcatcattctggctattgatctttgtagctagttttttttttttttttcaaaatgactagataataatcccaattttttcctttaaaaactcgaatatgtagatcattttactatggcacatgcatttccattgaaatgtgctactcccaaataaacatcagtttctcatagaaagcctcactctgtttgttaccatatatggtgtcagaagtgggatctgggaaagatcactatcagaagaaatctgtgatctttgaaccagtgtgcactactcacttgagaagtttgagctctctgcttccacactcacctttcctgccctgacaagtctttgctcaagcagagcctctttttggtagaagctcttgactttatttggaatctgatttggataaggctgccttagtaaaagaccatacatttctcctgggatgataaaaaaaaaaaaaactttttgtcttttctagcaagtcctttctgagagaaaggcgtatatctttctagatcacgtactctggattctacaaaatttacattctgcctgtgaggcaagtctattctggtgaatttacttccattttggcctgtgtgcctaatttaaatactttaaaaatctgcatgcctgggttaaaattcttgtgaatgctcttatctggatttctttttatttggtttgactcttttccccttgcttgcttctgaaaatcatcccagaacacaaaaaaatagacattctaaatgacgggcacaaaatggctgattaacagccactagcgtggttgccaccatctaaaacactggtacaaatgcctgacattctctggcaggatttgtaaaattttcttcactttcaagagattaataagaaatggaatggggctctcaagcattaaggcatgccaggttttctggggctccagctggctacattttatggttctttcttgtgcacattttaaagtttatgagcaaaattacatcaaggaaaattcagtactcaatgatcatcattcaacctgttttaaaaagccctgcatctatagggtggaaatgtagagtcttctaaattctctatttttttttctctacctactttgaatctgctgacttttctactggtgttgagataaaactcactgcttatggcattctagcccagagttttaaaaaagaaatcttgaagggctttaaaattaatggctttacaaattaaacaactccatgataagaaacaacttagacagctttaggaaatgtaaatttaagtttgtctaactaataattgcttataatggagcacaattaaaaatcaataattaaaaaaatacatggttataaaagttaggctctcagatcatacaggtcaaaatcttgaactcagagcaataatttaaggtgtctctgtccgacataaacttttttcttcttttgccatgcagaggcaaaaaagaaaaagccaggaaaaaaagctaaaatccttcctcatccacatttgttaatcaagcaaaccacatcaccaccacccgcccccctccaacccacacaaaaaatctagtttaaggctagttggagatttttttttcttatacaattcagccagttctagctaaagtgtaagcaattgaaaatttaatcctaaactcatgtgaaacagaaaaaaaaaagatgctgaaactgtagaggtttcatttgtttatttgtttataagttacactgccattagaaactgctttacccaaaatatttcccccagccttcattatattacctataggggcaaataaagtttatccatgttaacgattccaatttgtcagaaatacaattggatccagttgactttaatcaactagtgagtttgtattactatctcatcactaaaattctaaaatgaaagctgtaagatttttatttgtttgtggatatgtgtttaggtgtgtttttgcatatgtacatgtattatggtctatattgtgtctacatgacaaaatccaccttagttggccagaaacgccttaataaactctatttgcattaccttagagaaatgagcataagaacatggcttttaccctagtagcatcgggagggaattcagggtctttctaacagtgaggggcaaacccaattcattccctgacatcattcactgacattccctccaggctctaacatatgtctttctcacaaacacaccaaaactgacacaaactccggatattcgatcccaggtggatcttccaccagggcagaatgaccacaagaaagtcagggagtgattccccagcctcgagatccccagtatttgggacatctgcctatggtccctgcagacatttcaccaggggatccaggggaacttctcctgcaggaggggacaggataacccaggatctgcctttgtttccatctcagagggactgagggtcacggggcctcccctgctctaatacaggaaccaggtatcccttgcagcctgcaggtaggagctgccccagctcctgggccctgtggagaggcctggggcaggtgacagacagggacacagatgacctggaggcggaactcccagtgttgtgatggaggaacacagaacacaccgaggaccacctcccaggccagtgccctctctctaaacccccagagacacctccctgggcccttcttttgaaaccttggggacggatggctctttctgaggcagcccatccgcctgcaggacagttctcccaaatcaggaccaggagtgctctggacaactctcgtcctctccctgagctcatcctgcactgcatggagttggacatcctggggacccacagtgaacaggaccaaggatgacctgaccctgcagtctggaggtcagagcccacctctgcccaggggccagggccaactcataccacgtggaccctggtcagcatccctggggaagcccctgacttttaccacagggttcctcttgctctccaggggcaacattgcacgcagacaacacaggaaatggattcccctggacaggaatctggctttgctaaggaggtggaggtgaagcctggtttccatactttgctccagcaggcccttccagtccctcccatgtgcctgctctgtctctcctgatccttcctggagcctctgaggatcctgctctgccaggattctctgctcagttctccactttctcctggtatcatgcatggggaaggtacagtgacaacaggacaatcaccttcacagaggacagaggccacccgggatggtaagggagaacatgcacaggccctaagccacagctcagccaacagaaacggagagggaggatctccctgaatccctcctcaaggacagcagaacccagagccacccacctccctccaccacagtcctctcttcccaggacatgcaggacacctccctccacatccaggagctggggatcctcctgagacccccaggcctggatctctgtccctgggtcagaggcaaggctggtgacactggagagagaggactggtcccccccgtagtcgccccccattttctatcccacagagccacctctgtcaccttcctgctgggtatcatctcacactccctgagtattggggagcatgaggagacctgggggcccagctgggtctctgtgtcacaaaaggaaacagttccccaagtttgggagaccccagagtacctctgtttgtggtgacattcccaaagggtcagtgcagaggtgacaagtcaccctctctggggacaggggactccaccaaccctgcttctcaaagtgtggttaggaaactgtaatgtacacagaagagaaaggggaaggagggacaaaaaaggcagaaatgagaggggaggggcagaggggtgacctgggaagagccccgcctctgcccctggccctgggaagtgcttctgcccgggaggaggctcagcacagaaggaggaaggtcagcagccccgacagccgacagtcacagcagctctgacaagagcgttcctggagcccagctcctctccacagaggacaagcaggcagcagagaccatgggccccccctcagccgctccccgtggagggcacaggccctggcaggggctcctgatcacaggtgaggggaggactctctgggagtggtgggaagagggagcacagagactgactggggtctcttgggtaggaggggatagagggcttctggctggggtctcctggggctctgagaggggactgagggcctctgttggaggctggataagggagagaacatcagagaggggcaggggtcacaacaggaaaatctcagtgaactggaattggtaaaaggcaggaaaatctcaagtgttctctcgtcctggttaatcatcactggccactacattttgaaaaatgataataactataccagatgacacttcaaataaaaacataaccagggcataaaacactgctcttagccaacaacctcagacactgggaaataaacctcaggacttggaggccctgagaatgctcatgaactcatctacaggagtctgcagcctgtgccaggcactggggtgcaaccaagatcacacaagtccccgccctcacagagctcacgctctcatggggaggaagacaaacacctaaagagatctagaatgtgaggtcaggtgctgacaagagccctggagggaacagagctgggaaaggtcagaaagggaagacccagggtctctagaggaggtgtcaggggaggggtctcccaaaaacaccctgatgtgagcaggatctgagggcagtggggagggagccgtgcagacccctggggaagaagattccaccagggaaatgccaaggtccaagctgttgaaggaatgggggtcatgctgctgacccagggacacacacacacacacacacacacacacacacacacacacacacacacaca

SEQ ID NO: 13 - DNA, 6000 bp, Artificial sequence
  NEFH
tattattgagatggagtcttgctatgttgcccagcctggtctcaagctcctgggctcaagtgatcctcccaccttggccttccaaagtgctgggattacaggcataagctaccatgaccagcctctcttcatttctcaaaccatgacatggggatagtaatggtatctatttcataggattgcaagaataagagaggccaggcatctcctcctttccttagatgacagccttcattcccattgctacttagcctcttgctctaggtaagaagagtaatgacagccctggggcacttgtaacttggcaagccccagttactagatgaaagtccaataaaccatctcactctccatcttgggaaccacagcccaagaatcattcatccataatctcccacagcaggaactagagcaggggagatagggtggacctgtgaccccatccaccagttcctggcctttggggtagaatccagcttcccacctggtgcagtcccagggcggcagatgggcgggcaggcaggcaggcagacctgctgcaggcctgagtctgcggtggctggtccctgggggttaaggctgtgggtggaagctaggagggggaggcttatgcaaaactgcacacagtttagttttttgctgtctgtgtcctaaacggagtagacatggggagactgccgcccttttattataaatcagattcaaacgcggcaaaaagacggcatctctaaggagggagaggagaaaaaaggagggagaggaaggaaaaggggaggaataaagaggacagagaaattggggggaaagagggagggatataatagagggagaaggaagggaatgcagtggagagaggggcaagaggagagacggagaatgtgggacatgacggacattcacggggaaaggaagcaagaagaaaggctcagcacttcctaaaaggaattgctgaacataaaaaggaacaggctgtgcaggagcagggtgagtctgcagacaacaaagccagcaaggttgagcctcagcaaaggggactacacagctcgggctgggaggcttcaggccccaggatgggggccacactttcaagacctccttctccagtcaacctgcgggcagcctccctagggcctggaactgtccagatgcccagtgctaaaatgtctgggtgatcacgggcctctcaggcagagcctcgcagtcatcctgaaactatttgctacgcacccactatatgccagacaatattccgagcactggagatgtcagcagtgaataaaacaaacaaaaatccctgtcctgatgaagttctatttagtggagaagatgcatagtagcatgtcagaaaacgtcaagtcctatggagaaaaaacaagcaggaaaagagaacgaggaatttagcatctgcacctcaaacagtggtcacatgtagctcccacagaggtgacacagtgtcagagggtacccaggccgggaagtaggttctcacatacaggctatgctgatgttgggtcatgggccacagagactcacagatgggtgatctgtcagaggccagaccccaaccccagccccagaaagctgcccatcccctctctgaaggcctctcaatcctctaccctcagaccatctaggcctctactatgcaagagaaacaagtttccaccctgtgtcctgtgccagctcaggtctggatgagttgagacactgggctctgctgtgtcactgagtcaactcaggcaacccacttcctcactcgggtctcagtctacttgcctataaaatagggacaatgatccctgccgagtgaacagtgagtagcagcgaggctggtggctgtttaagcgcctactgctttatccttcaccctgcaagtatgttcctgaagagcaccagccttggggtccacaaaagcccagggacagcaggggctgagacccaaatctcagtgtgcctttcagtgtgagccactggacccttgtcctggagacacttggggacaagaagggcccaagagcagttaagtttgggaaactcagcacactctacttgcctctgggagattcacaatgctcactgccaagtccaggctttgggaagataactccaagaactgcgggtggatggatggatggatggatagatagatgaatatatcgttctactcacttccaaatggtaagaatcacagggaggaaagtgaaagcgattcgagccccaaggactcagggcagaaccattcacagcccctccagaccacaagtttggctggaaaacagacggaacttattcattcattgattcatttacacaccacagagtggtaagagcagcattagagattagcactgggaaccatgggaaccaccaggaggggacaattaactaaatgtgggggtgaaggcggccagggatggctttctggaggtgagcctgaagggtcctttgggggaactgacctcagggctccagccctcatgccattttctccagccacaagagtcatgcttggggcttcttggactacataggcagcttcaatctgatggctgtggccccttggcctcaacagaatacatcttggagccccctttttaccccaaacccccattcctccttgctgtcagctgcttgtgagccttctcacatccagagaatgtatcagcattgtgcagactgaaaagacccagaggaacaaggctccaatggcaaaattccaagtagaatgacaaataaatggggagccatctgagagcaagggagtcctgcccaacacccgccccatgcctttctcagggacctcagaccagccactcacctccatcctcccagcaccacctgcaaccagccccttgccctctgcaaactggagcacgactggatctttagatgggggaaaaatgcttcatcatgttctgctgcttcatgcaaaaccagaaactccctccccctcttccctcctcccagcgcactctccttccagtaaaaagtggttaaagggacagcgccatcaatttcccagctctgagggtctgcttagaactagggggctggaaggagacagagggcaaagagaaaggaactggcagaggtctttcctgggggatatgtctgttctgtcctggggatcctggagcaggaaaacccgcgtaaagtaggggtgtagtgggtgttgagataactgcctgggggaggttcagagtggaagtacgagtctacaaactctcaagggcgtctcagggctcccagcatccccaggggtcctttcgcaggggtccctaagcaggaggggaacagcccagaaaacacggaactggacccccgacaggaagtccagggaggggtccctggctcactatgtgaccctgctggatcacttgcctcccctctcgggtcccctcagcacagtgtccctcccttccttcccctaaagtaaaagcagagggttaatctctttccccgccccacgcccaacaaagagcaggccctgtccccggtgctgaagcgccagccgcagcaccacccccactcccacagcataaaacatgagccaaaaccaataaagagccaaatgtcacagccgttgcagggccccctaaatcctggggaccccttcttctacctgacatcctattggggtgagggactttggtactcagaaagcatctcatcacttccctgtaagagagaagggatgccgactcaggcgcctgcttgtctgttacaggagtgggggaagagaggacaagttgaggctgagaagatggggagggggagggagaaaagaggacttcctagtgttgacagaacggcaagatgtgggttccccatccccagttcagccagagacccctcaaagtggaacttcctggggcagtcgggggtcaggagttggagcttgtctctggggcaagaccccttcgttgtacagatggaaaaacaagggtgggaggacacagcttgtccaaggtcattcgaccagcaaactgcctagctgaccccagtgtgcagaagctggctcgggtgacacccatcatttccccccaccccacacaggggccagctctctcaacttcatgcccaagccctcctacggtacccccactgtaggttctctgcccctcaaactcagcccagctttctcctgcctgttcaggggaccttctgcccgcttcgctgagggtccgtcccctttactggggctggcagcagggtctcccatctcctctctcgggggccactgcagactttttagagaacgccttgcctccccccaaccccacccatccggggttccctctctccatcctctgcagtgtctcccatacccccattcagggtagccttgctattctccccaactccaggtcccccttcatctattccggggctggccgcggagtttcctgagcgctctccaagtgggtcctctagatgttaggagaacactgtacctcccccggtcaggggtctcctgtctccgttctatggagcgtccatgctcccattcaggactgccttgctccctcctctgttccggggctggctgcacagtctctgcaccccctatcctgaaagcctctcttaactatttggaaagcctcgtgtcctgtctcatacagggatcccctcatcctaatgactgcaatcttccattgctccatcccgagggcatcctgcccctattcccatcaggtttctccttgtcctctccctgtttcaagtcccctttcttattccgaacacactcgcaggctcttccgacgcgcacccgggggtcctcactggcccactccgggagtcctctgcccgcttccccgacctcgagggtctcctctgacgcagcgtcgattccccttccctcctcggtcccctgccccgcccctctcactgcggcggagccggtcggccggggggccgcaggggaggaggcggagagggcggggccctcctccccaccctctcactgccaaggggttggacccggccgcggcggctataaaagggccggcgccctggtgctgccgcagtgcctcccgccccgtcccggcctcgcgcacctgctcaggccatgatgagcttcggcggcgcggacgcgctgctgggcgccccgttcgcgccgctgcatggcggcggcagcctccactacgcgctagcccgaaagggtggcgcaggcgggacgcgctccgccgctggctcctccagcggcttccactcgtggacacggacgtccgtgagctccgtgtccgcctcgcccagccgcttccgtggcgcaggcgccgcctcaagcaccgactcgctggacacgctgagcaacgggccggagggctgcatggtggcggtggccacctcacgcagtgagaaggagcagctgcaggcgctgaacgaccgcttcgccgggtacatcgacaaggtgcggcagctggaggcgcacaaccgcagcctggagggcgaggctgcggcgctgcggcagcagcaggcgggccgctccgctatgggcgagctgtacgagcgcgaggtccgcgagatgcgcggcgcggtgctgcgcctgggcgcggcgcgcggtcagctacgcctggagcaggagcacctgctcgaggacatcgcgcacgtgcgccagcgcctagacgacgaggcccggcagcgagaggaggccgaggcggcggcccgcgcgctggcgcgcttcgcgcaggaggccgaggcggcgcgcgtggacctgcagaagaaggcgcaggcgctgcaggaggagtgcggctacctgcggcgccaccaccaggaagaggtgggcgagctgctcggccagatccagggctccggcgccgcgcaggcgcagatgcaggccgagacgcgcgacgccctgaagtgcgacgtgacgtcggcgctgcgcgagattcgcgcgcagcttgaaggccacgcggtgcagagcacgctgcagtccgaggagtggttccgaggtacgcaggcgcgcgggtggggggaggggcgcccctgctgaccccg

SEQ ID NO: 14 - DNA, 6000 bp, Artificial sequence
  A4GALT
agtaattgacagaacctcccctcccgtgtaagcagtggtctagatgttgacatcctgcctttcttctctagggcccaggaaatgcctgttgaaataaattgctggaaagccaccacaccacgaaccgtgcaaattgtcagactgttctttcccatgtggatatgaaatctgctttggagcatgggcccagtggggctgcagggagcccaccaccttccaggaccactactgtcttagccaggactaagagttatggactgagacctattaactgagggcccttaggcaaggctgtgcctggctccttaagccccagggtcctcctgggtaaggggtgcccctggaaagggcttagcaagtggtaggagttcaccccaacagtaactatgccagtgctttatccttcccttattgcaggtagaccaaagtggccacaggctttctattcaacattcacaaccagatgaccagctccagtttactgatgggagagaaacccattggttcaaagagtttaagaatttcagcctagctgggcgcggtggctcacgcctgtaatcccagcactttgagaggccgaggcgggcggatcacaaggtcaggagttcgagatcagtctggccaacacagcgaaaccccgtctctactaaaaatacaaaaaaaaaaaaaaaattagccggacgtggtggccggtgcctgtagtcccagctacttgggaggctgaggcaggaaaatgacgtgaacccgggaggcagagcttgcagtgagctgagatcgcgccactgcactgcacttcagcctgggcgacaaagtgagacgccgtctcaaaaaaaaaagaaattcagcctagaagctgggcgaggtggctcacacctgtaatcccagcactttgggaggccaaagcgggaggatcattagaggtcaggtgatcaagaccagcctgaccaacgtggtgaaaccctgtctctactaaaaatataaaaaaattagccaggtgtggtggcgggcacctgtaatcccagctactcgtgaggctgaggcaggagaatcacttgaactcaggaggcagaggttgcagtgagccgagattgcgccattgcactccagcctgggtgacaagagcaaaactccatctcaaaaaaaaaaaaaaagagccaggcattgtggtgcacacctgtggtcccagctacccaggaggctgaggcaggaggatcacttgtgcctgggaggtcaaggctgcagtgagttggaatcacatcactgcactccatcctgggcaacagagtgagaccctgtctctaaataataataataataataataaatcccagcctgagtaagagtgtctcaaaaaaagaaagaaaaaaaaatctgcccacacccaccctgctaagagaggcagattcaaccccaggcagatcactctcttctttcacaagcatcccagccctctccatctccaaccagaaagccagccacgagccatgccttcccttcctctcagagcctgtgtgctcaaccttaggaggaaagggacctgccagggtgctaggagggagggaagaggggagcactgggctcagaggcggagggggtgctccgggggtcaggagagcccagggcaacatgggccaggctgtgcttccagggcagagtttcacagatgagtgaactgaggccagggcaggtcagacacataggctcttttgtcccctgcttcctccttcacgccctggcctctgaagctctccctgcagtcgggacaggtcaggctagaaaataatttggcctcaaaggctgctggggctttgggggtcaggcaaatttgaattccaacctctgcttgttttttcctttcttacctttattttgagataggatcattttatgttgcccaggctggtcttgaactcctgggttcaaatgatcctactgcctcagcctctgtgagcttaggttaatttccatcttgggctcagtatttttcttttttctgggacggggtctcgctctcacctaggctggagtgcagtggcatgatctcggctcactgcaatctctgcctcccaggttcgagcaattctgcctcagcctcctgagtagcagggatgacaggcaggcaccaccacatctggctaatttttgtatttttagtagagatgggattttgccatgttggccaggctggtcttgaacacccgagcttaggtgatccgcatgcctcggccacccaagtgctgggattacaggcttaaaccactgcacccagccaacagcctccgtttttttttgtttgtttgtttttgagatgaagtctcgctctgtcgccatactggagtgcagtggcatgatctcagctcactgcaacctctgcctcccaggttcgagtgattctcctgcctcagcctcccgaggagctgggactacaggggcacgccgccacgcccagctaatttttgtatttttagtagagacggggtttcaccatgttggccaggatggtctcgatctcctgacctcatgatccgcccgccttggcctgccaaagtgctgggattacaggcgtgagccattgcgcctggcccagcctccgtttttttcatctgtaaaatggggataacagggtgacctcagagggttgtgagattaacaagctgcaaggagaccgtttccccagacatagccaggaagagcttgtgggtctgggcacagctccagcgatggctattctaagaaagccattcccttctcaattctctttaatccctttgcttaagtccaggggcaagtctttgctgggtgctggcctgccttgaatatctctctacacctttcgaatcttccttttttttctttttttgagacggagtctcactctgtcgcccaggctggagtgcagtggcgcgatctcggctcactgcaagctccgcctcccgggttcacgccattctcctgcctcagcctcctgagtagctgggactacaggtgcccgccaccacgcctggctgatttttttgtatttttagtagagacggggtttcaccttgttagccaggatggtctcgatctcctgcccttgtgatctgcccgcctcggtctcccaaagtgctgggatgaatcttccttttaacaaggagagggcaggaggaactcagctcttggaggggcaacaggacctacccgggctttataacattgttttagttttatttttactgttgccttctagtctctgagatactggtgttccatttagggtagcaatacaaagtttctctgaaaaatacatttgtgatcatggaaaagccaacataaagcaaaagcattaaaagtattacaagtgggccaggtgccgtggctcacatctgtaatcaccacactgtgggaggctgaggcgggtgaatcacttgaggtcaggagttcaagaccagcctggccaacatggtgaaaccctgtctctactaaaaatacaaaaattagccatggttgtgcatgcctgtagtcccagctactcaggaggctgaggcaggagaatcacttgaaaccaggagacagaggtttcagtgagccgagatggtgtcactgcactccagcctaggtaacagagtgagtctcttctcaaaaaaaaaaaaaaaaaaaaaaaaaaatgagtattaccagtggagaaagggtccgcattcattctttttttttttttttttttttttttttttgagacagagtctggctcttgtcacccaggctggagtgcagtggcatgatctaggctcactgcaacctccacctcctgggttcaagtgagcatgtccagctaatttttgtattttgtagagatggagtttcaccatgttggccaggctggtcttgaacttctgatctcaggtgacctgcttgctttggcctcccaaagtgctgggattacaggcgtgagtgcttgcctggcctcttgtttccgttttaagatgggagaagtaacagcctgtgatgggaatgacccagggagaatggagagagggcagggtttcgggatcagtgttggcgtgaagggtcgggatcttctggagggggtggtcgtggccaggagctggacgggttcagcccggtcccaagagggaagggactgggtgctacctcagcgggtgggtgggggagctggtgggaattttctactgattccttcattcgaagtgtatttattgggcacccattgagtgccaggctctttctcagggctaggatagaggttccattttctcagtggcttgggcactgggtcatcagctgagtgagaggatgggcgtcagcactcaagaagtcacccaatggtttcattttttgcagtctttgttgtcccagaggagtaagaactaaaagcaccaggactgtgacatgctggaaacatggcatgttccaaagagaataaaatacagttttatgatacacgaattgtgtgtgatacattccaatttctcacattgaacaaattaccaatagcaatatgtgtgaagatggggcgaggtgagagtgaggaggaggagtagagaaaggacggggcggcgttaaggatacagcaaataactggaattccgcaaacactggggtgatgcaggcaatgcccttagcacggtcccggcaggaggcggtgggatcaggccgacccgggtcccaggggtgacagcgtcccttctccactgcagaatatcgaggtactcaaccctctgaacctcagttttctcatcagttcaatggggaaaacgggatggtaacccgtaatactggcggcgcggaggccggggagcgctccctacctgttggccggcggactggggactgtccgcacccgccccggggagcagcgagggcgcgcgggcggggtcgcggggaccccgcaagggctctggggaccgggacccgcagggtaggtcgggacgggcggggcggggcggcctgaccccgccccgggccggaggggcggtgctgcctcccgccgggccccaggcactgccctccggccgccgcgccgcccgcccgccggggccccgctgtccgccgcccgccgccgctggagctagaggtacgaggggccgcgccgatgttgcgggggacgggggctccagggggatcctgcggctcgcagtggggaggaggcgcctcgggaaggcaggggcaggggcggaggggctgggccgccgccctagcccgggccacctgttctggaggcgacatttgtgcgcgcacgaaccccgcgcggcggtcccggccaccgcctccaccccaactgcgccccgaagtggagcgcggcggcgggacccggagccccggccctcgcgggcaggacgcgcctggctccggagccctcgagggcagccccacccggttgggtcccggggctggagagagggcgtcggggagaagccgggttcgaggcaggctctgcccggccaccgtgggtgagtcgcgctcctccgtccgggggaagcctgggcgtcgggcggtgctcgccccggagcggatgcgaccggggacggggatggagagcggccggcaggagggggctctgccgggctggcagcgtcggcgccggccttagggagggtgaggatttggacctgcccagaagcgggaagggccctccccgtgcgggaaacggcctgggtacgagctgggcacggggcgagccggttggagtcgcccggcctccgcgctcgcctcggatccgcaggcgcccctccctcctctccagctggggaaggttgaggactgagcgggagcggaactgggcttgggagaggatctgccggggcacctggtcccagcgcccatcccgtttcactgccagggcgagtccccttccctcagtttcatcacctgtcatccggagggacaagttcccacctggatagtctcggctgagcctctggccctgagtgggttaaagcgccatttcccctgcacccacctttccctcacgacaccctctctgcggcttcctcccctccg

SEQ ID NO: 15 - DNA, 6000 bp, Artificial sequence
  POU4F2
ctctgggcgagagagcgactcatttaaagcaggagaggggagcttggggctcaaggggagccagtgacaggatagtagttgacatactcagaagagaaaagatgtttgaacaaaacccacccatcattcctcaaacataaacccctatctcaatactcaagcccccaagcgccctccttcacctgaactttgctctgcaactacatccctgggagcttccagaagtttgtttcaggaataatccctcttgtgtcttctttttcctccctgtaacagtagaggccacggaagagtttaatctatgccaccccgccacccaaactcttctgtctcaagccacgagtccagagagagctcagggtgttcatcttctattcagaatctgaagcagattggctgattttgaaatccgtacaaaaacagatgggggaatccctcctttccctctttcttccaccaatcactctctccctgagatcgaaatggtgagcgaatagggccagatctgtcttttcagaaatcctccttgtagtccaattcaattctgtttgaaatacaaagaaatcaccctgcccttaatagattaaaatttaataaaagacattaggtccggtaaaatatgaatgcactacttaaaattttaatagtaaattaggacccagatacaaggaggagacagagatttctccccttgggattgcttcaggggcgggctgattttcgggtgggggacgcctactgcggggaccctttgcccagaagcctgaggggaatctccagctactcctcctcacccaggggtgggggatagtgaggggggctgctcacatctgtctgcatctgccagaggactggacagaagctattgtcaaacaaagaggcttggcgagaagaaaggagcgcctcctgaagtcacagagttgatccattttccatcttgctgcttaaaaaataaaatgtttattttctttttgctaggtaggtctgtaggtgtgtagatgcacaagtgtgaatagcggcttgggcataggtatgttgaggtcgtaggaagcaggtttcaaattttgggtcatttgcctccaccccttgggtttattgccaactttctaattgtttagatggctcctgataactgcggggctggaggttttcttccttggagagaggcttccacactcgctggagcactttgcagcaatgcctgtgggcaaaaccgaaatgggttttgttgatatcaccgcaacgatgttggtattctggacactcctgaaaggagagactgcaagatttcaagtcctggttgatgaaggaacaattgctttttccctctaatgcaacacaatcactattcatttttcactttgagtggggaatggagaagcctaactctatgacttgactttttaaaaatgtatgtgttttctaggaggaaggaaatacaggtaaataaactcttgaattgctctacaaccacattaatttacttcaaattgataatttgaaaacacagccttcctttttttcttgtctggaattagggatatcactgagaaatatcagagagataaaaaagttatgttaaattttttttgatgatatatgaatttcagttatcaggaaaatattctcatgggagttttcttgcttaaaatagttttggtcaataaattccatatcaaacaaagtttgtccagtcatttcaacagtgtccctctctatttcatgaatttattatagtaccgtatttaccatatgaagtggtgacaagtgtttagcattttggagatcagttgtcagttactattaaaatcaagattaattcatattaacgatatgcatttctcaatactcccaggtgacaacatacattaaaacaactattgcaagtaattaagctcaatgaagggggaggggacagagggaggttcaaaacaccccaaatatttgattttcaattcaagttcagcagactgttgccacaataatgctcgggaaactcctcagggttactttttactttacatctaattaggcacctaatgaaagagaagaattttttccccatgtggcttttctccttgatattcttgtgcctttatttaaacacataaacacactatttaggagtggcaatacccaaaagtttctcatgtgattaacttgctataataatctgggggaattagaaagaagaggacatttgtcttccggttattttccatcttcactctcactttggccctttggcctttacccagtacggatagaaaatacacccaaaatatttttatggatttttttattaggctttaaagatgctcaacagttaatactaattagaccatccaaagccttttgaagattaagcaaattggacaacccttgttaattagaacataatttgaagtttgaaattatatcactgatgaagtagtctaattagtatgacgatatgttaatggaccagtgagacatagtgccgatagagtttggcatgaacctcttcacggcttgcaggaaatcctccctcctataccgacattaataaaagatttctatagacttcagcctttccacgatgacatacaatttatagtacacacaatgcattagctcatagtactgctcaattttttcactattatgaaaactaataaattcgtcaagctgaattaagcatacaaatcagatgaggcatagcagattacacagtgaagcgcggtgtctcccgagcctaaatgaaatttcaatctaataattccttcctggcccagtcataatttgtttagagatgttgttctacttctttcaaagcgctattcgcactataattaaatgatactcaagcttttaactttgatttatttcatttcttgaagcttgagacagagctgtacaatgtcattttttttttgtttccttgaaaattactctggctgttgttgaggtagaaattaaacacctaagcacttacttgaaccgtccggcacaagccacattcattcacgtgaacactcccctttccctaccccatgtccaggtttcgctgagctcacacccggcaacactgctgctaggagttcccttcggctactatttattattttcctccacacaggggaagagaaagggaagcccgagaggatccagggaaagcagaagggggttaaggaccatggacagagcccgtcgcgcgctcgttgctgccgccttccccagcactctggcggctcctgaggacagcggtcccatcttgaaaccgctattccgcccggctgaggtcaggggtggacaggcggtcccctactctccaccgccgcttccgggagctgaccacccgagggttccccttttccactctccttcccactctgtttttgtcccagcgcgcgccagcgcctctcaggcctgccgcctgctctcgcacctgctcgccttccccaggcgcccagtgcctgcacctgctcccggtcaacccccgtccggattgggccacccgcgggttcctgcgtcggggtcccggggccttctcaccctcgcctgcaccctgctccttccgctctctagggaggtgacagcagcccccaacaccgcgggaagtatagagaaaatgggatccagaaggagaggaagtagtgtgtgtgtgtgtgtgtgtgtgtgtgtgtgtgtgtgacagagagagagagatagatagaaagagattatctccttttgcaactggaaccaagagtgtgtgtccatctctaggaaaagtggtctgcactgggactgggacagaagtgggagtgaagtgtcagctaaaaataggctccgcaccgagaggctgtggaaatgaagataagtgaggtttgtgccagcccccgagggtgtgtgtgtgtgtgtctgtgttgtggggtgtattcagcagcatatgcgctgtgtaatttctgaccttccctctccctgtcagttgccccttcttcctttgattgtggctaatgaagaataataaatccaggggcagggtttgccagtggatccttccaagactcaactcgaactgtactggatacagggaggaggaggaagagaaaaggggggcaagaggagcgtgtgtgtgtgcctgtgtgtatgtgtgtgtgtgttgtgggaggggtggggacagcggggagggggaggagtcgcatgcgcacagacgacccgagcctgctccgcggctgtccaatccgctgagagctgcgagaaatcgagtgagagaaagccctgcagcccctccgaccccatgtctctttggcaccaggcacccgccgggccgtggggggctcgtagccgaacgccgacctccgctcgtattgggctgggagttcagagccgcgcgcagaacccgggttggccgcaacgtctgtgttctcagcggtggccgggaacctgggatcagggtcacctgagctgacggggtgggggcgggccgagtggggttggaagcctggaacttagtggtaagcaggaggcgtaggaggtggcagccaggtaagaggcactcttacctacccaacgctggcttgggccgcaactttatttgggagtttctttttccggtgagacagagacccggcagaagaagcgggaggggctggaggctggtccttaggtaggcactgcccggcgactggagcgcggacctggccatttgggtggggttgagtgggggcgcgattgtgagtagcagccgcgggacgctgcgaaggggcggcggcaacagagcacgggcgggggcagaaaagaggcggcggagggcgcggtgggggagcgcgaggcgagtgctgagagagcagaaaggactcaagcctgaggggagtagagaggaagaaggggcaacgcgagaaaccgaacaggagccggcgtttcctggcaagggagggcggaggcgcgcgggagagagggagagagggagggcggggggcgcgggggtaggcgcggggagaggggagtataactcgccggccgcgaggagcgggggcagtttcgggtgccgaggtctgcagctagcggcaagcggagtcaggcatccgttcagactgacagcagaggcggcgaaggagcgcgtagccgagatcaggcgtacagagtccggaggcggcggcgggtgagctcaacttcgcacagcccttcccagctccagccccggctggcccggcacttctcggagggtcccggcagccgggaccagtgagtgcctctacggaccagcgccccggcgggcgggaagatgatgatgatgtccctgaacagcaagcaggcgtttagcatgccgcacggcggcagcctgcacgtggagcccaagtactcggcactgcacagcacctcgccgggctcctcggctcccatcgcgccctcggccagctcccccagcagctcgagcaacgctggtggtggcggcggcggcggcggcggcggcggcggcggcggaggccgaagcagcagctccagcagcagtggcagcagcggcggcgggggctcggaggctatgcggagagcctgtcttccaaccccaccggtgcgtatttctgcataatcaccgcttaaaggcacattttgacagccccctttatctgcttgatgtttttttcatgtctgcacagcaaatcaccccacacctccaaccaattttcccctctctctctcttaagtattcagcaggtcttgcctttcatattaatttttatgacctgggatgttgcctgtgcgcgtgttgtgttgtgtttcgttgtgtctacaggctcactttcctcctcctcctgcactctcggcttctttctgtggcttccctctttttctcttcacctctgttttcaggattattattattattattttaacgatctgggaatgttgtaggcgcggcgacggtgtcgagccctgggccggggcttccggagagagggcgtacaattccctgctgagcgtaatgtgtgccttctacttacaattgcagagcaatatattcggcgggctggatga

SEQ ID NO: 16 - DNA, 6000 bp, Artificial sequence
  C1QTNF3
gatttgtctaccaagagaatttgtttttcagcaaacatctataatacatgtagccagtttgagagttatatttttcattataaatgaatacattaagatgatagtttttgtacatgagtctctgaaagccttattcttgtgatggagacagatccattcctgggacactggcagtgagtttcctgcacttgaataatgaccctaaacatgtaattccaccaaaattgtaacatcctgaccagctgaccaacttcctctcttattcctttgttcctcattttgccctgtactggtggcaaggtgaacccatttttcttcaaatggcaatatctaatatctataggcaatgtcccctttctgaacacactctgattctttttaaaacttcttcttcaactggaatttctctgtagagcatctgaaaaagcccttaacatgaagtcataaacaaccaatacagctaaactacctcatctcaaggtgtatattaattttatttgtatattgataaatctctaattggtttaaatcaaagagactaagccttgattttagaatctcagaattcactgtgcttagtacggggagtatttttcactgggaagaggcaagagagaggtgggtcacatttccactgctctcgaaaatcatgcaaacagttgcctcctttagctgctctttaagttggcactgatacaaagacacgttcagtccaacacagttcttgactgtcccttggccagaccataactgatctccttttggcttaggccacagtgaagcaccatgggggaggccacggagtacctacacatcttataacggcctcctttgtaaagctaaacaaatatttccctctgccttgcaaagctgagtcacagatctggcttttacatgtatattcatatgcatctgcacctctgctgctcacattagtggccctgccaacgtcctaactgggcccttggggccaaggcaggcaggatgcagtgacagtcccagtgtatccaggagcctgatgtcctcaggtgtcttttgtgaactttcacagccagagacaactgtgcagaactgggacttcatcacctccagggaggttgcctggggcagattctagatggttttaatattcaacccataggtaaaaaattatgcccaattgttattcacatctttgaaaactagacataaggaacattctggagcatatatgaaatgataaccaagtaagatttaataataaaccaatgtttaaaatgcatttaatgagaaattactggagggaagtaaaagatacctatattgaaagatgagggactcttctctctggcccagttttctacttactgcccatgaacttaagtaatttactaaatcttttcagactgtcatctctgcgtgtacaagatagtacatgtgtagactccagacctctactgggtctggagtgtttttgttgttgttttgaaaagttttcatacatgttctccctatttgtttgctaagactgacatagcaaaatgctacaaatgagatgacttaaacagtgaaatttattgtgtcacagttttggagtctcaacgtctgagatcaaggtgttagcagggttggttccccctaagggttgtgagagaatcatctgttccacgcttctcacctaccttctggtggtctgctggcaatctttggtattccttggcttgtaaatgcattaccctcatccctgctttcattttcacatagtgttctcctgtgtgcatgtctgcctctgtgtctaagttctccctttttataaagacacagtcattttgggtcagggcccaccctaatgacctcatcttaagttgatcacctttaaagacctattcaaaagtaaactgaggcacaatacagttttaaagagtttgagcaaacagcaattcatgactcaggcatgagtctaaaccagaagaggttcaggagctctaccaagggagcacaaggggtggggaggcttctacaggacaaacacagatataaagcaaataaaatagttgattggttacagttgtacaattgccttatttggtctctcccattggaaagcttctgagtcatttaacttacattgtgtttttctttaatataggcatttacaaaaagttgctgaagttaagttttgcctatgtttgcaaaccaagcaaggttaaggccacttatgaaacctaattggctttgtctgctaagggattcttcaggcctggtgtctattttcatttactttaacaatccctatttttaaataggtcatatttgcaggtactagggattaggacttaagtttcttttgggggtacataatccaacccataatactttctgaccccaattcttgaaggcaggctccctggcagctctcagactgaccttcttctctctgccattgacccattattgatttcttgggagagatcaataatgggccaatggcagagagaaggagagatccttctttgcagaatccttctctgtagatctttctctatagcaaggcaggagaactcactctggaaagacagatggagtccagttctttccagctgcataacacaagacaagtcactttacctctgcaagcttctatcacctcatctataagatgggatcataggtttgccaacagaagtcagtgagataaagcaaagtagtacttggctagcacatggtaagtgcttaacaaattatggatattattatttattccttttgagtcaatgagaggatggatcagtgattcctaaccctggatgcaccttagaatcatctgagggaagcttttaaaaactgcccaggccccagtacagattctgactcaattggtctgggatagagtccaggcattaatatttttagaagctccttggtttaatatgcagccagggttgagaaccactagaatagatgacaggattgaaaacacgaatggaaagtctcaatgcggtagaaagtcagaggctgatttatagttacttagcagtatggcccacttcgtgggggatgcatgatggtgagagccagagacagctcttggaaaacctgtgaattgggtgacctagatttcagagccactgcttgctttgtggtctccctgtgacattttctctgggcctccctgaggagggaacacccccactgagacactgctctgggctttccttcccacagcaaaagcccatccacttgctcaccctccaaacacaggcgccatcctttccccagctccagctccaggtctgggaagaaaatcctcagttactaagaataacagtttgacacacttcccttcccagggacatctaccttttaatggtggacatgacagaactcaaggaatcctccagtgaggaaggtttagatgctcaagggtagacttgtagcacagagatggctgaatttttatgccctactaggtgggaactgactgcttggactgaacatgactcccaaggcccctgaactgtggcctggagagcttttagttcacagagtaactctctccctccgtccccagcaccaaaccctttccatgtataagtgtggactctggaagcggctgttctggtagctgcaggaggggccaggctagttttgacagttctggccacttccagagatggctcttggttctcaggcccttggctgagcaatgggggcagctgtcaacagctctctctcctctcccctttccccagctgctctgactcttcatcagcaggctgagcttctccaagcctcagtttcccacagtgcctcctcgcccccttctcatcagacgcttgccacccatgctatttacggtcctgggtctctccagttttcatgagagaaggccagtaacattttcttaagcgagtgaatgagtaatataaataaaatcatatgtaaatagaagcaatttttgatgacaaaactgtttaaattctcttaatttattatcctgaagcaattaggaatgtccattttctttttggctgaattaataaaagtatagcaactaaagcaaagacatgagaatcctcttaatctggatacgctgtgtttgaaataacgtgtttggtgctttccacttcttcagttttgttatttatttattttttaagtagagacagggtctcactaagttgcccaggctggcctcgaatttctgggctcaagggatctacctgtcttgggctctcaaggtgctgggattacaggcatgagccactgtgcccggccccgtttctattttaatcaagaatatgtcaaaagaaagaggagccagagacagccttctcttaacacaaagcagggtgttgtatcttcatctgacaataagtgacttagttaactttgatttaattttggcttttgggaagtcatggggaaaagaagtaaagctgagggtggcgaaatacctttgctaaacccagagacactgcacctgaatcctgctcagtgttttgtaggcaaccaactgccttctgccactggtatccggaaggggaaatgagttaccttaagtgggccaaagtggggaaatactaacagtacagctagaaaccaagagctgtcgtgaaagccctgaatgggttcccactgctatttcagacaaaactgatgcagttaagcctgaatttcctggcaagcacaagggtagatttttccaaaaggctttttagactgcaaatacacagcctttccatgtctaatcacaaaagcaaactgctggacacatgctctctgttcccagctggttgtgtatgttttcttcacacttcaacagaaacgtgccaaggtgtgacatttatgaatttcgccaactgggaactgtgcacacaaatcgttcttgcttgtggtgtgggaaaatgatgaatgggggctgttctgcctggggaggagggctgctgtcactctggcatgcctacagccttatttattacacaccaaagtataaaaccactccgccgctgcagctctcagctccagtcctggcatctgcccgaggagaccacgctcctggagctctgctgtcttctcagggagactctgaggctctgttgagaatcatgctttggaggcagctcatctattggcaactgctggctttgtttttcctccctttttgcctgtgtcaagatgaatacatggaggtgagcggaagaactaataaagtggtggcaagaatagtgcaaagccaccagcagactggccgtagcggctccaggagggagaaagtgagagagcggagccatcctaaaactgggactgtggataataacacttctacagacctaaaatccctgagaccagatgagctaccgcaccccgaggtagatgacctagcccagatcaccacattctggggccaggtactcagaattctcttctaaggatttttgtgaaagcttaatgagtgttgttgttgttgttttgttgttgttctttttttggctaagattcttagaatcaatgaatgctttcgagaagttcgctaagctgcttgtgaatctctttctggattgctcttgggtacaagagagactgtcggtctctgttagaaaatcagcagaggcagcaatgatgtgatgggatagtggccgtagcatcaccccatcagaaaggggaacctggccagccagcttcctgctatgctgggacttgatttccttcttgctctgttggtccggaaaggagtgctgacccatgcagacagatcggtcactgtagacaatcatttgttgttcttaagagaattgggctttcttcatcccttccggcagggagaagctgccctttgagtttgtcaaaagcaatcaaagtttttttactttgatttgtgagtatactgtgacatttcatggcagtcttgtttatttgatttatagcattctagattgttaagcctctctgttcagcctgttttaaaaagaattaaagagttaaataaaaaataaactgtttgaaatgcataccttatag

SEQ ID NO: 17 - DNA, 5458 bp, Artificial sequence
  HIST1H3C
ctgaccaacatggagaaacctcatctctaataaaaatacaaaattagccaggcgtggtggcgcttgccagtagtcccaattactggggaggctgaggcaggagaattgcttgaaccctggaggcggaggttgcggtgagccgagatcgcaccattgcactccagcctgggcaacaagtgtgaaactccgtctcaaaaaaaaaaaaaaaaaaaatcttagaaatgtaactgacatatcataagccctcaaacttaataatcttttaatacatggagctatctatttaaaataatgtacataaggcaacatcccaaaagaaaatgggcaagaatcatgagtaatcaaaccataatagaagaaatgttattatcaaaatgtgcagtctcaaacaataattgtcttaaaaataaaaacaacaatgagatttaattgttcatgtcggcaatttgaacagactaacacacccactgttcaagagcatttgtggaagtcaggaaaaaacaccctgttggtgagagtgtaaacagaccttcaggaggcaacttggtaacatgtattaaaaatcaaaatatgtatatcaatggatgcatgattcctatctctatttttgcccttacagcaatcttgtgtgtagagaaatactgaaaagcattttcatggtaacatggtttaaatttttaaaaagcgaaggtcagtgaataaagggcaattatctacttccctacaatgaaatgcagtaatgaaaataatcattagaatctcttttattaatttaaaaggatactagaaaagtgaaatacaatctcacttatagaagatttacatattggtttgcatagacttgcacaagataaaatttctgtaagattggtcaccaaaatgtcctgaatgataacattacaattaatgtttatattgtaggggaaaagaaaattctgtttttctcacccatcagtaagttcatgcttgaggcccctctacaaaaagacagattggtcgggtgcagtggctcacgtctgtaatccgagcactttggcaggacgaggcgggcggatcacgaggtaaggagattgagaacatcctggccaacacggtgaaaccctgtctctactaaaaatacaaaaattagcggggcatggtggcacgtatctgtggtcccagctactcgggagggcgaggcagtagaatcgcttgaacctgggaagcggaggttgcagtgagccgagatcgcgccattgcactccagcctgggtgacagagcaaggctcagtctcaaaaaacaaaaaaaaagattagcaagagaaaagcatacaaatgtatttaatataagttttatattacatgggacccttcggaaatgaaaactcgagggaagcgggaaacctgtgaatttttatggcaagttttgtgaaatgcatagttgtggattaatatgattgacagtaggcatatgatctaatggtaataaactgagggggacatagcaaggcttgtttgttaattacctattaacgatcagccgagtatcagcagagacagcaaaacatcctagttttgagttagaagacctaggtttttgttttggcttatcaattatgggtattgttttagatgaaacatcaagtattcttgatttcttatttcaaaaataaaaaataaaaaataaaggaaggaaaaaagaagaaaaaaagagaagaaaagtgtcagagttacttgaaccagagtaactccattttgagtgagggctaggaaaatgaggctgagactttctgggctgcattcccagaaagtcagtcattcctagcttctagatgtttacggttaagggaacaaataaataatgtttactaaacagactcagacttaggagtgtccagatatccctatatctggagaacaaaggcattcttaattttgtttaaagataataatgttgattcttgcaaaatatagtaactaagaaaattaatcctttatcacaaacttgtagcagagcacatctccccatatatacaagtattgtacctagggtggatgccttcctcctcttactttcgggaatgtcctgctccgtctatggagtagttgtcgtttcaccactttactttcttagtaaacttgcatttactttgcactgcggactcaccctgaactctttcttgcgcgggatccaagaaccctctcttggggtctggatggggacctctttcctgtaacatatttctggccaccacagaagggactatagtacagaaaccctgacccaacagctacctttgggtaagtgttggagttctgtaacaaaggaagaaggcaggcaggcaaaaaatttatgaaagaacatacgacaaaataatttctgcttcaaaacttcatatttttttaatttttttttttttttttttttgagacggagtctcgctctgtcacccaggctggagtgccatggcgcgatctcggctcactgcaagctccgcctcccgcgttcacgccattctcctgcctcagcctcccgagtagctgggactacaggcgcccaccatcacgcccagctaattattttgtatttttagtagagacggggtttcatcgtgttaagcaggatggtctccatctcctgacctcgtgatccgcccgcctcggcctcccaaattgccgggattaaaggcaagaggcaccgcgcacggccccgtccaagttaaccttggctctaaaacttgtcttcgctaacattccagttgatcctctagaactgaaacagaatagcagcagcaccaccttaagaaattgtggttatagctctccttgtgacaaagtaggtggctctgaaaagagcctttgggtttggaagtgcttacataagcacttatttagagctagtgtacttggtaactgccttagtgccctcggacacagcatgcttagccagctccccaggcagcagcaggcgcacagccgtctgaatctccctggaggtgatggtcgagcgcttattgtagtgagccaggcgagaagcctcgcccgcgatgcgctcgaagatgtcgttgacgaaggaattcatgatccccatggccttggatgagatgccggtgtcggggtggacctgcttcagaaccttgtacacatagatagaatagctctccttgcggctgcgcttacgcttcttaccatccttcttctgcgccttagtgatagccttcttagaaccctttttaggggctggagcagacttagagggttcaggcattgctattcctaaacagaatagaaaagctactaacactctccactacagagtagtacagagaacagttcagagcccatgtatttatagtcctgagattcaaatgacggtttaagattcctcacttctgattggacaaaagaaacacggtttcactgaggggtggggtttatgcaaatatggaatttatgttatctttttctattggataaagcaccaaacataattgaccaataggatagcttcctattgcagccttgcagtttgtataaaaggatttgttcaggcgccattccagcttgcttgtctttcacagttttccgctgctttcataggtcgctatttgcggacgtggaaaatggagctaaagcaaaaacttgttcgtcgctaccgggcttgcagttcccaatagggcagagtccgtcatctttttcgaaagggcaattattttgagccggtcggagccggtgcgccagtgtacttacaatacctggccgccgagatcttagaactggtgggcagcgccatacgtgacaagacccgcagcatcatcccccgccacctgcagctggccatccgaaacgacgaggaggtcaacaagcagctgggcaacgtcactattgctcagggaggcgtcctgtccaatattcaggccgtcctgttgccaaaataacagagccacgataaggccaaggtcaagtaaacactcaaatcagaaaacgtagcttacacttgaaacggcatttttcagagccgtccatagttacacaagaaaggatgataacttgcttctgttagggtattttttgcttttcgtttggattggtttgttttgagacagtctagttctgtcacccaggctggagtgcagcggcgcgatatcggcttactgcaacctccaccccgccgcttcacgcggttctcatgcctcagcctcctgtgtacttgggattacaggcgtctgctaccgcgcccagctagtttttgtatttttatgcgagacggggtttcaccattttagccagggttgtcttgaactcctggcctctagtgatcgtcccatctcgccctcccaaaatgctgggattacaggcgtgagccaccgcccccctagcctaatggtgttaaaaagttaagtttcgagaaaataacaccttcctttagaaagtacattttagagtatacaaagtgaaacttaaggccaaccaaaataagacattttgagaacaggcagggtgggaatgtgacttggacttagaaaacaaagggcaaggaaacttgctgttcgccagtaacaaaatagcatggaatctcattctctgaatataagcgttatttcccgacatgagtctgaacgtttctggtggtttagtgagtgttcaccagcattgataacttgcgagactgtcaggaatgcagaatttcaagtcccactcaaacttactgaatcggaatttacattttaaaaatccttagataccttgttatacactctgttctttgggactggatgaactagaattttagacaatttgtcgctgcagataactgaaacgaaaaggacaggatgggcggtggggcaactcatccaataagattgtctagtaatgaaccaatcagtctggtcactcttcagccaatgattttatcgcgcgggacttttgaaatattacaggaccaatcagaatgtttctcactatatttaaaggccacttgctctcagttcactacacttttgtgtgtgctctcattgcaaatggctcgtacgaagcaaacagctcgcaagtctaccggcggcaaagctccgcgcaagcagcttgctactaaagcagcccgtaagagcgctccggccaccggtggcgtgaagaaacctcatcgctaccgcccgggcaccgtggccttgcgcgaaatccgtcgctaccagaagtccaccgagctgctgatccggaagctgccgttccagcgcctggtgcgagaaatcgcccaggacttcaaaaccgacctgcgtttccagagctctgcggtgatggcgctgcaggaggcttgtgaggcctacctggtgggactcttcgaagacaccaatctgtgcgctattcacgctaaacgcgtcaccatcatgcccaaagatatccagctggcacgtcgcatccgtggggaaagggcataagtctgcccgtttcttcctcattgaaaaggctcttttcagagccactc

SEQ ID NO: 18 - DNA, 5439 bp, Artificial sequence
  HIST1H2AJ
gttgccctcaactcaaatattctttatgtcaatgtggcatattttggggtggtgtgtcttgccacccttcactcaaaaacagctaatctctacccaaaaagagaatcctggctgggcgcggtggctcacgcctgtaatcccagtactttgggaggccgaggcgggtggatcacgaggtcaggagattgagaccatcctgactaacacggtgaaaccccatctctactaaaaacacaaaaaattagccgggcatggtggcgggcccctgtagtcccagctactcgggaggctgaggaaggagaatggcgtgaactcgggaggtggagcttgcaatgagccgaaatcatgccactgcactccagtctggttgacagagcaagattccatctcaaaaaaaaaaaaaaaaaaaaaagagacagagaatccttcccctaaaaggagaccctaagtatcggcccttaccccttgcctagtgtgcctgcctttattcaaaatgtgagactcaaaacttattcgaatgttactcttttattgtctcacaataaatccaagcccataaccatttcttgtaggaattagtctttttgagacacttcacagtactcacaagcctaaagacagaaaactagatttttttttcttttcaaagaatttaacccttgcttaataataatcagctgtgacatttggatttaagtgtgaagttactaacaaacatagcactcctaggtagaatccacccaaccatgaccatgaacctccaagtcatagcttgaatgtacccattgcattgaacggcctctacttttttcccaaaagcccaaatgcctctgtgctatttctgtctcatttaaatttaagcttcttggccaggtgcagtggctcacgcctgtaatcctagcagtttgggaagtcgaggcaggcagatcacttgaggtcaggagtttgaaaccaggctggtcaacatggtgaaaccctgtctctactaaaaatacaaaaaaaattagctgggtatggtggcaggcacctgtaatctcagctactcgggaagctgaggcaagagaattgcttgaacccaggaggcggaggttgcagtgagccaagatcataccactgcactccagcctgggcaacacagcaagactttgtctcaaaaattaattaattaattaattaaagctttttttttgtttttatatccattttttttatttccttcataaagatgtacattatattacattgacacattatattcaccttattttgtagggtcaccattgacctaggcactatagggggaaaaaaaatgaaacccagtctgcactccacatgacttgtaaaaacaagatatagtcatcaacacctccaatattggcttacacaataggctataacagtgggttagagttctccaagtgcatggggaaagtaaaatcaattagtgtatgggtaaaacaactcaataaagaacttttaaatattgttttgctaagtattctgtgaccaaaatgtgaattttatctcttctaaaatgactgtacatacatataaagattgtgtcgaccccaaaattctgttttgttctattaaaaattaaaaataggggcagggcacagtggctcacacctgtaattccaacattttggaaggccaaggcaggaggatcacttgagcacagaagtttgagaacagcctaggcaacatggtgaaacactgtctccaccaaaaaagatacaaagattagcccagggtggtggcctgtgcctgtagtcccagcaacccaggaggctgaggtgggaggattgtttgagcttggggaagccaaggtgcagtgagccatgattgtgccactgcactccagcctgggcaacagagtgagaccttgtctcaaaaataataatcataaacaattttgccaagatttctggatagtattttcctaatttttttttttcttttagatgtagtcttgctctgtcaccaggctggagtgcggtggtgtgatctcagcgcactgcaacctctgcctcctggattcaagcaattctcctgcctcagcctcccaagaagctgggattacaggcgcataccaccacaccctggtaatctttgtatttttgtagagatgaggtttcaccatgttagccaggatagtctcaaacttctcacctcaagtaatctgcctgccttggcctcccaaatttctgggattacaggcatgagccaccgcactcagcctaattttttaaaaaaatcttttgagctttaatttgtttctaacatgatactcctgtatcaagaatagatgcttcatctaatgagattgtattgttcgggctcagaaaccgattccccaaaatatggagctttgacatactgaactgaagaagagtactcaaggtctttcggaccttccccctattcctctctctcatttctctatctgaaagcagagaatgaagttgttctctgaaattcccttatctctctaaagtatagacctgccaaagaagaaaacaattacctctggtctcttctctgagttttcattaactgaaaactcatatcgcaagaagactgaagtctgtcaacacacggagacaaacctttgccacaaatcattgtctggtctgtgggccaaacagactttgtcctaggctgttatgttattcaagcctattgaattcccctaaaaatcacttaatacccctgtaaaatcatccacacttccccaactcccttttccctgagaagaagggtatgtaatcatctgtattctattgcatggggcgtgggggggagggggagggagtaatcactgattctcccccatgcacattaataaatttgtatgccttttctcctattaatctgccttttgtgagttgacttttcaccaaaccttcagaggacaaaggggaagttttcctttggattctacagtttcaatagacaaccaaaagttaaagttaaagttgggaattatttaaatatgcttcactctttgaatgtattattcttacttaatctattaacatgtatatgtctttgctaacgttttgataatttattgaatggaatcctaaattggaaattcctagcataaatcacacatatgttagaaagtatttttcaggttgggtttctttaaagaagtgtgaggattcaaaggctctggaaaagcaatctcagtgcagatgccttttgaatccttccaggtatctgagttttctcacaattttaaaaattgatttaaacataactagaatatttctggcaatttaactgtaacacatctacagaacactcactaggtattcacagtgatattaagagcaattattttttccagttcattttctttgatttacctgattttttttgagacagagtcttgctctgttgcccaggttagtgcagtggcgcgatcacgactcggttcactgcaacctctgcctcccgggttcaagcgattctcctgcctcagcctcccgagaagctggtgttacaggcacgtgccactgcacccgtttaattttttgtatttttagtacagacggggtttcaccacgttggccaggctagtctcaaactcctgatctcaagtgatccgcccgcctctgactcccagagtgctgggattacaggcgtgagccaccacgcctggactaaccctccacatatttatttatgactttacctataacttctgcttccctaaaatgtacaaaacagttgcattctgactgcctcagaaccactttctcatggtctcgggggattgtgtcttccctaagccacggtcactcatagttgctaataatcctctttaaaatattttgggccgggcgcagtggctcacacctgtgatcccaacactgggaggccgaggcaagtggaccacctaaggtcaggagttcgataccagtctggccaacgtggtgaaaccccgtatctactaaaaatacaaaaagtagcagggcctggtgccacatgcctgtggtcccacctactcgagaggctgaggcagaagaatcgcttgaacccggaaggtggagattgcagtgaaccgagatcgtgccattgcactccagggtgggcaacaaagtgagactacacctcaaaaacaacaacaaaaaacaaaaaaaaacccacatatacaagtacatttatatgtacgaagcgagtcccaaagggtacctaatgggagacaaacttaacagtgaactggctctttcttgagaaacgtggacggctctgaaaagagcctttggggtgtgggtcacggcggaactgttactgcagcgagaggctcacttggagctggtatacttggtgacggccttggtgccctcggacacggcgtgcttggccaattccccgggtagcagtaggcgcacggccgtctggatctccctcgaagtgatggtcgagcgcttgttgtaatgcgccaggcgtgacgcttctccggcgatacgctcaaagatgtcgttgacgaaggagttcatgattcccatagccttggaagagatgccggtgtcggggtggacctgcttcagcaccttgtacacatacacagagtagctctccttgcggctgcgtttgcgcttctttccatccttcttctgagccttgttaatggccttcttggagccttttttagggactggagcagatttgactggttcaggcatggtggaaaacaaaataaaagacaaccttagggctgtttcgtcctctttatttaaatgttattatgcaaattaggagtagaataggtcagtgctgattggtgattatccgtggatgacgtcagatgccagttttgcccaatcaaaataggtatcctgcatactcgagtcctattggtctaaataaaaataaaacgtaagccaatcgcacagcttccttttcgcgcccagtagaggctataaaatgtacgtttttccaatttcatttcagtctttcttgaccgtaaaggtaatagaccttttgccatgtctgggcgtggtaagcagggaggcaaagctcgcgccaaggccaagacccgctcttctcgggccgggcttcagtttcccgtaggccgagtgcatcgcctgctccgcaaaggcaactatgcggagcgggtcggtgctggagcgccggtgtacctggcggcggtgctggagtacctgaccgccgagatcctggagctggctggcaacgcggcccgcgacaacaagaagactcgcatcatcccgcgtcacctccagctggccatccgcaacgatgaggagctcaacaagcttctgggcaaagtcaccatcgcacagggtggcgtcctgcccaacatccaggccgtgctgctgccaaagaaaactgagagccaccacaagactaagtaaagaccgagttgaaaagcgcataaaaacaaaggctcttttcagagccacttca

SEQ ID NO: 19 - DNA, 6000 bp, Artificial sequence
  MLN
tccccaggttgtgtgcacactgtcaatagtccttgaggctgaacgaaaaagaaaagcattttattggattgtttcgggtccaggttgtcagtgcacattgaatggactcaacaggaaattggagatgcaggaatgttaattcaagtagcagatgtaacacacaaacaagaagctcggaaaattggaaattaatcctggccagaccctctctgtgtcagtgttttcctctccacttgcactgagtctgtatcatggggctcttggcgtatggtttctgctgtgtgtcatttctcaacaatcatttgtgagttctgaaaaaagatcctgagccatagaaggaaaagaataaaaatactccacaggtcaccaaagcctgtgtcccctgggccaggacaaagtcgttgccaaaatacatctctcagagctttgcttgacccagttttaaacgtcgtcaccatcaactcgtaattttgcttatgagtccagttgcacacctctggctctgtaagggacatcaaggactttagatgcgagggctcagcaacaactctctctcctcctgccctccctcctacacctgggttctttgccttgctttagcctttgcttaacatgagggatgcacatttaattgttaccagcgttctggacaagccaaccttttgaaacctttcatttttcattgttattccttcagctttggcactcctgcaatccttaggctgttttggctcccctgaactctgcctttgtctggccgctgccctctggctcattgaggaacccacctggagaacagggtggtgttcaagctgccccgcccgcaggagcccccagttgcaccttttcttcttggctggtccctccctgtcacccatatcctccatcctgtgtcctcggggccttggtgtcatatgtccagcagcccacctggccatttccagcacgcagttctaactcccgacatcttcttgtattagatctctgtcctccctgatgtttgctatatctcccaatttagtatcatctgcaaatttaattaacatgtcgttgcctccctgttccagaccgttaatgaagatgttatggcggtacctggcaaacaatattttcaaggtcatgaggagaggagagctaaactgctaatacctctgcctgctgtgggagaaagaagcaatcaagtgctcacatgtggtttgagccatttcctctgagttttgtcaacttcctttcccagattggggttaggtttctacgcctcccctctgcctcggctgctgcatatccgtggcaattcccacttggaaaatgaagaacatcagattatgccttccttttatttttgtctggtttctagcaggagagaaacagccactaattgagcatttgtagggagagccaaggtacatggtaaagacaaagtagtggttattaagcttgttatctacccagctccatttctaaagcactaatcgtggcagtggaaaatgcccaacgaggaccaaggcacaaacaggggagagaagacgtattcctggtgagatggttcccaaagggctggctcacacagcctgcagcccagaggagtgtgaggggaggccactgcatcccagcagcttgtgaccccaaagcagacctggagccccctgtgagtctacattccccttctgcattagcaggcatagctcattgtgaaccctggtttgccttccttccacaccaaactgcagatgtctcactgtaatactgcaaattacttgggtcagaaacactgaatcatgggagcgatccaggttagtgctattcacgcccagaaaattgatcccgggttatttactgtttcccgcacttcccttctccccatccctcactacaggcctgtcttgggcttggaggctcctaatcatttctcagacagagtgtcttggaggcatctctggaccagagcccagactggcccagcagaggcagggcacggatctcagacctccagtgtggaggggccctgggaaagtggcctcagatgtctccttgtgctcagtggataggcctggtccgccatctgcagtcctcctcccagcccaagcctggcatctccaagcctaaggacacacagcacaagcggcacttgttccggttggtcagctcaggttgcctcatgctcagagacctcgcaggggatggcttaaggagagagtgacaggtttgtagaattggtaccaggccactgttttgtgtcttccccagctataaacatccctatgcacattcaccaaacaatgaggggcaggagaacacagttatttaataaaaaaataaaaacttgattctgaagccagactccctgagtttgaatcctggcctggccatttgaatagctgtgtgaccttgggtccgttgttccacctcagtttccccacatgtaagttgggataataaccacatgaacctcagggctgttgtaaggttttaaaaagcagatacgtgtaacatctggctgttatttatcacatgctccagacactgttgaggtcaggaccagcaaacgttttccataaaggtccacacaacaaatatgttcagctttgtaagccataggtctctgctacagctacttaaactctgctgttacagccccaaagcagccataggtaacccagaaatgaatgagtggacctgttccaataaaactgtatttatgaacatggaaattaaatttgcatgtcactaaatattcttttgatattttaattattaaaaatataaaaacagtttttagctcacagactataaaaagaagtggcaggccggtttggcccattttgcctgtcctggtctaggtgctagagatgcaggtgtgatgtgtcccatcacagcagcccgtgcttatggatggagctccccagagcatcccagagcttgagaagctgatagactacttcccaactctcaagggactactttgcccccaccaaggtctgtgaccttgtctttcggtttggttttgttttttttttcagacacagggtctcactctgtttcccaggagtgcagtggcatgatcatggctcactctatcctccatctcccaggctcaaatgatcctcccacttcagcctgccgagtagctgagaccacagcgcatgccatcatatctagctaattgtttttattttttgcagagatgaggtcttgctatgtttcccaagttggtcttgaactcctgggccttaagcaatcctcctgcctcagcctcccaaaagctgggattacaggtgtgagccaccttgcccagcctagcgacctcttcccaaaagcccctcttgaaacagggacagtcagactccaatgaggcccaaggttaagggatggtaactctgctgcaaacccccggcatttggagctttaattcctgatgtaagtctcagacttcagagccacactgcacatctcacaacagccgctggtccagtgtgctggagttctaatgtgggtgctgtacacagagaccctgctggcaggaagcgaggacactgagctgccatctgttttcattaagccttgctgagtagggaaggtagaaattatctgtgttgggtttgaagcactgcaaacatttgtttctgagtggaagggccagagcagacccaaggactggctaggcagggtctcctgggcaagaaatagtgcccctctggatgtgtgaggcagcgtctggatgtgtggggaaggtgctactggccaacgtatgtcgcctgggaaaatggccactgcccacccagaccctcgggattccacacccagactgtaccccacctcagcctctgcctggtccatgaccggtacccagaaaactagacatcttccagagtgatgtttttgccctaacatccccttcaaggtatcatgaagatgcccaatattccatttcctccatcaaatctaaatccttctgcttggttgctaagggtccccacactcagacccgactgagcggctcccagcccacacaggggacctcctgtgacaccattctaagcctgtgtgtgccccaaatctccagaagccctcactcctgccctctgctgcacctggccacgtctggccttcaccctagtataaagacaccttgcagaggagtcacagatgagctatttagcagtgcctccgtttccttatctattgaatgggggaaataaatgcacccacctcacggggttgctgcgtttaatcagaacgttcgtgctcagcatttcctgggtaatgctgcgctctggaattggccccgcccagccccagcaacagcgagccaggtctgactccagatcacattcacctcttccctcttccctctttgaatctttacacatactgtttgaattgcatgattaccatgaaacattgtccaggtaggtgtctttctccccgtttaggctgcagcgtgcagacctcctacactgtcacccctctgcatttcctcggttccttcgcggcatctggccccacacagccttgggtctggactcagtagttttattttcattaatccactgaaatgctgtcacaaactcctgaacactttctttcatgaggggcttcagggggaagactgaagagaagttatctttctaatacagcgctctgacaggccgacagaaaatctgatcactagagcattggcctgcagacgctcaccaatgtcatatatttaaggaacaaaaaaagaaaaggctttgttaaaatgacctctgagaggcagctgagttttcagtggacgggagaatgccatctgggtgggggctagcttccaccgaaccctgactgtcgctgttccttccaggaaagccctggaagcccatagcgtggctagccctgcctggagtttccacgagcttcaagaatccaggctccccctctgagggcccccaaagctgtggtcaaaggttaatgggctccaagggcagctcccagggttgggaggtatataagaacccgtcagatcagccggacaccagaagacaagcagagagactcctccagacccactcagaccacgtgcacgccgtaagtagcccttggagaaagtgggtggggagtggtcagcataagccctaaagcagaacgctggtgcaagccagagccagcctggtccagggccctctgccaccttccagtgcccagccgggcttcgcactgagtgcccgcgctgattcccagggcatcagtgagcagaggcagggctgaggcacagacgctggaggcaagcaggtgggtacaactcctggcaaagcagaggctgttgtgggtgggtctggcacatccacggtgggccgggaacccaagccagagctcatcatgggcgcagggcccctcttggcatggtggctttgctctggaaaggtaagaaaaatttggccctgcagtggctcatgcctgtaatcccagcattttgggagactgaggtgggcggatcacaaggtcaggagtttgagaccagccaggccaatatggtgacaccctgtctctactaaaaatacaaaaattagccatgcgaggtggcacatgcctgtagtcccagctactcaggaagctgaggcagaagaatcgcttgaacctgggaggcagaggttgcagtgagccgagatcgcgccactgcactctagcctgggagacagagcgagactccatctcagaaaaaaaaaaaaaaaagaagaaaagaaaaggaaaagaaaaagttgaccccgagggagagcacatggggaggcaaggctagcccggccaggggtgctgcaagggagggcagacggtcacccccttcatgcagagctggacacttgaaggttgaagccccccatctctgatgatgggaaaggaaagttagtgcctcactgtacaatgaaaagctccttctcccacctccagctcaccagaacacacatgaacgtaggtgacatgccgactgccagttggatcaagaaaatgagaagcaattggattttg

SEQ ID NO: 20 - DNA, 6000 bp, Artificial sequence
  TWIST1
aataatttattatcctaaaatttgcattaaaatataagtggaaaactataaaacatagttttcatatagttttcaatgtaaagacaacccaaatgcctgaagtagaaattcaaactgttaatggcattttgaaaaatcttttagaaaaaaaaaataaactgtttttcattgccaaacatggaattaagtaaaattcaaagtcaccaagagttttctacaaaattatgtcaacgtaaaataaacattttcccttaactacattagtatatttagtatattttctttatgttctaagaaagtacaaattataaaatcttcaaaagtttgaatgctaactcatgtttttttaattaataaaaatggcataaatcaaatagacatctccaaatgatattcattaattgtgtgttatttttgctgttttgttagctttcctcttctctgagcttctttctaaacaaagttatctgggaaattgtgtcctcttgtggtgactggcaaaaacaacatgttttctgcagaaagaaccaaaccaaacaatagcagcaggacataaggcttcaggaaaacccatttgtgaaaaacggaaatcccttccagtgaagaaaggtgaaagttatgagtaatcagaaagagaaattgaattgaaaaaacatagaatgtaaatcattccatttatatttaaattactggtcatcggatgattaaaaagaagacacataatgctatgtagaaagactgagaccatgataatagttcctatgaataactatttatttcttgcattctttcatcagttattccagcaactctttgatacaagtgaagtgagtgtgttttccaacaaggttaatgggcagctaagacaccaactctccagcctgcacaacccctgaaaaaaaaaatgaaaaaaaaaaatgctcttcaagactccatacttttccatattctgatcctgcactttctacaggttagacctatgtctcctcatcccatccttaagctgagttcagaatgtcagctattttaaagattaacatttatttgaaaatttcacttgcggtactgttcgcaccctaatttattgtgttttttactttttcgttttatttctggattgaaaacttcacattttataactttagtttgttttttttacttttaaagatattcagatagaaaaatgtagaatcagttgagtttatgaatcaaatcatcatttaaagagaaagagaatgagtttttatatagggaaaggtgttcaaacatcattaaggctaccagggtcatgaaagaagaataagcttttacacagttattgtgttgacatacatgactttatattccaccttactaaaattctctcatttaaacagcagtccctctggagtgttcaaagcaagatgaaatctaagatatacaacctcatctgatattcactcaattgcagcctttctaattttcactcccttccgtgcgaatgctcctgtgcactatttatgcaattcagataaattatttctcagaactcggagccaacaaacaggtgataggccatctaaggtctgaggagaaagaagagtaggaacagattgaggtactcaccttttatgcaagcccaaactccagaatttaccagttggctaaccattctatagtgtgtgtttcttttccgtatctaaaagtatttataattggttgttgctaagagaatgtcacagcatattcaaactgccaggatacttaatacttattgtatatgttcaatgtgagatttctaactctgttacttttaacagtattttttgtttcaatttttaacacagcacacattcctcaggcaggattgcaaacatgccaagtttgcagtttgaaaacaaagatcgttttgaactatccaagtacaaaagaaattttttttttcagttaaatttttttggggaggactttactttagaaggaggcaaaatgactgaattccagttaatctagcattcccaaagaaggtagtccccactgacatacttccagttaggctaacttcagcctcaaaagaaaatttctaagattgttttcatgatctcaagattgggattttagaaagaagctttcaaatttaaactaaacattgcagaagttgcaattattgttgcatatataagagaatccagaattatacctgacctgtcaacaagtaaaaagggagccatccttgcaaatgtggaaaagaaaattgttaggtaaaatgttagataagaaagagctgtatatgagcagactggactcgtcagccagggcgtgttgcagtttgcaaagcagggagtatctagctccagccattttttgagaggtacctcctggactacttttgctccaaaagtaacattcaagccccacttaagaactcaaaagtcgtctcatcctccactccccggaacagttcaagagccatttcttcttagaactagtcatcaattgtgactgtggctgaaagagtccatgggcaggatggttctggttaggtgttcattttgaattcccctctcagtattctggcatcagagcgtcgcctgagccttgcggggatccctgtacccaatggccaggagctcttcatcagctagaagtttagtgccgggaaaagggctgggctcacttgtttaagaaccagtctttgagactgatgactttgcaagatggactggctaatgacccctggctgctgcgctgctgtggacttggtttctcctctagcttgtccgctcccctccccttcccaaattccccttggtcaggtaagatttcctttacactttacccacactttcctgtcttacttatccgtggccacaaaggaaagagtccaatcattcgatctctttatttatttttgagaaaagagaaaaaaagaacaaaacaaaaataagatttatctttaaaaaaatagctgaagtggaaaaggtttcgagatttctgcagccacgttctaaataagaattgcagaatactgtaaattcagatttacaaaaagaacacttggtggagagtggggcagaatttctgccgcattctctaagcgcttccaagagataaaatcctgtagcggaagatgcaaacgcaagggtgcaggggtgactgttttgagaactgctagagtgctactgaaattaagtggaggtcaagtcgaatctgattttcagacaattttacagtaaggcagcggctcactaaacaggccagttgacaagctgtagtcactttctgagtatttctgtaaaaatggtaagggatcaactctgcaatttgtccctcccatgaaagcacagtcttgtttacacctcgctggagaaataacactcgccctcacttctcccaaaaagctgaacccttcagtcggcccaagcagctccacaccctgaggtttccaagaccaaagctgcgagtctcagcagggaacagccacgtggcctgcctgcgcctcgcctgggctcttgccttcagcttgagatatctgcagccgcgaaccttgctccagcccagaaaggggcgctttgctcaattaattgttcccgccggcgagtccgtactgagaagcccatgagcggaccttatgtgcagggtactccagcgcggtgcacaaaactcgtcgcccccaaacgctgcccccaccccaacactgtgtactgactccagctttttactttgccatgtaagggatggacctgaaacggttattttacctcaattcatttcaaaaaggaaacaagtatggcattgcaaaagatgggcttcttatccaaggcgacttcctttctggttcaccaactttgctgcttccagtttgccaggatctacattaacaccctctttggggctcttcgttttaacttacagacagaaatgcttaaaatgttagcgtatccaagcatttggaattggggctcacgaagcctaattgtccactggatgccctagatagtgggggctggggcggggggggtctcagagcgggcagcccctatgtctaggcgctatcaaattcccacttcactctcttacaagctggcctttcaaggtcacaatgcggagcctaatttgggggtggggatgaaatggccacagggtctctcccttgggttggcattgccagctgttagggccgcagcaaaggcgctgcgctgcccccctctggctctgctgcctttcccatggactgggtttccttccaccgaagagtgaacttctgcctctttcgagcaccttccgaggcgtagtcctttggatgttggggagcgtcagactgggtcgttgtagaggggaaaggagggcccagaagggcgagagagcaggccgggacgcaaatcctcagcccccgcggcgcggccacgtcttcagaaacgcccaggacctccgggctgggccgccgcggtttggcctttggaactccaaggggttcgtctacctgaccattgggtgggctccgcggttgacacttttcttggcatgcccccccaccccgcgccacaccacccccccagccccagcaatcccaaatcggccccacggacctagagggctcttgggcgagatgagacatcacccactgtgtagaagctgttgccattgctgctgtcacagccactccggatggggctgccaccgcggccaggacagtctcctccgaccgcttcctgggctgcgctagggttcgggggcgctgcccgcacgctccggcggggaaggaaatcgccccgcgcccgccggaggaaggcgacggggagggaagggggagggcggctaggaggcgggtggaggggccggccgcccgggccaggtcgtttttgaatggtttgggaggacgaattgttagaccccgaggaagggaggtgggacgggggagggggactggaaagcggaaactttcctataaaacttcgaaaagtccctcctcctcacgtcaggccaatgacactgctgcccccaaactttccgcctgcacggaggtataagagcctccaagtctgcagctctcgcccaactcccagacacctcgcgggctctgcagcaccggcaccgtttccaggaggcctggcggggtgtgcgtccagccgttgggcgctttctttttggacctcggggccatccacaccgtcccctccccctcccgcctccctccccgcctcccccgcgcgccctccccgcggaggtccctcccgtccgtcctcctgctctctcctccgcgggccgcatcgcccgggccggcgccgcgcgcgggggaagctggcgggctgaggcgccccgctcttctcctctgccccgggcccgcgaggccacgcgtcgccgctcgagagatgatgcaggacgtgtccagctcgccagtctcgccggccgacgacagcctgagcaacagcgaggaagagccagaccggcagcagccgccgagcggcaagcgcgggggacgcaagcggcgcagcagcaggcgcagcgcgggcggcggcgcggggcccggcggagccgcgggtgggggcgtcggaggcggcgacgagccgggcagcccggcccagggcaagcgcggcaagaagtctgcgggctgtggcggcggcggcggcgcgggcggcggcggcggcagcagcagcggcggcgggagtccgcagtcttacgaggagctgcagacgcagcgggtcatggccaacgtgcgggagcgccagcgcacccagtcgctgaacgaggcgttcgccgcgctgcggaagatcatccccacgctgccctcggacaagctgagcaagattcagaccctcaagctggcggccaggtacatcgacttcctctaccaggtcctccagagcgacgagctggactccaagatggcaagctgcagctatgtggctcacgagcggctcagctacgccttctcggtctggaggatggagggggcctggtccatgtccgcgtcccactagcaggcggagccccccaccccctcagcagggccggagacct

SEQ ID NO: 21 - DNA, 6000 bp, Artificial sequence
  NPTX2
aactattgttagctatactcaccctacagtgctgtagaacaccagaacctagtcttcctatctagctgccatatgacattttaacctttctcaaactgtttcctagtcttcaaaattctataaaatatcttcaaactctcactttcaaagattcatacaagctacctgtgtgcctgaatcatacagagatattgagccacatgtctttatcagaaataggccaatttcaccacttgtaagttatgtgaaagtggggcactttcttggtgtttcctaatatagagaaattgagactacattcatagaagattttctcaaagcaaactaagttgatgatgttccttccttcagcctcctcccactcccagtctattatcccttacttttaaaaactgaggtaagacattatccctggtatatccggtgactcgttctattgcttgaaatcagaggatgccaccaaaagccttttttggtttgttgaaatcagttgagtaatatgaacaaccaattcccctaaatcacggctctctagctaaatgtgataatatacttgtacagggacaccacttcctgaaactcagtgtctcatctcacatgctcccaaaatttagccagaaatatgttattcaagaagaacacagatgcactgaagcaagaacattttgctttatctgaggggcttatatctatttgggtcccgtagcaacacaaattcagaatcactgagatgttaaatattacaaattttttagttgggtccaggcacatagtctggtgatatatcccaagtgtaggtgtgaaacagttcactttgactgaatcctctgattaaagctcaataataccatgctttatcttttttttaaaaaaaaaaaaacccacaaagtagtatatctttatcttgctgagacaccagtatattttcctgaagttttcatcccttgtctttcctcttgggaggctggcctgttctttcttaagtattcttgccttatttaactctaaagaatattgcagaaagggaagaatttttttcaactttgtttttaaagggacttacataatacaggtctaaattcagtggagaaaaggggaagaaaaggacaattatcatctattgtgcactggctatatgccaaacatgttcacttacattatctcactcaagcctaacaatattttgtagtaaatttagctttgattttagagatgagaaaactgaggctgggagaggttaaatcattctctcaaggtaaagtggcaagtcataaaactgggactgaaactaaagctgtctgactccaaagctcaagctctttccacactaccatgcagttcaatctagtggggaaaaaaatccactcttgaaagcgtcttagcattttaagcataaatgtgtgttagtagggatcctctggagcttccaaattctattttatctgcactgaactttacacacacacacacacacacacacacacacacgtctatttcccccttccaattgaaattcagcaccgaggacagttcccgaatcatgtttgcaggaagctgtccttggattctgttagagctcacactctcattcttgacttcccaagtggcactgagaaagagagaaggaaatgtaagaaagtatgagatgggatcctctcacaccatggtggaaattagcacatttcccagaagaccaagataaaaatgcatgtgagaagaacaagagaaccaaacagatggggcaataaaatggaggcggaatgagctcatcaggattcccagcacacggtttaagtccatactgggctcacagctgacctgggaccagccgacatagtgaatgagtgggtggttactatgctgcagcatcagttacactaagagattaggtggagctgattcactgctgcagatctgcacgacttttcaagtggaggaaaatctgtgggccctggtctctgccactcacataaccatcctcatgggtagcatgagtcatacatgcaggatctttcaggtagacaaagttgttgtgctgtgagtaaacccagtatgagctttcgtgttgtcgaaagcaggttttaaccacaactacggcgtctccgtgtatgaaggtttacctgctgtactacaaaacgaccctgcctttccacattcacctttggctacaggtggttaataacacacacagaaaaaacaagtttctgaaacgttcctcacaccaacagtgattccttctttttcaaactatcatgctcttgagtacagccaaagcacctttagacagttgcgtctaatccccttatctttttaaaattattattattattttgagatggagtcacgctctgtcaccaggctggagtgcagtggcatgatcttggctcactgcaacctctgcctcctgggttcaagcgattctcctgcctcagcctcccaagtagctgggactacaggcgggcgccaccacacccagctaaattttgtatttttagtagagacggggtttcaccatgttggccaggatggtctcgatctcttgaccttgtgatctgcctgcctcgggctcccaaagtgctgggattacaggtgtgaaccaccgtgcccaacccccgcccctcccttatcttatatgcaagaaaactgaagtccagaggagaaatgacttgccccaaaccacttagctagtgacagagttagaattagcactagatccctcattcctaagccagcaggcttttcattgcaccaggaagataaaataaaactgtaaatagcatgtactctgttaactaagcctctaattatactgcctccaaagaaaataacatttcaaatgtctgggtctttccatttgagctttggcaatttcactgatcacttctcatactggaatctcttttaagacgtttaggagtaattatattgggatatatgctatttttactcttgtacactgctttctttgttcaggaggaaattagaattctggaaagatacttgattttgtttaattattaaaggaacaagcttctacttcaagtagttgcaaatatgaatgtatcagtctgtgtgtcaagaaaggatatatggaacaatacaggaacgataatactctattgtcacatccaattaagggccacctaggtctacaagtaaagaggaacatcaaagctatgagtgaaaatggaaaagtcacatcgttatctgaataattttgaaaacgtctggatttggtgccttatgaatcatctgaaatgtaacaaggcataaagtgctttgcaaccctgttgccttcattattgtaatttgtgcatatgtaggtttatttgaactttttcgagttttcatccagctgaaaaaggatgtaggaagaatgagtccagtcaaggtcatacttaacaaggtgaaactgacacctcctggggatgcaggctaacagaaacatgagcccattactcaccccaaatctcccatccatacctttttctgctaatgaatattcttacctgaaccatcattacttccattgtcctctgctttggataccttgcagaccaggttcctgtctctcatttttatccacgaagagattttcaagaatagaacttttcttataatttagcaataatgtccttgaaaaccccacaactatttacataatagaagctattgtttgaagtgcaaagcaggtcagaatttggtttctagagattaatcactgcagtctagtttatattatcataataatttcatttattttggtacaatttgctttcagaatcaacctcggcttctgcgcacaagtgacaaatccatttgtttgtggtataatggataacgtgattaatgaccttgcaacaggatttcttaaaaacatagagaggaaaagaaataaagatttccatttataaatgtgcagtaaaccagctcagtctgtagctgtgcaccaaaatccctctgaacttgttttaagccacagatttggagagattctgaaacaaatttgctcctgacttttgggggtttctgctcatatctgctgtctccaagaagctgagggcagtggcctcacttcgaggaagtggtgacactgcgggccctcctggttacaaggaccgggcactgttggaccacgtggctccatcatgatgactccagttagatgtcaccccgcccctgagctcaggtcttgctgaataaggtcaccgcccagggggcagtcgatgaacacgcgcgcgagggctctgcgagtggcctcgtgactttgtccctaactccgggtgtcccctccttcccatcagcgtccggcgcctggtcctggtcccggtccccgaggcccccgggattcttcccgagcgttttccgagttggcgcggggggtggaggcggggccatggagcgcgtcccggggaccgttgcatccggaggcggccgtcgtgcggctccttcccgcctcgagagtgaggtggccgggccttgacgagaaggcccacgcctgccgcgggggtggctcgcgatggcagtcggggttcgagtcccgcctggggggctgctcctgctggagaaaacgcctccctgagggcggcggcaaacgcgcagcgaggccccgtgccgcgccagaagccaccctgagaaaggggcaccgggacaccgaggggttcccactttctcctcagcctgtgacgcccgcgtcctcgggtgggttcgaggggcgcctgggcacggccagccgaggctctcgagagccccagtgtcgttttccacctcaggcctcctttcctgaggcagagcccgggacctcgcgctctcgcctcaggctccggcccacgctcccgcccggccgccaggcgcgcaacggaaagcgcccccgccccgccccgctccgcccactgcgtgacgcgcacccggccgagccaatcagagctcgtggcgcgcgccccacacgccggccccctccgcccctcagcttaagaaagggcgcgcggacccggcaggccagagtgccgagcagcgcggtgggtgcggctgtgagacggcaggagacttctgccccgcggtgcacgcgaccctcgagacgacagcgcggctactgccagcagcgaaggcgcctcccgcggagcgccccgacggcgcccgctcgcccatgccgagctgagcgcggcagcggcggcgggatgctggcgctgctggccgccagcgtggcgctcgccgtggccgctggggcccaggacagcccggcgcccggtagccgcttcgtgtgcacggcactgcccccagaggcggtgcacgccggctgcccgctgcccgcgatgcccatgcagggcggcgcgcagagtcccgaggaggagctgagggccgcggtgctgcagctgcgcgagaccgtcgtgcagcagaaggagacgctgggcgcgcagcgcgaggccatccgcgagctcacgggcaagctagcgcgctgcgaggggctggcgggcggcaaggcgcgcggcgcgggggccacgggcaaggacactatgggcgacctgccgcgggaccccggccacgtcgtggagcagctcagccgctcgctgcagaccctcaaggaccgcctggagagcctcgaggtagcggcccgcggggagcgcgggggacctggaatggggacgctcccgagtcgggggcggaagactcgggaggatggggaaagggggcctggccctggggagggtgtgatcgtccgtgggggtgagctggacttgagggtgaaaggcggggatctagatcctgctcgggaactcccctgcgtggtatcccttcccacaccgctgctcttgctggaaggaaacgtttaaattccacccccgcgcgtcgggactgccagcgggatccgccgagcacttcccgaggtccgggctagcgaacccagacggccaagccgcgggcgccaaatacccggggacgcggtagcctctatcctcttgcaaatctccaaatctccgcgagccgggatgcgctcccgca

SEQ ID NO: 22 - DNA, 6000 bp, Artificial sequence
  GATA4
ttactttataacttcagggggcatggacaagtgaataaagcacaagatcattttactagtccagggatgttaaccctggtcttgctctgggctcagcacaaacaacactgtgacttcaaaagtctctaactggggagtagcaaagccccatatgtgaaagaattggggcctgtgtcaccaagagagatctcagaggaggcaatccccgaagaaagggccccttacagactcaaagaacctccagtctgggaaccagactccctatgttctgggtggtgggtggcctgggcagtaggtgttagcagctctttcaggctgccattgtctccaggtccttggatggggagggagagagggagccaggttggcagcaggaaaagaaacatacatggcggcgcctgcgtggccactgcgcctccagcgctggcgctcctcaacctgctggggcccctgcctggacttgcaggcactggaccaggcttcagtcctagcctcagctacgctggacctgaagagcccctccctattcaaaaaggctatggtgtccgtcctgaactcagcaaaaatgttcagagattcctttccacttttttccctcttcctgtgggctctcagattatgagatataaacttttttaaacattgattttattttttaaatgttaaacatgctcattaaaggaaactcagaacaatttttaaaagacagtttttaaaaatacgttttcatggtagaaagtcgatattaaatagaatggaaaaaaaagaactaagattcaggaatccaggttctagacctgcagtttcgcccttgctttgccacctcagacaagtaccttaacctctctgagcctccatgtgctggtttctaaggtgagggcgataacaccgtctttcccttcctcttgctaatgatattgttgttccctagagaaaaatgagatttgaaagtgttctgtaatctggaagtgactataaaatatgagagggtgtgccagaaactctggtcctcagggctggaagcaccaggaagcatttgaggaggtctacgagggagaagatgtattcgctttgcaacccagagtagtaatttcgaaagcaagaccattaacaaggattgctccttcctctcctctcgtctgtaaccggctgcagagcacggttccgggcgaacagggcggaggctctacgtccactccgtatccccaagaaagagtgtccgaggcacggaccatagcaagtgaaggaaggtaggtcgacgtggccttgcagctgaattcgttctccatttttccttcagcagggacgcatcctgctctgcaccctggttctcggcgctgcgcccgcggaggctcgtgcagggcaggctgcccgtgcgggtgaggactgagtgccgcgcagggaaggagtatcgcagaccggcgcccaggcccagcgggggaatccaagggccgtgttgcaggactcggcattcgttctgcgcgggtcaccttgaatgtctgtccggatccctcgcggcagggccgcagaggcgcgtccatatcttggaggaattcgttccatagaatgaggtttgattctctctggggtttcttgttttccattataagactctggcgaccttggtggcgccagattttttcagatgttgcttttgttccgggtgtagcggccaagatcatggacccaggcgtggcacttggtttaaaacaaacttggacaggtcccaccaaaaactcgcagaaactcggcgctggaaaaccatcagtggcttcactgtgaattccagcatccaccgtttatttttatttttggggggaaccagtgtttagattgctctgtacacaatacgcagcgtacaatttgcctcttctggggtatggaccagctcaagtcccaagagccttaatggaacagggtaaagcaatttattcttgccttggagatattttttaaaagagtacagtacacctaagtaattcttgtttgtctaaaatctgacgacctgacactgggtcattagacccaggttcttagaaaaaaaaaaaaaaaaaaaagtcaaagacgttcacagtgttaaattctcctcctagactacgggaaaggaaacccgagagaggacttgaatggggattgggcctgtctacccaggccagcccaggcatatcttccttaaaaatagccaagaggccggacctcagagcacccacccgctgccccccttcccagcggcctctggagcgagagagaagcctgggaacctagagaggcgccgataaacctcctccagccggcggcccagcgaggccttgaaatgctccccgctcctggcaacgcacagccaacctgcaggctcccgcttggcccaagggagggagggggcgagcggagagcgaaggaggaaaggagggaaaaggaaacacccccaaaaaagcaggccgtttgccaaccacccctggtttgtccttgagctgaggccttggggagaaagttggggcgctggacctagaggaaaaagccacaagaaacataattttctctgtcccaggcgacttccagagacagcgaatattcctgggtcaggggatcccaggtttcagtccactaggagtgccagcggaaggtgtgggtaaaggaccggggtggtggggggtggtgggaggtggtaggggtggcccagggttggcagaaagcggcggctcaggtaatctggggttccttgcaagcaagcacccagcaaaagcaggcgttcccacccagcggtgtggcagcggccatccacagtacagcctgttatagccccaccatccacagcacagcctgttatagccccaccagtgtacagagcagggggttgaagtttagggaggatggggagggcgagggtgaaggatcgccgcaaggcaccgcacctcccgctgcagcccatcccgcactactaggagaagccggcgtaggagcgccgcctgtgtccttggctgtggggaggacgtcagatggcaccccgccagacactaagccccaagcccctggcttgttgctaagaaaattcactgcccggtccagactcagcccttttcgccctttaagggtcgcgcgtgggaggcagctctgagaccccgggtagcgctggagccacagatttcctccgagaaaagaaaggccgggatagcttcccgctcgcccaagcccagattttccactctccaggaaggccttgcaggtccctgccgcaggccttggcttcgcgcctctctcgctcgccccccacgaagatgattgccggtttcaaaccgggagcagggagtctgcttccttctccgctgagtccgaaggatcgcagattggagcgtgctccggagaccgcttttccgcagcgccggcctccgagatccccagcaccccttcagccttaagttcccacgtttcgggtccgtggcgccaattctgctaagtagcaggctaggaattgggggaagtcggagaagaaaccctaagtgtgtcgcccccagcttccgggatgcaggcccgccggggtctagaggggcggctgccgtgcgtccagcctgtgcgcaggcctttcgccgctcggcgccccaggcagcctcagtttcctttcctctgtttgcgccccagtgaacctccgcacctctcattcagggaagagaattccccgcgcagccgcgctcgtttcttcctctgggattttcctgagaatccccaggagttggccacgatcccatggggggtttccttctacccagccccgcgtcctggcctcgtccttaacccccgggttgccttcactcaggctgggaatccacgattgatttcctactacggaagcgggtggcgttcccagcctgctttcggagcagcacgggtttcgtgcagggtgttatcccgaccccttcccccatccctctaatctggcttgagaagcccgtgctggagagaaaaacgcggccttaaaaaaaaaaaaaagtttaaccgaaagcgtgagagccacccgccggctgttatctggggctgaaggctgcggtaatcgatgggttatttttacgcggtaatagggccctgtgattgctctattaacctttagacctgtctgagggactctccggctcgcagccccgctgcgctggggcctccaggctctgacgccgactcccaactcaggcctgacacattcccctcccccataccctggaagagccccctccatgaagaagctcccctggaccgcctggctccccagcccttgccacgtcccttggattggtgcagagccgccgcaggctgcagaaaaaagggggaaagattagaagagaggaggccacaggagatgggaagtgtcgccaggaagggatgcagattgcataaatacataaaattgaggctgaggcctgggctcccgaccatctccctgggattttgggaaggcaaaagggaggcttcggtctctacgctctgattttaggaggcagtctgggtgtctcctgaacctccaaggaatccggggctgggaggatccccactacccctgcccaggaactagcatccagccgggcaccccgggtgacccagtgccccacacaagatcgagagttgagcccaagaggtcaccttcttctctactggccccgcccctcgcccgccgctgcgggatgaggaccacaggaagggggggcggggagggagaaagggaactcattaataaagctgaccctgggcaccacagcgaacccaatcgacctccggctgggttgcgggtgattccccgctccctggcggtagcacttgggcattttccgcggagaccccagagcctggactttgcctgctgggggagctttccgcacagtcccgcagcctgcgcccagcggaggtgtagccggggccgcgcacccccgccccgcccttgcacgtgactcccacaggccagtcagcgccctagggccgagttgctgggccggggacccgagccgcgagctggggacttggaggcggccggcgcaggggccgcgagaggcttcgtcgccgctgcagctccgggggctcccaggggagcgtgcgcggaacctccaggcccagcaggtagggcttttttcttccctttctttgctccttcccgcggtcccccaaactcggagcttctccgcctttgcttgtctggaggtagagaggtagctagtgggaggaaaagagacgtgcgctactcacttcaccgaaattgcccaacccctgctctgcttttgactttgccttagcaacttctttaagtcaaagtaagacttgggggcaaaacagagaaatattggaagcgcctttggattctttccgtgtgaacttgaacgctttcaatccctgtccccgtgtgcacattctccaacccttgtttgcatatcgcaggccggggcctgggtggtgatggtggccgcgtgaagttaccgggactgacgggcccgggacaggctgcacggcagctcgcacatggagggaagtagacggaggcttgtcgcccaccagcgactccggggacgcagggtggcagtgccaggcagctccgctgggcctcaggggcccccgggagccgctctgaggtgcggagaggctgctgagtggcggaactattcatgccctttctggccggcctcctcgccctcggggctggggtccagggactgaatgctcctctggaagctcaccaccccacctgcccgcgctgcttctacctgaaactggccaagggcccgagcccggaccggagccgtgacttccctccgccggccacggggctgcccggatccgccgggttatgtcgcttggctttgggctcaggggtcaccgtgggcagaggggggtgccggggtcgcggactgccaccaggttgaggaaaggaggggccttttggctggggaaagagcgtggtgggggacccgcggccgatggaatccctggggcagcgcggcccgcaccgtggaggttggggaagcgcct

SEQ ID NO: 23 - DNA, 6000 bp, Artificial sequence
  ADRA1A
ttcgataaaggattagaacacaatgcttctttggagagctgtgacttgatactgcatcaatacctttctgagaattgtttttcattttcttgcctctttaacttattaagccttaggagaattagttgaaaagccaagtctttggggtagatactaacattaagtcttctactctgtcatttgcaatcataaattccagaacacagctcctaattccattgtgtattgttttctaagggaatgatagacagattctttatttttttaaacctctaagcctaccacacttgccgagttcctcactagtcactaagaaagtcctgccaatcaatgcatgggtttatgtccattgctcagctcttctccaatcagactcattcccccagcatccctgacacaccactctaaaatgcggctgctgatggttcaccttcctcacttttgtctacaaatctcaatcctgctgattccacaaatcctacatcaagcaatatcattttatgagtctttccacaaccaccccttcaggggattcttcaatttctgtcacaccggaagtcttcagagtatcaccctcagagccaggcaagagggaccccggctagggtttcaggctttagagagtccagctctgactccttttggccataggactaatgtgatatgcccacctggagcctgtgccctcctttctagaccatgccctgggactcagaatcccttgccccagatggccacacaatcactttcaggtccattctctctgggcagacaacatcacaaatgtgtgtaccccaaggcctgaggccaagaaggcagctttctggctgtaggggctgaggtgttcacacacatttgcatggcccctcaagacaaagaacaagggggaaagtgagaagaaaagaagcagccagtgatcagggccagctcttgcaacttaaccatgttgggtcattctgattaaaccacttagctcaagtgtagtgctcaagacacttagcacattctccagctgaatttaccagtgttcatggactacctgggttagaaatatatttcactataaagtagcatacaaaatgagcagaaagggagttaataagattaataatagagttagtgaatattatgagctgagtttttgagaaacgtaatttctttcaacactaataacaaccttgtgggggttcattgtctccctttaaaaattaggaaaccaaggctttgccatggtcgcataggagggtcagaatagcatctttatgaccccagagcatactcctctccactccacctacccatgtgtacaactcagacactttctgggatgtccacgtcaactattctttaaagagtaaccaacagatggatagttttctgtttgtgaatcaatggtaggtgactgaaaaattggttctgagaggtcgttttgcaaggattgatggtcacaggctgagaagcagatttgaaagacctacctgctagcagcatgaagagctgctcttccttatcttagtattaactagttaattattggaggtgggtgcaggggtggattatgtgtattcttaattgttgtagagtggggactgggagttacaaagacttttgcaattttcgaccttgcagagctgagcaattttcagttgctttgcttgctgatagcactgcttcccttatctaccatggaacacatcttaatgaagaatttgcattcacagcatcaggttaatgaatacaaaacaaaacagtgtatatccctctgatggatgggatttcggaagcacagacattatacacatatttgatgataaagtactagaagtgcagggaattgaggtcaagcttcctcctaaggggactgaatcccagagagagcaggtgacttagtaatgagaagtggagctgtctgttcaaccaggatgctcctcctatggcacgaaattcagttttaaaaatatattaaattcaaatcaaatgtgttaggtgtgagttctatccctacaggtatgaggcagaggtggaggactttgtatacaatagagaaataaatacatatattaggtcttccatgacataggatttactgaccctctcatgggcattcctctgaggcattttgagatttattgctataaaagagcctcccaaacattatctcacttagaaaaggtaatcatattaatatgattttgttcacaggagagaatttaagtgccactgcttaaagttatctccttgttcctaggtttaaggagacctagtaaataagaacattccactttgtctgcatcaataaagatgaaagatgacttaggaggtgggaattggagtgggaaacatttttctatgttcccgatattctgaaacacatgtgactttattcaatcacaaggtaaacagattatgtaatttaccagaaaaaaagtaataagactggtggtgctaggttttcatactccagctattaatgaattaaagagagtaacactcctgaaaggataccattttctcaagaaaactggaaaagattgtgtggcatttaaaaaataccaaactctgtggccataatgctcttaaaattcatctgtctaaagaaattagaagtgaatcatattaaataaggtttagatatgtccactttatcttcctgaaaatataatttcattacaatcagatttgtcatattttatctgattttacttgctatttaaaacaccttataatttacttgcatatttagaattacaatattcttaatatacttcttgatcttaacaaaacctaggccaaatgttaatcaaatcaagctgttcaaagttactttatagcacattcctatgaacacaccatacacacagcaatatctagcaagggtgtcaatttttcgttatttttaaaagctcatttaaagaagttatttactacaaatgactctacacacacacacgcgcgcgcgcgcgcgcacacacacacacacacacacacaaacctttttaaagaaacgctagaacccaaccccctctaggccagaggaaaacattacagctgtatacgcacttgtgcctgttgccgtagagtaatacggtagcagcaggagattacggtactagctgggctactgcctgagttacgtcagcgagagctgcaaagttccttgctattcttttctggtgtcggggagctgaatattaaaagggtgattgtggagttaccggttatctgcatttttttttcttttcttattttgactctttttaaaaaatgcaggtaaagtgacagcggttcaggagcttaaagacatcagtggtggaggggtgagtcagcgggtgcaaaaggacaaggatttggtgcctcggagacacggtcccctctccgcctccagagaagagcaggcaggcagctcccgggaccgaagccgggtccacatcccccgcgcgcgagctggtggctcagcagcggcgcttcaggtgagtgcgccggggccggcgtcccgcagggccgagtgggtgagggcagacctcccccgccgtctggtgagacggaacccccacttttcccagcgcctcccgctttttccaccaggttttataccggcccctctaccccacccccgattcccttacatcttctgcgaagttgccttctactgaacaagtgtctttttaaccctgtgtttatcaccctcgaggtaggaggaaaagggtttctgcagtggcacgtttttaataccacctgtgaggtctccaacttgcgattttaacaagagtctttgcccgaggtcccacctcagggcccaaccccagaaggcaaggtgggcacttcctcacgccgcgctgtcctgccgagtccctgcggtaggttcgcagttgtggaaacccaggtttcttacgcagatggtggcccccagcccagaaaatcgaaggcggcccctgcccgctggcatgccggcttaatgtttacgcctgcaaaatccgcagtgactgtcacttgcaaagctccctctgcagagggacgtcctccccaccccgtcccccgccagtcccgctacggctggcagctggagcccctcgggtggccaacagtgaggcttggaaaggcgtcgtggacagacctgggtcgctttctgtcttcgggtccctcccggcttcgctcgggacctggctctcaagccagcttggctggtggacagaccggtgcgctctgcacacccgagtgcgaattccaccggcgtgagagtgagcgtgctcgtggtcctggccctgaggtccctgggtcgcagctgttccctctcccaggccgccccctccaggtgactgcgaggcaacctgttctaacggaaaccgagtacatcctccagaattccccggctaggatccgtgcgacacactcgccagccgcagtcgcccctccggggcttcgaggattttaatttcgtggtacctgcgctcgaaatccagacttcgagcgctggagcctggggttttggggatttgtttttttgtttgtttttcgcttcggatcctgaactcgggcagaggtgactcagtagagtgcgctaggcaggttcccagtggtgggggcgcgagatgagctccgaagtcgcctccaccgctgccgggcgaagcagcttctggaccgcagaaccaacccggctcccaactggtgtcccccaacccgtcaagctcagcacagcctctttccctggggcgcctagctcaaagccgcctttctctttgcgctctttcaggtggacgcggtcaaacgatgccccgcagcctcctgggtctcagcacatattccacacctacgtcccctgacctgtgctcctagaagctggagagagcaggagccttcggtggggcagctcaaaatgtaggtaactgcgggccaggagcagcgcccaacctgtagcgctgcgctacccaaccatcggtccctgcctttgagcgtcgacggctgatcttttggtttgagggagagactggcgctggagttttgaattccgaatcatgtgcagaatgctgaatcttcccccagccaggacgaataagacagcgcggaaaagcagattctcgtaattctggaattgcatgttgcaaggagtctcctggatcttcgcacccagcttcgggtagggagggagtccgggtcccgggctaggccagcccggcaggtggagagggtccccggcagccccgcgcgcccctggccatgtctttaatgccctgccccttcatgtggccttctgagggttcccagggctggccagggttgtttcccacccgcgcgcgcgctctcacccccagccaaacccacctggcagggctccctccagccgagaccttttgattcccggctcccgcgctcccgcctccgcgccagcccgggaggtggccctggacagccggacctcgcccggccccggctgggaccatggtgtttctctcgggaaatgcttccgacagctccaactgcacccaaccgccggcaccggtgaacatttccaaggccattctgctcggggtgatcttggggggcctcattcttttcggggtgctgggtaacatcctagtgatcctctccgtagcctgtcaccgacacctgcactcagtcacgcactactacatcgtcaacctggcggtggccgacctcctgctcacctccacggtgctgcccttctccgccatcttcgaggtcctaggctactgggccttcggcagggtcttctgcaacatctgggcggcagtggatgtgctgtgctgcaccgcgtccatcatgggcctctgcatcatctccatcgaccgctacatcggcgtgagctacccgctgcgctacccaaccatcgtcacccagaggaggggtctcatggctctgctctgcgtctgggcactctccctggtcatatccattggacccctgttcggctggaggcagccggcccccgaggacgagaccatctgccagatcaacgaggagccgggctacgtgctcttctca

SEQ ID NO: 24 - DNA, 6000 bp, Artificial sequence
  TNNI1
cagaaatctggccctggaaccacgatgggcttaacagggggtgggcagagaaggcggggaggggtgtggggttggctgtcactgaagcgaatgcccctgagtagtagcgggagggccggtgtcgggaggggccgccgggaagacagatggtctgggctttgtcacttgctaatttgggcttctgtgcttcccaaaccaagccagggcagccagggctgacaggtgtcagcctctgaggtgataggccctgactccacaacccacggccttacaaggcttagctcctctggccagacacttccttcccttgccccgtggcctccccagccccgggagggacaaggatgacactgggagtcagtggcactagaggctggaaacccctccaggccttcccctctcaccgatgcccatctcaccatccctctttcctggacttgccttttcctcccgcgatctggccagctctggttctcactccttctcctggcaattcttccatccatttccattgagctgggcagtcacagaagatctgagagggtactgaccacagaggccattctcctgaggcctggattctggtcaaggctgcctcagcctctacctggactttgaaagaggataaagggggccagacatggtggctcatgcctgtaatcccagcactttgggaggccaaggcaggaggattgcttgtgcccaggtgttcaagaccaacctgggcaatataaggagatgccacctctataaaaaattaaaaaaatatttttaaaaagaggttaagggaaagccagcagccttgtcccagggagggggaccccatggaagccaggctcagcctcaggtccctgcacacccttaacccgctttacaaatgaggaagccaaggttcagagaagatgctgcatagctggatcaattctgcagtgaacctaaattcagcttagtgtctagaaggcctgcaataaggctaggccaatgcaatgaggggaaactcatgtggtatagagtagggcttggacatgaagctggattaggaattagaacaaggtcagttttggcattttgcatgctgtgtgcagaatggattgagagcacaagcggccacctcagtaaaccaagcaagagagtagagagcaagaaggtgaaggttctagtaggttagggataaagacaggggcagggattatactggggttaaaaggagggttagggccaggtgtggtggctcacgcctgtaatcccagcactttgggaggccgaggcgggtggatcacgaggtcaggagatcaagaccatcctggctaacacggcgaaaccctgtctctactaaaaatacaaaaaagtagccgggcgtggtggcaggtgcctgtagtcccagctattcgggaggctgaggcaggagaatggcgtgaacctgggagacagagcttgcagtgagctgagattgcaccattgcactccagcctgggtgacacagcgagactccatctcaaaaaaaaaaaaaaaaaaaaaaaggagggttagttttaggatttgaattgggctaaagttaaggttagggaagtgtctttgctttgtgggcagcgtgacagagcactgggctgggagttaggagacctaacccctcaccctgtggcctctcctcgaggggcctcgatttcctcacgggcacatggattggactggacggtcttcctgctctggcgtcctgcaatgctctcggcaagatcagagctggctttctggaaggccaggccctggtggggctggattgtggcccactcttcctcagggctgccttgctgtgaaacctgggctggtttgtttcactgaccctcccaggtcactgttttcctgactggtcctaagcacagccggaaccatgaagtccagggcccatcaaggcaccgaagaatgtattagtttctcattgctgctgtggcaagtcagcacaaataacccaagtttattatcttacagttctggagggcagaagtctgaagtcagtctccctgggctgcaaggcaaggtggtgggaggcctgcttccccctgcaggctctaggggagaagccgttccctggccttttttggcttctagaggctgcctgcattccttggctcatggcctccctccatgatcaaggccagcagcatagcatcttttctgacatctgcttccttcatcgcatttccttctctcagccttgctcctcaagcctcactcagctgagaaggacacttgtgtttagatggggcccaccaagataatccaggataatctccccatctcaagtgtcttaatgtaatcacatctgcaaaatccctttgccatataaggtaacagattcacatttgcattagggcacgggcacctgtggggaccattattcagcctagcgcaaagggtgctgcctggggatctccagggccaggagcactctctctggctctgtgtctaggaagggtcctccctgaccagtaagcatctgagtcagacagaacctcagctctgggacagctgtgcctgctctgcagggagaataggaagcctggccccagggcagatgtgcactgagaaggggtgaccttttctgtaggcaaggaggggaggagaaaggctgcagaggcgcacttggctggggcagtgagtgggccacagggctaagacctccagtggcggcccctcagctgggtgtgggcggcctgaatcattgaggcctgctctgcacccactccatacatgaaggaagatgggggtcaggggcaaggacaacagggcactgcattcttgcttttgaaggccacctttgagagactgcaggagagagacgctaaagaagtcagtgtgtgaactggggtcaaaggtcagggtggatctgagagggtgagctggaggctggattccacatggtgtgcagggatggtggggatggaattgggacactggggtgaggggtcctggtcccatgctgtccaacagagtgtgaagatgaatcactccgagtctgagattctccatggctcctgcccagggagggcccctggctgcactatgttctccacgtcttgctgcggacatccggccacctactttcttggcttttgttcaccctgctttgttcttctatttgtcgaactgtcttcttttgtttttccttttcaaaaagcaggataattcatgcagagaattcattttttgagaatatcaaggccttttaagaaagttattgtataggctggtcatggtagctcatgtctgtaatcccagcactttgggaggccgaggcaggtgcgttgctcaaggccaagagtttgagactagcctgaccaacatggtaaaaccccatctctactaaaaatataaaaaatagccaggcatggtggcgtgcctgtagtcccagctactcaggaggctgaggcaggagaatcacttgaacccgggaggtggaggttgcagtgagccactgtactccagcctgggtgacagagcccctgtactccagcctgggtgacagagtgagactctgtctccaaaagtatataaataaataaataaataaatggaaagttattgtataaattataataagccaaggcaataaactccagtaggctcatctgcaaagcccctaaattccttctcccctctagctgctccttttggctggagcctgccttcattatccatcacagcctctccacttggaatcctatgtccccaacccctatgcctccccagaccctgtcatttctccctggcagccgtctcacatagaggcttctcaaagttgacccgaccagaacagaattagagcaacctctattaggcagcagaatgtcgttgtagagagggcagggcctttacctctgtgggcactggggtgtgtctccttaggacacccctgcccattactctctccttctccaaatggggagcatggctggggctccctaacctcctgcttgcgaggcctctctctggcctctgagagggtcagtgtcctgccccaacccatgagatgacagactataatagccacaggattaacatagcaggcattgtctttctctgactatagggtgggtattatgtgttcatcaaccatcctaaaaatacccggtaaacaggtgcagcccctgtggctccagtcccctgggatctgttggcttctggctggagatgaagattagggcagaggagaggtgaattagtctcactgagttccaggcatgagactcgggtgtcctttggaacctgggaaatctagattccaggaaacccatctggagggggatgcagagtgtctgcagaccctcagacctccctgagcataaaggtgtgccctgctgccactgccattctgctcagccctggaaacactcactggggtcagcctgcaacactgctcacatcactccccacagccaggcaccctcgtatcccacgtgcaccagagccactgaaaaatccctgaaagctgagtctttagccctcttgggcttttggggcatgggttcaggggcctcatttccatattgccttataagagatgcagggttagcccaatggtcctcttcccccagctgctacttgcccctctgggccttcactgtggccctttgctctccctcattccccctcccagtcccatctctgctcagtccccacacctggtctggctgctcattctttccctttctgcagctcgagtctccccaggtgggtgctggtttcactcagttggtggcacctatgtgcacagaatttgcctctgctcctgagccacaaattcacatggctttccgcctatgtgcctgttgtgtgcattgcagcacgcatctgccctgtgaggtgtgtgtgtgtgtgtgtgtgtgtgtgtgtgtgtgtgtgtgtgtgaagggtgggagggaggaggggcagcaggagggggcagtgggtctgttctatttttaccagccagttgctgctggacacagttttcatagcctcccctcggctctgcccctcacagtctgcagtctacggcgaggcacaggccagcccagctccacgaggactgaacaaggtaagcgtctgcagcccagaacttcagatgtaggttgatcccaggcagagaacctggggcttttgtttaaggagaaagcgaggtcttggagctggagaaaaccatttcttgcctgtgcagttggtttgttggtagaacattcctaaaatactcccacagcattttgtcccacagaatggcatctctcctgcaaaactgttgtgagaggtgaaactttttcctggccatttggaggctgtgagcaaggagcagaatcttccgtgaggttccttaggagccccagggcagagcctaagggatgatgttttcacacaggatgcccagcagagtgtgggagggagagcgcacccaggggaaggggtggccctgggatcagagagccagggtgagtgctcaaagtcagcaatgcctgcagcggaagagaaggagggtgcctgacgcgcaggactcccagggagcctagaaggcagggccagagctccctgctggccaaggaaaccatcttgcttctgtgttctttgtctctgagcgacgctgttagctccctttgcctatccctctgtagtgtagcacaagagcagaggagggcagaccggcaacccctcttgactgctcctgctcagagctcctggctctgagaagcagcagtagatactgggggtcctcttggaggaaggcaatggtggcggcattttgggttggggcacaggagccagtgaacacataagccagggcagtatctacagggtaacccaaggcttggggcagggggccgtgtggcggtccctgactcaggtgggggctgaaggcagcacctgccaccaggtttgggagtcacagtcccacagcagctctgagattaggctttgctgttcagaactcaagatgggggcagcccagcgccatgcacttgggagtgttaaccg

SEQ ID NO: 25 - DNA, 6000 bp, Artificial sequence
  TBX20
gttttcttttgcagttcttgatcacgctgcgagttttgcacacaatgttagcacgctgattcttccactgccgggaggaggcccggggctctgagggccggtccctgcggtagtgagctcgtgggggatgaacggggagcgagtggccgcggggaggagcgagagggtcgcagccccgagggctgtcctgtgagcaggctgcggggcctggcgcgcggggacccggggaagacgctacaaggcctggaggcgccgcgccttgggacccggaggaggcccagggcggacacgtggggtggcctggggtcccccgcccacgtagcgcggctgctgaaactacatgtgccctgggctgcccgccgccctgagcctgaggccaggcgggtcaacgtcgcagcaccgcgggcttctgccagaaggacatttcccctcacctcctgaccacatcggccttggagatgggcgcgaagcgccttacgcccacaagacatccctttatcggctctatagctgggcgagactgaggtctcggacggaacgcgcgcactcacaccccatccaggaacttcagtgacactcaggggtgctggctctttgctttgcccttcgagcaagacttgggtaagggctcctcgagctttctggtgcggaggcttcctgggtcggcccgaggcctgtaggttcccggccacttcttgcagcttggctgcgagggagcgcttctccgggaccactttggctcgataaactaaaacccgcggggtgttcgtcggaaggatcctgggaggcaaggctcagacccgccctcccgactcgcggtgaaatccaaggccgcgccgcgcgctccccacggggcacggagccgcgcgctgctctcggcgcgccccgggaacgcgccgcagcggccttggaggggctgcagttcctcttctcactgttcttgggaagtctttccttttccgtaaacctaatttaggaatccaaaggcaccgaagcccgttaatgtcgcttttaaagtgtctaccagaaagagctggagggaggctgtggacaggaatctccggccatgttcgggagtagacagatgcccttgtgctgagaagcgggtctcccttctcccattctctcctgccgccaacggtggctgccaggctgtgccctccgcttggcaccctgccccgccgtctcggctctgctctcgaccacggcgcctgcggacggttggttagacctgccgggaccggtccctgcagctcccagctaagggagcattcgttagcattcgttaagcgagggaccattgtccaaaagccggaaaccgattacttcctggacagttttgggggtcgactgtcttctgagttccgggagctacaaatgagaatttgaaagagtggtgtatataaggagacatcttccagtcttccatataatcaaattccatataaacaaactcagatgtataacaaaattttacattcttaggcgccaaagtttaaacgggatgtaaataacaatccaataccctcctgtggccccaatgctctttgtacattccaaaactatctacattatatatgagcacccgagagacatgtttctccaatcgcgaatttgcagttcaaaattaaatatgattaaaattctgtattatgctttacttatgttgggtttattgtgagtgttaggctttttatacacattagggcattaatccaaagagctatcttccaggtaggaatgaactattggccctattctaacgcagatgaagaaactgaagctttgagaggtttagtaacctacacaaagtctccgggctaatgcgtgtgttaaagttaacattaagacttcagcagcttccgctgccagcagtcagcctgagaagggcctagagagatgtgttaacgtgcttgttttgttccaacccccgggtggttttacctgttaacctgcgctatcagtggcgttttgaagggacctcagaactccccactctccgctttgggaaggctttgaatcatccttcgacagcacctctctgcccagaaccggttccccgtttttatggcatcatattgatttgcaaaggcaataaatctaggaggagggacggtgtgcccccgataataatgttgcagcatatgttttactgccgaggtttcaacaccaacaagatgcatagcctagagatccccaaacgggtcacccggatttgtttaaagtggccttttcaatacctctcgtttgccgcctgcctcctaacgatcactcatcttttcttgtatagtattttacctgtagtaggatgcgcagtcggggtttcctgcacacaagagacacagagctgggctaaggccgcgtaggcacttggaaaggaggaggaaacagatttgtggcaattcagtttttcccttgcttcatcaaattttctgaaaatgttcctctcatgatattattcatattccagtcaaaatcattgaggtaattaagaaaagacacagttgttttcctctgtgtagaaaaacaatcaaaagggtttttttatgaagtataattttgcggcaagtttttgaaaactaaattcagctaacaaggtttgccacaccctgttgaagcatatatcaaggtctagactaaccataacattccccgaccttccaaaatccccacttcttgagggaaatctttctttctggacaaatcctaagcatttgatataggaaaggaatcagtgtgtgccttccactcaatacaaagaaatgtcacttacattgagaaggtgtgtctggggagagacaggttggggcgggggactttaataaccaaaaaaggggtctggattgggtctgggttgtccaaaaccagcaaaaaatttaagaatagaaacggatcctcagatgcatcctgcggtttaatctgaaagccctctgcttgccagccgtcgttaactctactctggctcttcagctaacagcccagccacagctgcagggccaggggctgatgagggtcggggcccggagtgtgagtcctgcccttccgtctgcttctagccgctgaggtggactggggggagatgcgcgctctgccacgtcgggccctgtttttctctctcaaacgcacatggagaccggctagaagacagcgtaaattccttccagcaccaaagtctatggttttacgaatttcctcaaacatttataaatcgacacttcgaacttcgatagtgatatgagaatgcattttttagtcaccttcatccagcaccttgttaaaaactattgacacacgatgaccaagacaggctgaaatcacgactcttccagatgtcatcattttgttcacacaaacgttttcgacagctattttaacttgccgttttccctcacaaatttatttcaggagtagggggtggtgcagtgaaaagttggagttttaaaaattattattattattttaattttaagacccacgaagaggcttctaatagccagaccgactggctggaaaagagaacaaatatttacatatacagcttgagtgtgtatgtcagcctgagtttacacggctccaagcgaagcggattaccctgcgaattcggagaatttgagttattcaggctgagcagggctaacaggacgccctccaacaaggccgtgggaagtcctcgtcacagccgcctttgtaaaaccagaggggtctgtgtccgcttagtccgggcgctaccataaggttcgcactctcccactacgcgtcgcgtggttaccgtagagctccgcgccctgaccttcgccttctcttcggcagccgtcccattttccagggtccctctagggaaaataggagccccaggctagagacgcactggtgaggagcagaagccacggttctgagagcagcatcctttgccaaatgcccgcagctctcgctaagcttatctttttcagggctgcatttgctcagccccactgtcaaagagatcaaatttgggaccatcgaatgagagtcccagccctgggatctgccccgagtatggacctggccagttggcgccggctcagaggcgccggattctactgagcgcttccctactttcttttggaactttgagcgcaggtaagaaaaagaatggaaaaagcgagaactcccgaggcttccactggtctggtcatagcttccccaactgggccatgcccggatctcgggcgttaggccgcggtgatgtgtcctctcccgacagcgcgcaccgccctcccgcccggggctgtgagaccaggtggggatgtccatggctgctgcgtcagcctagtggtggcacccttttccctgaacctgtgctggggtaccgaacagccggggcgacaggccacgcgggcgccgcaccctgggcgcgccctccgcgcccggcccgcggccccgcccccggcggcggaatcaggaagcggtgacgtgagacggcgctgactggctgcgggcctccgggatcgccgccgccagcaaattaaggcgcagggcagcgagcgccagggctcactgtccgtagttccgcgccgcgctccccacgccagcgtcctagcagccgcgctcggctggtggccacctcagcctgggacatcccggctgtccccagccccagagggaggaaggacgcggaggggatgctccaggaccccaggactttgtgcagttgatgctcgtttccgccttcgggctgtgcagactgtcgtcctgccgagcgccccggggcgtgcgcacccgccgtagtgctcggtgggccctctcctctccggctgccttcgaagtctctgcggctctggggctttgcggtggggaatagaggccagtgtgcagctctggagtcgttggagctgacacttctggagtccctggccccgctgtgactgctctcggaaactttgagctgtgtttcgggtctttgtctcccttggggaatctggacggcagttcggacgaccccgtccctggccaggaccgcgtgctggggaccatggagttcacggcgtcccccaagccccaactctcctctcgggccaacgccttctccattgccgcgctcatgtcgagcggcggctctaaggagaaggaggcgacggagaacacaatcaaacccctgggtaagttgggctacccggctgtccgccgaggactggggatgctgcgcatccgtctgtgcccctggctgcaggcggctcgcagcaacgtctgatgctcaagccatgagccatacatcgcgggtgggagatctttttcttttgggtccaagttttctttggaggcttcagtctgttgaatgctgtgaatgtgcatcttatatctgggcccgtcggtctcctaagttgtctagaatccatatatcagatcagacatatggtgtgtgtgtatgtttgttgggttaaaaacattcatggaaaaataataataaaaccccttttaaggctcaacaggatttactgtgcatgtaacacctgcttgtgcgtttgaaacggagacgctaaaggacttcataagaaaccagtttaattttggttttcctctgacttgaatagacaaaacagagcaagctctttgaaaccactgtaaagcagaataagccagttctccctacacgaaccattttattcactgaagctctagacttatcaccaagacatcattttataactgtcgtcattttcagttgaggttggcaaaaacactaaccagtaaccatttctcaaattcctctacttttagttatgttgtttgtttaacaacctttaccatttatagaagacactaggctaggagtaaagggagaacagggattatgttcttcttaacactagtgaattacatggaaaagaaccaccccaagataaatatttaagaatctctttacaactgagggaaaaagaagtgttctcatcattgcctcgtggatccccaggaaataattgttacacagcctgatcgtctggtatcactacaatgtgaaaa

SEQ ID NO: 26 - DNA, 6000 bp, Artificial sequence
  ATG4A
tcactgataataaaaatataaattacctctatgaaatatcattttcacataacaaaaggataatttagtgttggtgagggggtaataggcactcttacactgctgttgggagggtaaaatgatacaactcctctggaagcaatttggcaacatatagcaagtcttaaaatgtgtgtatttgttgacgcagcaattccacttctaggagtctataagaaggtaatcatcagaaatgtggccaaaagttttctgtacaaagttggtcaactattttagagacaaggtcttgctctgttccccaggctggagtgcagtggtgtgatcacagctcactgtaacctggagctcctgggttcaagcaatcctcccacctcaacctcccgagtagttagtactacaggtgcatgccaccatgcctagctaagtttttaattttttctagagacggggggtcttgctgtgttgcccaggctggtcttgaactcctggcctcaagtgatcctcccgccttggctttacaaagtgctgggattacagacatgagccattatgcctggctcaatgtaccattatttataatagtgaaaaactcataaaagtttaaatatacagtaatagaggaatgattaaattatggcacatctatacagaattttatatacccattaaaaaggttttaaacatatttaatatatgcttatactatgaggtgaaaagaacaagagctaaaactacatacacagtatgaccccaattttgctaaaaaatatgtatgtataaaatatatacatttgaatataaaagaccaggaggaaatacacaagaatgtgatcagtagttatttctgagtggcaagataataggcaatttttatctttatattttgtgcaatttaacatttttgccattaacacgtattgcatttataagcagaataaaaaaagaaatgatgtataagaattattgcaagaaggctaatgtcaggtagaatacaaatgactgctacttgtcacttacagaggagtgttaccctcagtgtcttggatgtttgtggatgctttgtagtacagaaggatatgaatcatcttcaagttacccttggctgctgcccggtgcattgctgtagcctcataatggtccttagcatctggattagccccgccttccagtaacatgacagcgatctggaaagatggagaagaaagaagaaaacaatgcagaaatctatctgtgtttgtatacacgtttagaactcaacagaagtagcctaggatggaaccatacctcatgcctgtttttcgaagctgcataatgtaagggagtacagccattttgattgacagcattcacttgagcaccttttcccagaagggcttttacaatctcatcccggccagcagaagccgcaatatgaagaggagaccaacctgcctataaaagaagtaggtagtagaaataccaggggaaaaaggcacaaggattagtgctaacatttaggaagggttagcaaggagctttacaaaggcaaaaaaatatttgcctacaaaaaaggttccattcttactcttcacaaatctaatcctaatataaccatatcattagaataaatctctttatgtaacaaaaaagttatttaggtcctcattattcaggccatgttcaccaaaaaacaaaaaatccaacataatagaaagagtttatggtcttttagggttcaccataacaagccctgatctgtatgtttatttttgtttttttatttttagagacaaggtctcagtctgtcacccaggatagagtacaatggctcactgcagcctcaaactactaggctggagcaatcctcctacctcagcctctcctgtatgtttaccttaaatcacagagtatgagtggcaaacataacattgtaaaataatgcaaccatgagtctcatgcttgatcacaagggccatgatgtaactttccagtcataaaggttacaattttctttataagcttaatgcacaggcttcagtaaacaccagtctggagtacaaagataactgggaatttatcaactcaactttatcaaagtactcacatcgtctttatcattcactggcactccaagttgcaacaaaaattcaacaatttctgtatgtccagctgagcatgcccagtgcaatgcagttctgctgtcctacagagaagcagtaatagaaacattcttgaaatagaaggcaaaaaagcaagaaaaaaaatactggtgctggcaaatctatagtttgaaaatacgaaatgcagagcactggatcctcaggtaggtaaccttaaactatattacttactggtataagacagatgaaggtttaagtaacatggggagaatgacattgatgaacatacgtaattctgggcagaatacgacaaggaaagtagtagagcaagaaactgatttcaatgctttgacagaaatacatctttggttgttttactattacattatttacgtacttttatgtcaggcactgtgctgggagttttacatgtatagtatcatttaattcctaccattctacacagtgcttattatctccatttttcagatgaggaaactaaaactcctcagggtaagcaacttgtggacggtcatatggatactaaatggtgaagttggggcacaactgtcagtgaagttctgactctgaaatctctactaccacactgacctcaagttagatgctgtagaaggaacacatgaatctaatgggggaagaaatgttcattaaactagttgaaattatctagaatttaaaatcataaataaattagaaatataatttagaaaacacaacacacagaaaacaagaaaattactttttataaaaaataataagctatggccgggtgcggtggttcacgcctataatcccagcactttgggaggctgaggtgggcagatcacccgaggtcaggagttctagaccagcctggcaacatggtgaaactccgtctctactaaaaatacaaaagttagccaggcgtggtggcaggcgcctgtaatcccagctacttgggaggctgaggcaggggaatcgcttgaacccgggaggcagaggttgcagtgaggtgatacggcaccactgcactccagcctgggcaacaagagcgaaactctgtttcaaaaaataaataaataaaaataaataaaaattaaaaaagctatagtagtcattatttgtcaaagggaatccctatttgcagtgactgctatacttcttagccccacagaatagaaaatggtattttaaactctattacaaaaaaatttcaaacacaaaaatagagggaatagaatagaaacccacaggcctactgcttagatttaacaacaaacatcttgccctatttgctttaactagttttttccttgctgagctttccaaaaacaaatcctaaatatcatgacttttcacccttctctttcagtatgtaaaaaataaaggtatttcttatataactatgagaccattattacacttactaaaattaacactaactcctttagaatgtctaatatccggtctgtattcagattgcccccaactgtctcccaaatgtctttttgcagttggtttgtttaaatcaggaaccagacgagatccataaattgcatttcttcatgttttttacgtctcttgatgctgaatagttcctttccctttttaagccactgacttgactgaagaatggttaaatttccaatggagtattaaatcttaatataaagtattacatttaaatttaaatccttaaatatcaccagaccataatgaagtaaaccaggtaaagataaggccattttcaggttgagtaataaaggaaagttccagaaaagagaaaacagtctcttgatttctaattctctcccaaaagacacaaaatggagatctgtaagtatgggacagtctcactagtgcttacgacactgtctgcgaaacacaaggccatctataaatatttgtgggcttactaaaggcaccatgaatcctagaaactgaggatgaaagcacgagagtcctgtgagcaggagggcagaaagaaacacttctaaacttctgtctacctctgcttagaatgtccactcattcccccatggaaatcccactcatgcttcaaagtctaactaaccttcccttctttgcaaaaacttttccaattaacgacagtcatattcagcatcagatcctcctattaacgtgttctgaaagcaccatgtaaacttctctgccgagtatttaccacaatttgtaatcggacatgtttttgtgcttattttattgtctgcatcttccactccaccgtatccccagctcctagcaaactgcctggcacggaaggggcgttcaatttcacatgcgttactgaatacagttactagtaaaggtaaataaatccatataaaaagccttccttttcactgattgggagtttctgagaaatgcggtagacgttctagaactatccaaaaatcagggagcggagctccgggccaacgggaaaatatctgaggtctgtggggctagctcccgcaggcctatcgcgagtcccgaggtgacccagggggactgcttggggcggcgggggcggggttctgcttctctcagaaacggggcctccgctagggccgggccccgaaagccacgtagggcttcgccgacgtcgccgactgcggagagacctagcgttgctttacctggtcagttctagtagccagggatttatcggccagaatactctccttcaactcttccagcttcccgctgtaggccaggttgcagaccattaggttagacacacacccctccatttcgctgtcccagcaactacttgtcgcgcgagcaacgcccgcctcacgtcgccggctccggctacgccagtcaaaacagccgttagagcttcaccaatcaccggccttcctcgttcccttttcttttcccgtcgcgcggcgcctctgggagttgcagtttgagagcagttccgggcagggaggcgcctttgctgccctcacagacttggcccctagcagtgcagaactacaagtcccagggatcctagcgaccgtccgtccgtagtcaagttgccggtggaattggcccaggatgacagctggagaatggagtcaggtacggggagcggctttgagtggaaccgtgtgaaagagccggggtgggtagtcgctggcgggtcgttgagtcggccatatgaaacaggttcgggggcagggcaagagttatgagagcctaaaggtcctgtcccccggggttcctgacctgcagtggcaggcggaagggacaagggttggagctgagtactacctactgagctcgaaccggtgactgtggctaccctccccctccctgccaccgcctagggagtggaaagaagtgggggttaattactctgacatctcggcggtgtggcatcaggacagggttctaccacagcagtgatagcatatggcaccgtactgaggtgatgtgccagggtgatgccaaaggcagggtgtgatgccgtgggagccacctgatatctaagagacctcggtgagtccatagcctggtgacatcacagcttgatgagggtcatcttaaggataccgggctgtggtgttggcattttaatatcacgatattaactatgttgtcaaggctttgctacaactattaagctaatagtgtttctttttatagaaatcccaaatagtgccatagcagaagcaatgcaagactgatcctttatagggcatatccagaggacattatgccacaactaggtaacgataagaatacattcaagaaagagactcatgatgtttatgtttatcctgttatagtgtagaacaggagttgggaaactttctgtaaagagccagatagtaaatattttaggctttgtaggccaaaaggcaaaatcaagaatattttgttggtacttttatgacaaaagagaaaacaa

SEQ ID NO: 27 - DNA, 5449 bp, Artificial sequence
  HIST1H2BN
gaatactgaatatggatttttcaagataatgtctgcctctcggtctcatttaaattaccaagacatactaggtgctgtggctcctcccactaatcccagcactgtgggaggtcgaggcaggtggatcccttgagctcaggagttcgagaccagcctggccaacatggcgaatccctgtctctacaaaatatacaaaaaattagccaggtggtgtcacatgcctgtaatcccagctacttgggaggctgaggcaggagaatcacttgaacctgggaggcggaggttgcagtgagccgagattgcaccattgcactccaacctgggcaacaagagtgaaactctgtctgaaaaaaaaaaaaattagccagttgtagtggtgcatgcctgtggtcgcagctactagggagcctgaggtaggaggatcacttgaatcccagaggtggaggttgcagtgagtggagactgtgccactgcactccagcctgggtgacagcctgggagacggattaagaccccatctcaaaataaataaataaattatgaagacaatcatttacaagctaatttctttctgtggcccatttattttccataacaagcctttattgcccctcaaaggaattgtctacctttcccatctcctccttcccctatgaaaaaagttacataagcttctgtactcctttagggactggggtaatcactttgtaattctccctcgtgcacattattaaatttctgtgccatttctcccattattctgtcttttgtcagttgattttctatgaaacttcccttagcccctacagtatttacctctttgaggtactgtaagaattaatggaggccagtctcagtagcctgcctgtagtcccagctactctggaggctgaggtggggggattgcatgtgttcaggagtgggaggattgtgcaagctcaggagtcagagatcagcctgggcaacatcacaagaccttcatctaaaaaataaaaaaaattaaaaaaataaaacaatggagataatgtatgtaaaatattaagcagaaagccaacctctatttaatatacgtaatttttttctttacaactaatttacaaatattttgtttatattatactttaaatatgttaatacatcaatttatctaattatgtataacacattaagtagttaatttaaataacaaatatttatttttagtacatattgtcaatgtcggggctcagaaaccaataccccaaactatggcatggtgacaagctgaactgcagaagcctcaaagtctctttgaccttctcccactccccaacctttgtcttcctgttatctggactcaccaaaaatgagtccctgtaagacgaatgtaatcacacccgaacagctcatttcacaagataaggtacaagtttaatttcttttccctgatccattcattcttcctagtaatcccctcaaatgaattcctcttctccctccctcaaactgtttttcaaggatggtatataaacttctgaaccacgttctggggtgggcaatcactgattctccccatgcacattgtaaatttgtctgcctttttttctattaatctacctcatgtctgatttttcaacaaaccttcagagggcatcaaatcagtaaggacattcatttaattcaatatttcacagatgatataataccatgaagtgataaggcactataaatgttcctaattgctccttgccccttggatctctctgatctttaggttgcctcctctagtttacttactgtgaagctactcatttaacagctcctccattttcctttacatgtgtatgtggaattataaacaatgatatatcacacttgtatattttattttgcttaatacataagtttgcatgtcaatgttttgattatgagtacattgatttgagtttgcataaataacacccaaaattataataattataaagcaaaagttactttacaagctgtttagcttaaagaattttacttctggaactcacgaactctgaaaaatattacttagctagctttcatgattaaatggtggttcttgggagaaacagaatcaatggaataattttattatcaacttcaatgatcataacagcatattttatttaaaaacttattttaatttcaaaaatcttacaggccttctatggagctgtatcatttactctatattgagaaacaattattgaaaatttatacacataaataacacagatgacattttaaaagggaacaaatgaattaattcattgaaatcaggatggagtcctcaacatcaaataataaatttttttaaacaatgtgatatttatgatttttgtgattgctgttttattccaagaaatactagttgtatgtgattgcaatcaagacttaaatctaggaaataaaaatgtgtgaagagactcaaaaattaatgtatttcaatggactggagatcctgtagaaattcattttcattaacatgtctgtatgaggaaaacagtagcttatataagtttaggtggagaggaatagaaggccctctctatagctaggaggacttggtgatctgagaaggaaaggagtgatcttggtgaaagaaaggaagcaggaaaaactgacaggaacaggagtatatgaccatgatataataaaaaacctgacttacaatgtccttcattcatactgtttttaaataacacgttctaaagtaatctcacaccctaaccccaaccctttcccatcatttatataaacagtccattatgacatgtattctttgcccaagccaacttgaagctccttttggatagagactttatgtatctggattaatattgtattcccatttaggagagtgcctggcacaccatagatgctcagaaagattcaattgaactcgaataggtaggccaaacacacacggggtcctaaattgtgcagaagggtcagatacctaaattttgcttccatggtcttatgaagtataaactaaaagcccaagtaaggagcaaaggttagtttaccaattttatctctggaacccaggtacactgaaaagtattatttagctagctttcagtcctctgtaatacagaaagtaaatatctgacactaggttcatttgaaacactcttgctctgtcacacaagctggactgcagtggtgcaatcatagctcattgcagcctcgaactcctgggctcaagtgatccttctggtctcagcctcccgagtagctaggactacaggctatgagacgccaggcacggttagttattttacttttgtaaagatcaggctggcttcgaactccaaggctctagcggtccttccgcctcggcttcccaaagcggttctgtttaccggatggtgccaaacagttccaggctcttggtgcccggtagaaattggacgacacacacacagatagcaaagcaaagcagcaaaagtttagtaaacagagtattacactctcgggggtgggagagagcggactgacctctgcgaggtgagatcagcgtcagcttgctgtagtttgagtcattttatgtgtgtgcgtgcgtgtgtgtatgttttctgttcccagtgctgcctaatctatagccagcatctgcccttttattgataggtttgttgcttactttgtcctctgtggcttgtgcctctatcttataatcttaaatatatgcatgatatgtagcccatatgcatgaaccttaagtagctgattatcatacgggcttttgttaaggatacttttcctctctaatacgcatgcccatctctgaagagctgcctcttaaactggtttgttccagatcttgccggccacgaggtccttgctcacattatctcttttgtttcggctgcaaaaggttcactgcttgttatctcgcttcttgttcacccgcccatctaccttacttctgccctttgcttttacttattctgccctctaactttcagctccctttgttattctcttgcctcacttttcttattgtttcagctgtattgcaggcgcgagctgccgcgcctaaatttcttgatgtaccgtaaacatttcaatgtctactttctatctcaaaacaatgtggtgtaaaagccgtttagttttgcttcatctccatacagcattccagtgccattgcaaaatgactcgactatcagataaaactgaacacagctctacttggtgaaaaagtaggtggctctgaaaagaacctttttggtttggaccgaggtatgagtaatgaactgctccagccccgctacttgcccttggccttgtggtggctctcagttttcttaggcagcagcacggcctggatattgggcaggacaccaccctgggcgatggtcactttaccaagcagcttgttgagctcctcgtcgttgcggatggccagctgcaagtggcgcgggatgatgcgggtcttcttgttgtcgcgggccgcgttgccagccagttccaggatctcggcggttaggtactccaacaccgccgccaggtacaccggcgctccggcaccgacccgctcagcgtagttgcccttgcggagcagtcggtgcactcggcccactgggaactgaagacccgcccttgaagaacgggtcttagccttggcgcgagctttgccgccctgcttgccacgtcccgacatgacgtaaaaaattcaatcagtaacgttcctgagactgacgtaacgctaaagctccgctacttatagtcaacagaggcacgaaaactaagctgtgctattggctaacattacagtttcgctttaaccaatgggattgcggttttgaaaaacacttattttgattggacaaagttaatatacgtttccaggactcaccactggttaaacgcacaacttcattctctaccccacttgcgttaagaagcagtgaataagcggtaggttgacagagctaccgtcttcctgtttttttcctccaattttccggcagttactcccagtcatgcccgagccctcaaagtccgctcctgccccgaagaaaggctccaagaaggcagtgacaaaggcccagaagaaggacggcaagaagcgcaagcgcagccgcaaggagagctactccgtgtacgtgtacaaggtgctgaagcaggtccaccccgacaccggtatctcgtccaaggccatgggcatcatgaactccttcgtcaatgacatcttcgagcgcatcgccggcgaggcttcccgcctggcgcattacaacaagcgctcgaccatcacctccagggagatccagacggccgtgcgcctgctgctgccaggggagctggccaagcacgcggtgtcggagggcaccaaggccgtcaccaagtacaccagttccaagtgagcccgcccaccgcggaacgttcggtcagtctcggcccacaccccaaaggctcttttcagagccactca

SEQ ID NO: 28 - DNA, 6000 bp, Artificial sequence
  THRB
tatattcatattaatgcatttaggtctactttattcttttacctgtatttatttaaggaaaagattatttatgctgtgatagagtttggcccggctgacaggtgtgttaagggagcaccaattactgcaacaaatcaacacttgcctttcagtgacttaacacaaaagaagttgattacttttctcggtaatagttcaacatgagtgctctagttctcctccacatggtggtaaaagactcttccagattgtggcttactagttcccgcccagggcctcagaatcttctacctgtagttggaaaacaaggtaagagaatgtgattagggagttttctagccaatgacataacatgttttctttagggctcttctgatcgtcccggtgcacagtagttccttaaatgaagggctagagagatgtctcatgactgccagaacatgctcaccttttccagcctcatgtgcctatccaataactccttgtgttgcatgcaggtagcatgatgagctggttaagagtataccctctgtcacccagtacccaggcctgagcttgagcagattatttgatcttcctaatttttaacatctggaaaatgaagtacataataatactgtttcacagggtcctctggcaaactttaaaagatgtatgaaatatttaggacagtacgtaaaacataatacatgtctaatacatgttagctatgttatgtactccttgctataaccagccatgccagcatgtctactgcatctccaacatgccctatgcttctaccacaataaaggatcaagttttttccccatggccagcttcatgggcatgtgatctggacagctgcatacaacccatgctgggaagggcttggtgcttgaggcctggcacttcatttaatgctctgttgtcaccgtaatgaaattcctaataattttatctttgaacttttgttttgtaaatgaaggctgatgggacaatggagcatgtgcatgaacagaggaaatacagataacatgcatagctgctgccattctttacttgttccactcacacatagcattcttgttgccccataagcacagaatccctgtgggcacacaatgtgtgggagttctgcaaaactcaaattgagtatgaggtaagcatgtcacatcaatgactaagtgaggacactgacaaccccaaaatggggtttgccacgctttccattccaaccaaagcctgatttgaatgcaaaaagaaggtaacaaccaaggaaactttctctgtcttttcttatgttaatacttctctgtaaccacttagtttgaaaatgatgacatagaaggaaagagaaagataaggcaacctatagttccttttcctttctggtctcctttattcaaaagtaagccaaaggcagagtgttggtaaaaacgggcatatatcaagatatgaaataaaaaagttagttttgtgctgtgaattagagtgtacttgaattagataattttgaccctgtcatcttccccaagaagctgtctttcctgaagggttggagttcttacccagctgctatggaccagaatcactggagccctcatagaaaaaataagttccaagaactaacaatgaacagacagaagcagaatctccagggctaggtcaggtggctgcacagccagcttgagaagtagctgaataagcaaaacaataccaggggttccagtcccaatactccctggcaaaatgtggtccatctttgatggattggttcaagtgccaccattttccatgaaatcttccctgatttttccctcccctcacgcccacgtctctaactccctgaccccaaatccttcactatatattaattatctttgacatctaggacattctaacttgaactttgcttctctgaaaagactctggtaacactgagggcatagcagggcagggactaggtttaggcaagtgaggcactcatccctggtgtgaaatttaaggggatgccaaaaaagaaaaaaaaaaaagcctcagtaaatcaggagaaatattttaatgcactattttaaaaatcaaaattaatgcaacaattattcatgatgaacaatacatcaaaattttaaataaaggcaggctcagaccctgcacttgtatcactggcctcactcacttctgcccaatccgagccctatgaggttctgtttcattcattttcttacaccagtagttgctaaataaataaatatatgaatgaattgctttcttgaactgagtcatcagcttaaaggtatagatcctggtaatgaggatgtctatggtgaaatatgggaatgggtcctggaaacagccttggtgtgccctctcaagtcagctggaacacatgaccacataatctaaagtttgaataaatgttcttccttcaatcaagtcatcatgacattctcctccattgtcctaccttgtgtaaaaccagaaaaataaatgacatgatctcttggtaacccttctttaccatcatcatgcagcctgtaggacagccctcagaggtcctgttcaaaatggaaccctagtagttcacgttatcattaacattgcagtaaaactgccctcccccattgttgtagaacattgttactaccacagtctcatggattgtttgaataatgccacgccctttatttatttcatgctttaattggctcatttctgaattttctgggcaaaatgagatcagaaagacaaagcccttgaacaaagagaagcaacagtttttgttacatttcattttgtcctctgtatgtctccttcttaaataatttagctccaagttatactcaggaagagaaaaaaattaaaagttccagtaggccgagaagcccatgatccaccgtgttgaaggaagatttgcttcacctcaccaccccccaaccccctcccgcccccccgcggtaatactaagctgttcacacgctgtgaagaagacccgaagactaggttgtcaactgttggggcctacctgcgccagacttcttcctcagtggccagctttctcaccccccgttagccaccagggggccgcctgcttggaacaagtggtgtagacgccacagcttttctccaaaccaaccaaaacattccggggcagctttggagcaaagcccaggaacttccctgcaaaggagaacagctctccaactacagaagcctgcaagtcctatggtgcagacttattaagagtagagaagaaagcacggacttctcaccgaggaccaaagggaaatggggtctctggggcctgcaactttcttaaaagtgcatcagtgaaaccctgcttatccagcggggtggttgttaatgctgtcagcaggggtggggggcctctcacccctttccctgggacgctcagaagaagcgagaataaaacatacatgtcactggctggaaatctagagaaccttccaataaataacctatacattgtcaggcagctgaggatccatatcgtcataactctattatactgcgtgttacacgaatagatgtgaatattaaattatgatgtcggaattattttaatactgtctataagaaattaattgtactctgttgtcaaagtagttttgttgcaactatagttccttattgactaccttttagctgagtgaggactcggtatttcccaagtatccttctagctcagaaagcaagtctcttcctggtctccaggacttaaggtcgggggcatttgagagacctatatttgcccgggaaagatctcttgaagagtatacattatttttgtcttcctggttttatctatacatttccagggcaaagacaaattaatagggcggccgttctgggacctgaggagtgtctcagccctgtacgcgcctctcccacgatatgcataatggcggtgggggcgggggtgtcctccttaagggcaacccaggcacgtcccaaacttccattcgtggggtggccgtggtggttaagagagctgccagatggtcggaccagcggaggccccaagaagagccagagcgccctgtattcccgacacgcgccacaagtggtttaggagcggagggaggagcgctgcggcacgggtcgggccggtcgggccgggcaaagaaggccgagacgtgctcctggaagactcgccctccggtcccggtgtcactcgttccccattctttcctcttctccaactagagtaatgacgcccaaacccgcacctatgcgcgcaggcaatcgggtgggtggacgcgcggccaccaaacccacagcaccgtcaccaaccctgggagggcacggccgggattaggaggagggggagcgcccacacctggggaggcaggtgcggaggcggccggcccggggacctcgagtgcgtaggactcggggtcggggtcggggtcggcaagccgggcgctgtgagcgcgtcggagcgctggcccggagcctggcagggggcgtctgtaccggctgggcagcccggggcggtggcgatggctggcggcggcggcggggtgtgcgccaggaggccatttcctcgctgcgcccctggcggagccgggtttgcctgctcttggccgccgccgccaccgccgcgcaagtcggacagccgtgagggctggaggggaaaccaggtcaccggttcgcagacgcggcgcggagcaggcgccccgggcccggagtaagacagcgcccgggaagcgggccggggcgggccgggcacgcgggggacccggagaggcggggactctggtgccccagccgcagtagcttcctacgcctataaaagtggagagaccggggaggtgcggcgcggccctggctgcggccgcctctcttcgcccaaggagttgacattttgcaggactcgcgcgacgcccagtcgccggcgctccccgggaccccgccgccgggaggagggggcggaggagggtggagactgcggggcttggccaaggaaggcgcacatcctcgggcgggcggccgtgacgcggcggggattaactttgcatgaataatgtgagtgcgcttggaaaagagacctcctgctccgcgggctcggggcaagagcccgcaggctaccttccccgggcaggggcgctcaacccaaccggctccagggcactggtaatttggctagaggaccgcgcggaggcagcggggtgagaggaggagggggcgacagttccaactgtccacagggtgggcgggatggtgacggagcgtcgcaagaacccggaggggtgcgggcggctaagccgagcgcgcgcgggcgggcaggcgggtgagcgtgggtggtgggggtgtcatcagcctgattacctgcctccgcggggcttctgcgccccggatctgggaggaggtgccctctcgtgttcgggcaccgcgcggcggcaggctgggagctacggagtggacagtggtggaacagggtggccgggctctgttccaatcgcagcggctctgttcctcaaaccccaagcccagctgttgacatgttccttgtgaaatggagtttggcatcctcagcgccgaacccagtggaagtttccatgatgaggaagttgtgtgacatgggggttcggaaaaggttggcaggccaggggggagggttaaagggactgtgggtctcccatcccccctttttcgcctgccccggaactcccgggcttggaaaggagaattatcctggatgttggcgtgcgccgtggccggtgcctggcgactgggcttctctccgcctcctgggggcttggcggggatttcgctagcctcctgggtcgcgctccttcgctttgcttctcttggcgcagcatcttcctctgggtctcctcgagttggttgtgataccaattgtgccaactgtgtgacctccaccccctcggagaaggactcatttgggggaattttcattcttagtggattttgcccttcttagcggcagttgtcactttgggggaggatttcccaatgaccattgtgggattatta

SEQ ID NO: 29 - DNA, 6000 bp, Artificial sequence
  STC2
atcatattttttgttatccttagaaatatattgtttactcatgaatccttaaatctgaggaaattaatctaggttattatcatgtaggactcatagtctgagactttgcttatacacttcagtcagtttcgccattatcgagtcttccatagtgctgtctctctgcattcatcttctgatttgactaataactgtacaacattttcctctgtcacttttcttctctttacagtttgtgcagaaaaatgaaaaatatagctgaatttacaactatatttgataaaagtaagaagaagaagactacaaaaatggtatcctctgtcttcttttatactttctcgagaaatgatgtaatacttcaggcaatgtgatcaaaaacctgaaggatgatacaacagtgataatttgtttcattcttgcatcatccttctaggtgattccatgattctcccatttcttattggtgttttttttttttttaccataattggcaataattgatgagtaatgatgaaataatttctagggtgataaaatggaagatgtttaaagatataagaaaattaagatctctaagatcctatgatacatcatcaatttataaattatttcaatagtcatatggtaacttcaagatagaatgagttttattctatttttgttaaaaactagatgttatctttcatcttcagaagacttctgaaaacaaacaaaagtcatgaaataccaatccttacaaattgttagaatttctcaggaaaaaaaaaaacaccttcaaaatctaaaattgggtccgatggaccctgataacatactgaaagttaaatcacattaatagatttggtcgtttcctggagctttcaaaatgtggctccctagaaaactgccccacaaaaagattcttccttcagccacttgacatccaaccattctccaaccaaagttaaaacccacccagagagagctctatcatactattccagcaccacaatgcctgcctctgggcccaaccagtactctccatggaagccaggcagccccttgcctgggatacgttctcctggagtttgtctcaagatacaacaaggaagaaaaaattatgacccatgtaaaacagagaggccttctggaataccattcaggcttcagagaaaggcgttaacaaaactcagactacagtcagatgcccagtgtttactactttatattccttccagtaaatgggtttattgtgttcctactctcttgggggtttttttaatgcttttttttcttttgtatttccagtcaatccatgaatggtttgactattgatctgtgacagagaggaaatgcccaattcacaaaagcgttgtgttcccacacttccaaagttggttgtttggggaactcagaatatattttcttccaagaaacaatattatacatgataggtttacaggccagcaccacaaaatccttcttaactcattgtgttgttgggcactgcacttttatggtataaattagtagaatataatttggttatagtattaagaataaacaaacccaaatattgaagatataatctcctttatcctgaattatatttaatgaaataaccagtttaatgaaaaaaagataagagcaaagatagaggcttttttttttttgagacagggtcttgctctgtcacccaggctggagtacagtggtgcaatcatagctcactgcagcctcaacctcctggactcaagccattctcccagaacctcagtctcccgagtagctgcgattacaagcacataccaccacgtctggctaatgtttgtattttctgtagagacggggtcttgccatgttgcccaggttggtctcgaattcccgggctcaagcgatctacctacctcagcctcccaaagtgctgggagatggaggctttttagggagagacctctgcttgcaagataatttgggaggcctgatagcagcagtttgttatttttttctgtccggtttcaccttgataccttttttcattttccttcatacaggtatgatgcacaggagccaggattagggtaaatttgagactcacaaaggaaaacagcaacggataggggagcaagagggaaaataagtgtgaaagagatctctttgatggggggattaaggcatgttattgcgaggagattggctaaatctgcattaattgatttgctaagaaaaggaaaggaaactggggagaaggctggagacccaatatcagaactttccaggcaggcagagtcgtgaggactgtgtagccagacagttccctctctgaccataaggtggtgccatcagcccagacagtcggccccaagggggcggggcgtgcagtgggtggagctccccacgtgtaagccatgttctccactcaggttccggaactcagaaagcctggaccaggcagcacgtgtcaacttgcacatcactgactcagcacccacgaactatgcccatccgctaaagccccttcggctcacactcgagcaagaacacgtctgtcttgctgggcatcgagacgcacgtgaagattccaactaattccttcccactcctctgctctttctccacaatggcagctctgggcccctgagcatttcctaattcctaaagagaacctagtctaaagcgccctacagtctcatccttaaataccgtcagatcttccgtgaggagcacagatttgtcacaaggagcccagccttcacaacatgccctggccgacttccccagtttgatgtcccactttcacacctaactgggcaacccctattcctgatacaaatcccaccggatcctctccatgcatttagccctgctggtcctcccacctcccatgcccacttttttccttgcctctacttgtcactatcctcccccatccttcatggtctaacccaaatatcccctcccccaaaacctgccactcagtgttgaccgcatgtaactagcaaagtggatttagacaggaaaacatgggctccaggcgtcaaccacagaaggccctggatgacacaaggtaggtcatgtctgcctcttcctggctgtgtatgcgttaaattgaggaggagggcccatccagacaggtgctgctgaacctcaggacagcagctggtccatccaggagacctcccaggcctcagttcccatctagggagggccctaggtctggagggacaccaaaggcatgagtgtggtgtgtggtgcggtgtcatgttagctatataatagtaataatgattattattactgtcccctgccccaccctcatcagccacagtctgccaaacttggcctcagttataaagctaaaggaatcagccagagagagggggagtcaatcacaacaaagtcaccagtctgagcccccatcccacttctacccctgcggcctagactggccatttcagcaaactgcctagtgattctgcaagggaattttcacagccttggcgggtcggactcagatgggcattcttatctggatgctttggacttggctaagtggcccagagataaacccttaagaactagtgcctgtgcttacgccacctccaattctagctactatgtgttcttttggttctaaaggggcagttggctcaggtgaaaaccaggaatgtcccttggaaggcaggagcaaacagcctcaggacgaactttgaaactatgtaggtcctctgagggcagccccaggggccccatggtagcaaaaggtaggcctagccaaaggctgggcaaagcaggagcagggagagctccatccccctggccatcgctccaggtatcctgggaggcctctgcccagcacactaagtgtctgcatctggcaaaagagggcttgctcctgccccatggagctctctcctatttctgcttctcaaaaaaaaacaggtgaccagatgccttcttaatgtcaatattgctcatgttctctcctacaaaagaggccacatagaaagaaagtctacccagctgtacccataagcaggagtgggaccaaatggctacttgttctcaggaaattcagagccttaaagagttagttaggagaggccagaatgccgctgtgtcgtttgtttgctttgtttttaaggatctacaggaggtagttttccataaactaggaagctaagttaactatgcaaacacaagcagggtgggcaatgtgaactggccgttttaatgtatttgtacccgcacgtccttgcacaaaagatccaaggctgcgcggagtaattgctattagaatagtgggcatggtccctaccgccgcaggttcggaccttcaaagtgacaatttatggatgctccctggcgctccagcgcaggggaagcccactctgaagacgccctccccaccccctcctctccttccttccgactcaggagagctcgacacgccggatagctgcggccagccgtggccatcctggccccccgcctccgccttccccactcccgcgtgcagccgggacacgggaaaggaaagctttggaagtcaagcgccggccaaaagatgacctgcccgcgtgtctcctcgccccctcccccagccgtgtcacatggcggccccaaccaggcgggcagtgcgccccgccgcggagacccagggccgcgtcgcctgggcagtgggtgatgaaacttcccaggcgcattaccgcagagggcgcgggcggggcgcggggcgggggtgggggatcgagagctggtaccggggctcacctgttctccaggaggagggtggggacggggggaggggcgagtgcgcgccaacgccgggtgcgtgccctggggcgcttgggcgcggcgctcgtgtccccgccctcccccagccctgcgagccccccgagctgcgccgtggggtgacgggaccgagagcagttcctgtccccggccccggcgcgggggagacgtgagcgtgcacacgtacacacacagcaggggaagaggcgctccaagcggcgcccaactttctccttccctccacgggccgggtgagaaagtagccgggggctatcccgacccggcggttcttggggagggggccgaacaagaaaagggaggagatggagataacttccccggatttagcttttttgtctttgtttttgttctcaccacttccatcggatgactggagagtaaaagggaacccggagcggggtggcgagcagcgctttgagaaaatgcaggagtgtgtttggagacgcgtaaagttgcctttcaagctctggcctccgggcacgcgatgctccgcggcgggctgactcagggctgccttgggcctccctgccaccctcctggaaatgatgcaagtcctgactgtcacctggatccctgcagcccagcctggaatgcgtctggattaggggaaagacgagaaacgacactccaggtgttgcacggcccaccaaagcgggaagatagggcagttgctcagaccaaatactgtatctagtgcttctgctcctatcttcaatcgtggggttctttttaatgcaaagtgtcacaaggccaggaattcccatgtgtgctcagttggcccacagcatcattgtgcctaggaaactgcttcaatttatcaagtcctctgggctgggaatctcactgaattccaaacggcggaaagaggaaactttcccaacccgatgtgggtgtgacgcgagccaggggccccagggacactgtcccagagcacaccgtccccctttaacagcaactggagcttggattcgctcttatattgtacagtcctttcgaccattgccctggagcacccgcacacgcgcacgcatctccggccgcgctcacacacactcatacacacgcacgcaaacgcgtggccgccgccaggtcggcaactttgtccggcgctcccagcggcgctcggcttcctcctgtagtagttgagcgcaggccccgcctcccggccgtgttgtcaaaagggccggggtctcggattggtccagc

SEQ ID NO: 30 - DNA, 6000 bp, Artificial sequence
  ENG
acacctggctaatttttgtacttttagttgagacggggttttgccatgttggccagtctggtctcaaactcctgacctcatgtgatccacccaccttggcctcccaaagtgctgggattacaggcgtgagccaccacacccggtccttcattttttattttttagagacaaggtcttgctctgtcacccaggctggaacacagtgacgtgatcacagcttactgcagccttgaactcctgggctcaagcaatcctcctgcctcagcctcctgagtagccgggactgcaggcttttaccactaagcctggctcaaatctgcatttataagaagcttccagaagaaacaagaccacactttgagtagcaacagtctagggcatgacattttatggccgagactcgttggtgggtaacaaaaccaacagatgagcttgtgacgagtagtgaaagaaaatgcaacaggttgggggttactggagggcatcacagccgcaaatctatctacaggaccacaaaatgcgtttccattatttgagtctgagtcccagggtctgtgttgggacgtgacaccagggtcgcaaaggtttgcgaaaacctggcttgggagtgagggtaaacaaagaaggaaggggccagcaaccaagcctgggggaacaaggaggaaccagggaggagacagataaggtgaccagggaggtgggaggaaagccaggacagaatgttctggaagccaagggaaaaaggcctggatttcaattatcctctgcctcttcccagccctgtggccttgcaaacctgagcctcagtttcttccatcgtaaaatgctgacatgacagcctcagccactgaagtggctgcaaagtggcacttggcacagggccaggcaacctcatggatggtggtgcaattccaattcttgtcttgccctttgaactcctccagaccagcaggctgccctccccttgtgcagatgaagaaactgaggctcagaaagtggaaagatctggctgggtgcggtggctcacgcctgtaatcccagcactttgggagggtaaggcgggtggatcacttgaggtcaggagttcaagaccagcctgggcaacatggtgaaatcccgtctctactaaaagtacaaaaattagccgggtgtgatggtgcatgttcccagctactcgggaggctgaggcaggagaattgcttgaacccaggaagcggaagttgcagtgagccaagatcatgccactgcactccagcctgggtgacagagtgagactctgtctcaaacaaacaaagatcctgcctgatgccacctggccagtgtagggcagagcctgggcaccctgctccgccctgtgggtctggccctgctgttatcaatggcccctggctccaggccagtgctggagacagtcagccgcctggtgggtccctgggggccgcctgaaattccttcagtggccagtggccagggtggacgtgctctgtcttttcctgcagccctggccttcctggccagccaggaggaagaaagagcagaaagtgtgcatctgccctctgtggggtccaggcacaggggcctggccatcagccacgcgtctctcgggcgtgtggacagacggcacctgaacacatccgtatcagtcaaagccccggcaggaaacagatggcacgttccagtaggatcattagaggagagtttggtgaagggtctgtttacacaggtgtggtcggggtgtagggaagccccaagggacggtgcagaaccccagggctggcagcggcgtgggctgtgaccaccctcagcctgaagaggccaggggaggagctgagtccacagctggacagagctgggtggagggggtccccaacaggaccgtggccttcagtggagggaggccaccagtatgcagtgaccctgcagggaaggggctggaagacggaccctcctcctcctgcctcctctgacctctgcaggggtggggaagtggatgtgtcccacaagagggagagtgcagctgggggatgtagaagacagctaccccaaggcctgtgcccagcagggaggccatgtggccaccaggcttcctcgggcaggagacccagtccaggactggccttttctcctgggaaccaatgacaaggcccacttctctgggcctccatgacccccacccctgccttgacttctagggtccctcatgctttcagcaaatccatctcaagagctgaaaggccctgggctctgtgctggggacacagcagtgcaaagggtgtcaaagtctctgccttcatggggcttctaatcaagtagagagatacatgcagatggctgcacacaggcttaggtgtgacagggaaggtcaggatgctgtgggagccagaggtgacttccgcttcaccccccacccgcggtggtcccaatctcttccttcctccatgaggtgtctggggtcggggccccagttcttcctggagtccatctggagtctctcctactttctagagaatccattgggtcttgtttacaacgtggatggggacagactgtgcagcgtggagggaaggggagggagggagttttgggaagagcctcctgtggggtccctgtcactgccccagatgcccccaacaccctgtgatacctgcagcccctgccacatctgtccctcactccaaactcagctcaggggatggtggcagggaaggaggtgagcttggacccaggcagccctggggtcaccaccctccagctggggttcctcctctgtaaagtggaggtataacggtacccacctcctggggtggctgtgaggattcagagctgataaggtgaacgcctagggcgggccctggtgcagagagagcgctcagctcctagggctggattaactgtccctggggcacagatctcggtctggggcctgtggaaacctcagagccacccctgaacccccaccgagccaccctttgcctcgcagtgcccatggccttgtctccgaggttacaggaaaaggcagaggagatgcccttctcagggtggccctctgggagaggacactctcccttgacctcaaagccacgcttggctgcaaactggccaggcagccacaaggctgggcaagcaaaactatccctaatccccacccaaagagccacaccgaccctcccagccgctgtgacagctcctgcagagacaaacacacggcctactcttgtcacccgggccggccaataagcacggagaggcaaggcctcagaccctggacagacatcctccctccagaggcacccagggcctcagccttctcctccctccctgggcctcaatttctccacctgtgacccagggcaggtggatccagggagaagaaccttctggctccatctcaccgtgggtcctgccagcacacacaaagatttggcctctcaaagcctagctctgccagcgtccttctgctcaagaactctccatgactcccagtggccctaaggacaaagtcctggcatttgaggccctcccaatgcagggccagactctgcctctccagcttcctgtccccaccacacccctgctggtctcacggtggtccgactgtttcctgcttctgtgcctttgcttagtctggcacccctgcctggcatgctttcctcaccccttcttctccccaatcccaactcacccagtctttcaaagggcaggcctaaataccaggccctccaggtggcccaggattccttctctgagctttcatgggcctggccctgggtgctacctgtgagtagtcccacggtgggtacatagtaggtgcgcttactgtttgcagaatgaacatgggacagtttggggactgtcacccagctcagggagcactgatggggaagcatctcctgtatgtcccagggctcagtgctgtagtgtcctgaccctcagaaatctcataatggcttggtcaggaaggcatcgtgccccactttgcaaacagggggtgctgagaattgaggggccttgtccaaggtctcatggctaggagcaagcagaatcggatttgaacccagggccacgtgacttcagaagtgccattaaagtccccataatttggagctgtcttctttttttttttcttttcttttttttgagaccgagcctcactctgtcacctaggccaggagtgcagtggtctgatctcagctcactgcaacctccgcctcctaggttcaagtgattctctagcctcagcctcccaagtagctgggactacaggcgcacgtcatcatgcccagctaacttttgtatttttagtagagatgggttttcaccatgttggtcaggctggtctcgaactcctgacctcaagtgatccgtctgcctcggcctctcaaagtgctgggattataggcttgagccactacactcggcctggagctgtgttttgtcggtgaaggattttccacccatgaaggggtcagacgtgaagtgtgtggccctgggcagctcctctgagcccagagacgccagccctagccgccttgctgtgccactttgggacttccctccctagcctgagcttcagttttcctgcctgttaggcagccccatgtcaactgcacttagtaggccgggtttgatgcccgacaagacgtgaagtggtggaggtgggcaggatcccagcgctaccatcttcttgaaccagtgatctcaacacatcggatttctgtttcctcatctgcaaaatgggatcagtgagctcaggtgggtcacaaattctacaggaactactttagccaagaccggccccctgaaagttcccctcggtgggctgttagggtgattgttttcatctgtggggctccctgatgcgtcccacccaccagccttggagagggtgggatgggagggtggggtgcttggggagacaagcctagagcctgggccctcccaccccactgcctccccccatcccagggccccccacccagtgacaaagcccgtggcacttcctctacccggttggcaggcggcctggcccagccccttctctaaggaagcgcatttcctgcctccctgggccggccgggctggatgagccaggagctccctgctgccggtcataccacagccttcatctgcgccctggggccaggactgctgctgtcactgccatccattggagcccagcaccccctccccgcccatccttcggacagcaactccagcccagccccgcgtccctgtgtccacttctcctgacccctcggccgccaccccagaaggctggagcagggacgccgtcgctccggccgcctgctcccctcgggtccccgtgcgagcccacgccggccccggtgcccgcccgcagccctgccactggacacaggataaggcccagcgcacaggcccccacgtggacagcatggaccgcggcacgctccctctggctgttgccctgctgctggccagctgcagcctcagccccacaagtaggtgtccagggacccagggtggggagactcggcctccggtgcacggaccaggccccaagtattcccggcctccttcctgtatcctgagctcacgcccagcagagccatccttggggctctggagggtcaccaaccctcccagtttgctggaactaaatggttatgcaggactttcagtgttgaaagaaagcctcgggcaaactgggctgactctttcactttaaccctggtctctggcgtctgctcacccagctgcgttccattactccccgggaagcctaggtcccagaatgctgtgcagcgacgggagagtttcctggcctctctgggccttgagtttccccatgaggaatggatgggaaggagggcccacagcctgggctctgagcaccgtctctgggttcaaatctcacataggcacttccttgtggtgtgatcctgggggactgccttcgggcctctgagcgaagtggggaagagaacagacttccctctcagagctgctgaggaggtgggt

SEQ ID NO: 31 - DNA, 6000 bp, Artificial sequence
  MGST2
actttccaccgtgacacagagataggagcttaaaccttgtactctttttttctacactacattggtaactgatctttattttccttgcctttcttctctttttctgtctttatctttgagccttgatatgtcattactggtgctgggtagtgggcagtttctgctggaaggtagctggcattcaactgtaaccctttgccctgtctggtccagggcaaagtctcctcaatgggaataaaatgcctttgccttctttagctcatttcttcacactcagggctacaaagcaaaatgctgcagtttctgaaatgggagaaaatattccgtaggatgactaaaatttcctaaatttttgcaacatgtagcaaaatggagcataatttcaaattgtgtttatctgagctttgcttaaaaacttactgtgttaaagccactattaaacccagctactcaggagactgaggtgggagggttgcttgaaccctggaagttgaggctgcagtgagccatgatcatgtcactgcactccagccagggtgatagagtaagaccctgtctcaaaacaaaacaaaacacagaaaaactgctgtgtagttacaagattcttcaactagtcaggacttctagcaagataatgtaatgaactctgggtacatggagagttatgtggtcattgataatcatgatcactctgatagttcctgggaaagaggtgtattttctgtctctttctgctacctggcctgatggcagtgaggacagccctgctgtagtgctcagcagaaacctggatgatgtgctgtcaattttggaagggtagcaagtgaacagagagggtgagaccagggaataatttgcgggggttgcaattggtttgaggaaaaacctttttttttttttttttttttttttgagatggagacagagcttggcagagcaagctctgtcacccaggctggagtgttgtagcgcattctcggctcactgcaacttccacctcttgggttcaagtgattctcatgtctcagcctccccagtagctgggattacagatgtgcaccatcatgcctggctaatttttgtatttttagtagagatggggtttcaccatgttggctaggctggtcttgaactcctgacctcaggtgatccacccaccttggcctcccaaagttctgggattacaagtgtgagccaccatgcccggctgaggaaaaacatttctatagtttaatatatcatttcttttcatctattaccaaaattatactgaaagagttacatttagagttcacatttcatgaaagcagttcttttacatggttcaggtttgtttttatgtgatcaaatcttggcctctctgtttcaccttacctagagtatagttttggtaacagtgattcagtagtaaccaaagtcctcagaaagtcttttaggtttcttttgagtttacatagataataaaaatattttaaaaacattttactatcattactgtcttattccattttgttgctataacagaataccacacactgggtaatttataaagaaaagaaatttatttctcacagttctggaggttaggaggtttaatatcaaggtgctggcatctggtaaggacctttgtgctatgtcatcccaaagcgggggggcaagagagggccaagctcacatttataacaatccactcctataacaacattaatccattcatgggagcagagctctcatggcctaatcaccccttattgtcctacctcttaataccattataatggaagttaaattttaacatgagttttggaggggacaaacattcagaccatagcaattaccagtatgctgtttgtcctcttattttccttttcatagaaaaatgtaataaatggaacatactagaaggatctatgggaacaacattcagggaaaatgccattcctcttcaaccaatccagggaaaacgtacagagaaactctaatttttgtgagtttttgttaatgctatggcagtatttcagctgtgggtcacctggaaattttatcactgcagaaggtgataaacatttcattatacaatgctctattcttttatactttcctagaggcaataactacatataccgacaatgaagatctttttaaatagacacgtggggttttgaagcacttgagattttattgattaattgattttttatttttttactttgtgtcaaccatctcttagaatctggatttgggggactgacttaagattggctgggagaagtcagtagggaacctcaggatttgtccagaagctgggaagtttcattttttttttttttttgagacgaagtctcgctctgtcacccaggctggagtgcagtggcacgatctcagctcactgcaagctccgctttcgaggttcacgccattctcctgcctcagcctcccgagtagctgggactacaggcgcccgccaacacgcccggctaatttttttgtatttttagtagagacggggtttcaccgtgttagccaggatggtctggaactcccaacctcgggtgattcgcccacctcggcctcccaaagtgtgggattacaggcgtgagccaccacgcccggccggaagttgtagattttaaaccagttgactagtacaggtgtacccaagagtttctcaaaataagagttaccaacacccaaaagttacagtcatcagccagggccacaattctctgctcagctcctcctgctgttgttcctctcttcccgagccgttctctgactttgagagcctctgcctgtccccaggcctaatgtagacctctccttcgagatctgtcatttgggaggttattatagtagatggtgatatgggttcagggcagctagtttgcagtttagaatctctgccatgtactgaagcccaaaactattaaactgagtatacaagaatgttttggtgctgccccagcttcttggctctactttcttatctaatcttagttgtcaatgatttcaagagcaaacagccaaggaagcctgaagaccaacagaaactttgagcagacctggcttgcaactatttataaattaattttgttattccttctttcctgtatatgtgtagttccatgaattctactttgaactggttataacatcttactggttccctcatgaatgagttacataaaaattttgatatgttttcattttatgttgtatgaaaggcatgatttttgcctctgaaagtattttttaacattaggtacattatatgctaagtacttgtctagatactttatgtgtttattttgcagacacacagaacacatttgtatatgccaggtgctgttttaagtgctctataagtattaactaattcaatccttagaataggattatgtcatttaaacttctcttcagctcttattagccctatcttattaaatggaggctcagagaggcccaaggttgtgttattaaattgtagggtctgtgtgaggctgaggcaactgccctccccagaagacactgggagctccctgctagggactaaatgtttgtgtcccctctgcaattcatatgttgagaccctaacccccaacgtgatgttatcaggcatttggcagataattagctacaggtgaggttatgagcataggcccccataatgggatctgtgcccttataagaagtgaccaggaagcttactctttctctgtctccaacatgaaggtgtccttctgcaaaccaggaagagggccctcaccaggaactgattggccagcactgtcatcttggacttcccagccttcaggactgtgacgaataaattgctattgtaagccacccagtctatggtatctttgttatagcagcttgagccaagatataccctctaatggtgctgtaggatggggtgaggaaaggcccccagctctggtttttgggaagctgcatctttatttttattatttgtttgagacagggtctcactttgtcacccaggctgaagtgcagtggaacgaccttggctcactgcagtctcaggctcctgggttcaagcaatccccttgcctcagccccctaagtagctgggactacaggcacgtgccaccatgccctgctaatttttgtatttctttaagagatggggttttgccatgttacccaggctggtctcgaactcctgagctcaaaccatctgcccatttcagcctcccaaactgttgggattacaggcatgagccatccttccaggccagaatctgcatctctaaagagcagattctcctgcctcagcctcctgcgtagctgggactacaggttcgtgccaccatgcccagctgattttttgtatttttagtagagaaggggttttactgtgttagccaggatagtcttgatctcctgacctcgtgattcaccctccttggcctcttaaagtgctgggattacaagtgtgagccatcgcgcccggccggtttatttttttaaaaatggctgggcacagtgacttgtggctgtagttctagctacttgggaggctgaggcaggaggattacttaagcccaagagttggaggctgcagtgagctatgatcgtgccaccacacactccagcctgggtgacagatcaagaccctgtcttaaaaaaaaaaagtatgattattatcagagtctctaggtgacagtgagaggcagcctggtgcaggggatagggcacagttgtaatctgctagactgggttcaaatcctaaatcggccattcagagcgtgaatacattagaactggcttaaattagcatttaaaatgtcaacaggattaagtaagtagttccttctcactttttccacaggaggtgagaaaggtctcagcaaggccctaggagggaagggcgtggggatgaaagggatccagagagtctgtgcattggcagagaagatagtgtaactggcactgcggctggagggctggtcccacagtgagctacagccctgcccgctggccgtgggagaggcttaaaacaaacgccggaagcaactcccagccccataaagatctgtgaccggcagccccagacctgcctgccttcctgacttctgttccagagcaaaggtcattcagccgcttgaatcagccttttccccccacccggtccccaactttgtttacccgataaggaaggtcagcattcaaagtcaagaagcgccatttatcttcccgtgcgctctacaaatagttccgtgagaaagatggccgggaactcgatcctgctggctgctgtctctattctctcggcctgtcagcaaagtaagaggcatgggaagttcgtgtgtgtgcgcgtgtgtgcgtgtgtgtgtgtgtgtgacaaggcttgcgggagagagagggagggagggagatgggtccggtgttttgtttcctacttgcccttgcaggtagctctgggtcctcagagcacagtcgcctcagggtcacccatgccgcctgctaccctccttcccaggggcaagcagagactgagaacattccagagattagttctcccaactggaacgctgtggggcctcagagctcagcgattctgcatcatctgtgattacgacccacagcccgttcaaacgagcgttagtagcctgctaacctgcaggaagtggtgtgaatattaattacaagtgttccaaaggaaacgtgcctgcttctaaacctggttgtgatttcttgaacgttgatgttttaattaatgtgttttcttaaataaactgcctatggtggtatgattatcagattgaaaaaaacttccttcagaaatattagctttagattaagtaattagctctaaattttaaaacagcttcccactaggattattcaatatctcgactgcctggttaaatagagggcttttactccatgggagtcacatttgctcaattcatattatcttacctacaatgtcagctggggaaaggggtccgagtgcaagagtgcaagacttct

SEQ ID NO: 32 - DNA, 21 bp, Artificial sequence
  ADRA1A
cttagtcatgcccattgggtc

SEQ ID NO: 33 - DNA, 21 bp, Artificial sequence
  BNIP3
tggacggagtagctccaagag

SEQ ID NO: 34 - DNA, 21 bp, Artificial sequence
  C1orf158
gacaagacaccccaatccatt

SEQ ID NO: 35 - DNA, 22 bp, Artificial sequence
  CACNB2
ctatctggaggcctactggaag

SEQ ID NO: 36 - DNA, 21 bp, Artificial sequence
  CACYBP
tctctgtggaaggcagttcaa

SEQ ID NO: 37 - DNA, 24 bp, Artificial sequence
  CEACAM4
cagttacgactctgaccaagcaac

SEQ ID NO: 38 - DNA, 22 bp, Artificial sequence
  HFE2
tcctctttgtccaagccaccag

SEQ ID NO: 39 - DNA, 21 bp, Artificial sequence
  HIST1H3C
gcagcttgctactaaagcagc

SEQ ID NO: 40 - DNA, 19 bp, Artificial sequence
  HS3ST2
gccgtgctggagtttatcc

SEQ ID NO: 41 - DNA, 20 bp, Artificial sequence
  IGSF21
ttcctcaacgtcatggctcc

SEQ ID NO: 42 - DNA, 22 bp, Artificial sequence
  KCNA6-1252F/1467R
gttacaatgaccacggtaggtt

SEQ ID NO: 43 - DNA, 21 bp, Artificial sequence
  MLN
atggtatcccgtaaggctgtg

SEQ ID NO: 44 - DNA, 19 bp, Artificial sequence
  NEFH
cgaggagtggttccgagtg

SEQ ID NO: 45 - DNA, 20 bp, Artificial sequence
  POU4F2-78F/299R
ctcggcactgcacagcacct

SEQ ID NO: 46 - DNA, 25 bp, Artificial sequence
  TWIST1
acttcctctaccaggtcctccagag

SEQ ID NO: 47 - DNA, 21 bp, Artificial sequence
  ADRA1A
ctgcagagacactggattctc

SEQ ID NO: 48 - DNA, 22 bp, Artificial sequence
  BNIP3
ccgacttgaccaatcccatatc

SEQ ID NO: 49 - DNA, 22 bp, Artificial sequence
  C1orf158
tgtttgtaaggtagcccctcaa

SEQ ID NO: 50 - DNA, 22 bp, Artificial sequence
  CACNB2
tcagtcctctgatcaccttgag

SEQ ID NO: 51 - DNA, 23 bp, Artificial sequence
  CACYBP
tctgtttcagtgtcataggaggg

SEQ ID NO: 52 - DNA, 23 bp, Artificial sequence
  CEACAM4
cttccagtcctggagagaagcag

SEQ ID NO: 53 - DNA, 22 bp, Artificial sequence
  HFE2
catcttcaaaggctacaggaag

SEQ ID NO: 54 - DNA, 20 bp, Artificial sequence
  HIST1H3C
cgcacagattggtgtcttcg

SEQ ID NO: 55 - DNA, 21 bp, Artificial sequence
  HS3ST2
ggagcctcttgagtgacaaag

SEQ ID NO: 56 - DNA, 20 bp, Artificial sequence
  IGSF21
cctccagacacgatgcagac

SEQ ID NO: 57 - DNA, 20 bp, Artificial sequence
  KCNA6-1252F/1467R
gtccgttgtcagttgccctc

SEQ ID NO: 58 - DNA, 21 bp, Artificial sequence
  MLN
ctggagttcgccataggtgaa

SEQ ID NO: 59 - DNA, 21 bp, Artificial sequence
  NEFH
gcatagcgtctgtgttcacct

SEQ ID NO: 60 - DNA, 20 bp, Artificial sequence
  POU4F2-78F/299R
actctcatccagcccgccga

SEQ ID NO: 61 - DNA, 25 bp, Artificial sequence
  TWIST1
acaatgacatctaggtctccggccc

SEQ ID NO: 62 - DNA, 29 bp, Artificial sequence
  ADRA1A_py06
tttaggtggggtagtttaaaatgtaggta

SEQ ID NO: 63 - DNA, 18 bp, Artificial sequence
  BNIP3_py03
tgggagaggggtagaggt

SEQ ID NO: 64 - DNA, 18 bp, Artificial sequence
  BNIP3_py05
tgggagaggggtagaggt

SEQ ID NO: 65 - DNA, 22 bp, Artificial sequence
  BNIP3_py07
gggttgagggatgtgttttagt

SEQ ID NO: 66 - DNA, 21 bp, Artificial sequence
  C1orf158_py04
ggaggatgaggtaggagaatg

SEQ ID NO: 67 - DNA, 24 bp, Artificial sequence
  CACNB2_py04,05,06
gttgtgggaggagatttggatatg

SEQ ID NO: 68 - DNA, 21 bp, Artificial sequence
  CACYBP_03,04
aggagaaaaatggggaggagt

SEQ ID NO: 69 - DNA, 22 bp, Artificial sequence
  CD248_py02
gggtaagaaaggagtgggtatg

SEQ ID NO: 70 - DNA, 23 bp, Artificial sequence
  CD248_py03,04
ttttaggggaagagggagtaggg

SEQ ID NO: 71 - DNA, 19 bp, Artificial sequence
  HS3ST2_py02,03,04
agggggagggttaggtttt

SEQ ID NO: 72 - DNA, 24 bp, Artificial sequence
  HS3ST2_py06
aggatagggagatgttggaaatgt

SEQ ID NO: 73 - DNA, 30 bp, Artificial sequence
  IGSF21_py01
atgagggtatttatagttggtaaggttaga

SEQ ID NO: 74 - DNA, 25 bp, Artificial sequence
  IGSF21_py02
aagaagttggaggtagtaagttagt

SEQ ID NO: 75 - DNA, 25 bp, Artificial sequence
  KCNA6_py01
gggaaaggtattgattgatttgtta

SEQ ID NO: 76 - DNA, 25 bp, Artificial sequence
  MLN_py02
gttttagggggaagattgaagagaa

SEQ ID NO: 77 - DNA, 24 bp, Artificial sequence
  MLN_py07
tttagggttgggaggtatataaga

SEQ ID NO: 78 - DNA, 18 bp, Artificial sequence
  NEFH_py05
gtgagagggtggggagga

SEQ ID NO: 79 - DNA, 24 bp, Artificial sequence
  NEFH_py07
gagtggaagtagttggaggagtta

SEQ ID NO: 80 - DNA, 24 bp, Artificial sequence
  OR2L13_py05
agggttatttgtaatgtgggtaag

SEQ ID NO: 81 - DNA, 24 bp, Artificial sequence
  POU4F2_py06,07
gttggaggttggtttttaggtagg

SEQ ID NO: 82 - DNA, 20 bp, Artificial sequence
  TBX20_py05,07
ggtggggaatagaggttagt

SEQ ID NO: 83 - DNA, 29 bp, Artificial sequence
  TWIST1_py04
tgggagagatgagatattatttattgtgt

SEQ ID NO: 84 - DNA, 27 bp, Artificial sequence
  ADRA1A_py06
ccttacaacatacaattccaaaattac

SEQ ID NO: 85 - DNA, 19 bp, Artificial sequence
  BNIP3_py03
cctcaatttccccactaac

SEQ ID NO: 86 - DNA, 21 bp, Artificial sequence
  BNIP3_py05
atcccacccccccttcaaaaa

SEQ ID NO: 87 - DNA, 19 bp, Artificial sequence
  BNIP3_py07
accccaaacctctacccct

SEQ ID NO: 88 - DNA, 30 bp, Artificial sequence
  C1orf158_py04
aaaactccaaaaaactatatattccatctt

SEQ ID NO: 89 - DNA, 23 bp, Artificial sequence
  CACNB2_py04,05,06
acccccctaaaaactcccctctc

SEQ ID NO: 90 - DNA, 28 bp, Artificial sequence
  CACYBP_03,04
cccttttattaaaaccttaacctaaact

SEQ ID NO: 91 - DNA, 25 bp, Artificial sequence
  CD248_py02
ccaaaccccataaaactaaaaatca

SEQ ID NO: 92 - DNA, 28 bp, Artificial sequence
  CD248_py03,04
caacaacccaaaaatcctaacccaatat

SEQ ID NO: 93 - DNA, 21 bp, Artificial sequence
  HS3ST2_py02,03,04
attacatttccaacatctccc

SEQ ID NO: 94 - DNA, 21 bp, Artificial sequence
  HS3ST2_py06
acccaaaaccctataaaccat

SEQ ID NO: 95 - DNA, 21 bp, Artificial sequence
  IGSF21_py01
cccctcactcaaaactaactt

SEQ ID NO: 96 - DNA, 19 bp, Artificial sequence
  IGSF21_py02
ccccccccctccttaccct

SEQ ID NO: 97 - DNA, 24 bp, Artificial sequence
  KCNA6_py01
taccaacctctccaatatctacaa

SEQ ID NO: 98 - DNA, 24 bp, Artificial sequence
  MLN_py02
acccattaacctttaaccacaact

SEQ ID NO: 99 - DNA, 24 bp, Artificial sequence
  MLN_py07
cacccacaacaacctctactttac

SEQ ID NO: 100 - DNA, 23 bp, Artificial sequence
  NEFH_py05
catcctacccctattcccatcaa

SEQ ID NO: 101 - DNA, 26 bp, Artificial sequence
  NEFH_py07
accctctcactaccaaaaaattaaac

SEQ ID NO: 102 - DNA, 24 bp, Artificial sequence
  OR2L13_py05
caaaaattttcctacccaaaaact

SEQ ID NO: 103 - DNA, 24 bp, Artificial sequence
  POU4F2_py06,07
ctactcccctcaaacttaaatcct

SEQ ID NO: 104 - DNA, 21 bp, Artificial sequence
  TBX20_py05,07
aacccaacttacccaaaaatt

SEQ ID NO: 105 - DNA, 29 bp, Artificial sequence
  TWIST1_py04
tctaacaattcctcctcccaaaccattca

SEQ ID NO: 106 - DNA, 19 bp, Artificial sequence
  HIST1H2BN
ttcgggggtgggagagagc

SEQ ID NO: 107 - DNA, 18 bp, Artificial sequence
  ATG4A
ggggttttcgttagggtc

SEQ ID NO: 108 - DNA, 16 bp, Artificial sequence
  THRB
acgggtcgggtcggtc

SEQ ID NO: 109 - DNA, 22 bp, Artificial sequence
  STC2
cgggaaaggaaagttttggaag

SEQ ID NO: 110 - DNA, 22 bp, Artificial sequence
  ENG
cgtttgtttttttcgggttttc

SEQ ID NO: 111 - DNA, 23 bp, Artificial sequence
  MGST2
aagcgttatttattttttcgtgc

SEQ ID NO: 112 - DNA, 24 bp, Artificial sequence
  HIST1H2BN
acaaaaaacatacacacacgcacg

SEQ ID NO: 113 - DNA, 19 bp, Artificial sequence
  ATG4A
ctaaatctctccgcaatcg

SEQ ID NO: 114 - DNA, 20 bp, Artificial sequence
  THRB
cacccacccgattacctacg

SEQ ID NO: 115 - DNA, 22 bp, Artificial sequence
  STC2
acgaaaaaacacgcgaacaaat

SEQ ID NO: 116 - DNA, 22 bp, Artificial sequence
  ENG
ctaatccgtacaccgaaaaccg

SEQ ID NO: 117 - DNA, 17 bp, Artificial sequence
  MGST2
cacgcgcacacacacga

SEQ ID NO: 118 - DNA, 26 bp, Artificial sequence
  HIST1H2BN
agtattatattttagggggtgggaga

SEQ ID NO: 119 - DNA, 23 bp, Artificial sequence
  ATG4A
gggaaaatatttgaggtttgtgg

SEQ ID NO: 120 - DNA, 29 bp, Artificial sequence
  THRB
ggattagaggaggttttaagaagagttag

SEQ ID NO: 121 - DNA, 22 bp, Artificial sequence
  STC2
gggaaaggaaagttttggaagt

SEQ ID NO: 122 - DNA, 28 bp, Artificial sequence
  ENG
ggtagttattttagaaggttggagtagg

SEQ ID NO: 123 - DNA, 19 bp, Artificial sequence
  MGST2
ggttggagggttggtttta

SEQ ID NO: 124 - DNA, 25 bp, Artificial sequence
  HIST1H2BN
acaaaccaatttaaaaaacaactct

SEQ ID NO: 125 - DNA, 27 bp, Artificial sequence
  ATG4A
ccctaactactaaaactaaccaaataa

SEQ ID NO: 126 - DNA, 24 bp, Artificial sequence
  THRB
ctccccacctacctccccaaatat

SEQ ID NO: 127 - DNA, 20 bp, Artificial sequence
  STC2
aaatttcatcacccactacc

SEQ ID NO: 128 - DNA, 27 bp, Artificial sequence
  ENG
ccctaaatccctaaacacctacttata

SEQ ID NO: 129 - DNA, 27 bp, Artificial sequence
  MGST2
acaccaacttcccatacctcttacttt

(19) United States
(12) Patent Application Publication
(10) Pub. No.: US 2015/0072947 A1
(43) Pub. Date: Mar. 12, 2015

(54) GENE BIOMARKERS FOR PREDICTION OF SUSCEPTIBILITY OF OVARIAN NEOPLASMS AND/OR PROGNOSIS OR MALIGNANCY OF OVARIAN CANCERS

(71) Applicants: National Defense Medical Center, Taipei City (TW); DCB-USA LLC, Wilmington, DE (US)

(72) Inventors: Hung-Cheng Lai, Taipei City (TW); Rui-Lan Huang, Taipei City (TW)

(73) Assignee: National Defense Medical Center, Taipei City (TW)

(21) Appl. No.: 14/241,803

(22) PCT Filed: Aug. 30, 2012
(86) PCT No.: PCT/US12/53050

§ 371 Date: Aug. 25, 2014

Related U.S. Application Data
(60) Provisional application No. 61/528,805, filed on Aug. 30, 2011.

(51) Int. Cl. C12Q 1/68

(52) U.S. Cl. CPC C12Q 1/6886; C12Q 2600/154; C12Q 2600/106
USPC 514/49; 435/6.11; 514/43; 514/535; 514/562

(57) ABSTRACT

The present invention uses methylomic analysis and discovers DNA methylation biomarkers for prediction of ovarian cancer prognosis and detection of malignant ovarian cancer. In addition to being independent prognostic factors for patients with current treatment protocols, these DNA methylations are important biomarkers for individualized medicine for future chemotherapy (especially the demethylation agents or other epigenetic drugs).

### FIELD OF THE INVENTION

The invention relates to gene biomarkers for prediction of risk or susceptibility of ovarian neoplasms and/or prognosis and malignancy of ovarian cancers. In particular, the invention uses DNA methylation to select candidate genes for prediction of susceptibility of ovarian neoplasms and/or prognosis and malignancy of ovarian cancers.

### BACKGROUND OF THE INVENTION

Ovarian cancer is a serious disease which causes more deaths than any other cancer of the female reproductive system. Because of the insidious onset of the disease and the lack of reliable screening tests, two thirds of patients have advanced disease when diagnosed, and although many patients with disseminated tumors respond initially to standard combinations of surgical and cytotoxic therapy, nearly 90 percent will develop recurrence and inevitably succumb to their disease. Understanding the molecular basis of ovarian cancer may have the potential to significantly refine diagnosis and management of the cancer, and may eventually lead to the development of novel, more specific and more effective treatment modalities. There is a need for better prognostic indicators to guide the vigor and extent of surgical and adjuvant therapies, especially in patients at early stage of the disease.

DNA methylation is one of the epigenetic mechanisms that plays a role in many important biological processes including X-inactivation, silencing parasitic DNA elements, genomic imprinting, aging, male infertility, and cancer. DNA methylation involves a post-replication modification predominantly found in cytosines of the dinucleotide CpG that is infrarepresented throughout the genome except at small regions named CpG islands. Previous studies have shown CpG island DNA hypermethylation in various cancers, including ovarian tumors, as well as reduced levels of global DNA methylation associated with cancer. The pattern of DNA methylation in a given cell appears to be associated with the stability of gene expression states. It is known in the art that changes in CpG methylation are cumulative with ovarian cancer progression in a sequence-type dependent manner, and that CpG island microarrays can rapidly discover novel genes affected by CpG methylation in clinical samples of ovarian cancer (George S Watts et al., “DNA methylation changes in ovarian cancer are cumulative with disease progression and identify tumor stage,” BMC Medical Genomics 2008, 1:47). Caroline A. Barton et al., which provides the detection of cancer-specific DNA methylation changes, heralds an exciting new era in cancer diagnosis as well as evaluation of prognosis and therapeutic responsiveness and warrants further investigation (Caroline A. Barton et al., “DNA methylation changes in ovarian cancer: Implications for early diagnosis, prognosis and treatment”, Gynecologic Oncology, Volume 109, Issue 1, April 2008, pages 129-139). Sahar Houshdaran et al. indicates that the distinct methylation profiles of the different histological types of ovarian tumors reinforces the need to treat the different histologies of ovarian cancer as different diseases, both clinically and in biomarker studies (Sahar Houshdaran et al., “DNA Methylation Profiles of Ovarian Epithelial Carcinoma Tumors and Cell Lines”; PLoS ONE, Volume 5, Issue 2, February 2010, e9359). U.S. Pat. No. 7,507,536 provides twenty-three markers which are epigenetically silenced in ovarian cancers and these markers can be used diagnostically, prognostically, therapeutically, and for selecting treatments that are well tailored for an individual patient.

However, the roles of cumulated hypermethylation and hypomethylation in ovarian cancer progression and outcome are still unknown. There remains a need to develop biomarkers for predicting prognosis of ovarian cancer on the basis of DNA methylation.

### SUMMARY OF THE INVENTION

The invention relates to a method of predicting risk or susceptibility of ovarian neoplasms in a subject, comprising assessing DNA methylation of one or more of the following genes in an ovarian neoplasm sample obtained from said subject: NPTX2, TNNI1, POU4F2, HS3ST2, CACNB2, TBX20, OR2L13, IGSF21, CD248, ADRA1A, NEFH, BNIP3, C1QTNF3, KCNA6, CEACAM4, CRNN, HFE2, TWIST1, GATA4, CACYBP, HIST1H2AJ, C1orf158, A4GALT, MLN, HIST1H3C, STC2, ATG4A, ENG, HIST1H2BN, MGST2 and THRB, or a polynucleotide sequence with at least 80% similarity thereof; wherein change of DNA methylation indicates that the subject is susceptible of ovarian neoplasms.

The invention also relates to a method of predicting prognosis or malignancy in a subject diagnosed with an ovarian neoplasm, comprising assessing DNA methylation of one or more of the following genes in an ovarian cancer sample obtained from said subject: NPTX2, TNNI1, POU4F2, HS3ST2, CACNB2, TBX20, OR2L13, IGSF21, CD248, ADRA1A, NEFH, BNIP3, C1QTNF3, KCNA6, CEACAM4, CRNN, HFE2, TWIST1, GATA4, CACYBP, HIST1H2AJ, C1orf158, A4GALT, MLN, HIST1H3C, STC2, ATG4A, ENG, HIST1H2BN, MGST2 and THRB, or a polynucleotide sequence with at least 80% similarity thereof; wherein change of DNA methylation indicates a poor prognosis or a malignant ovarian cancer.

The invention also relates to a method of detecting prognosis or malignancy in a subject diagnosed with ovarian cancer comprising assessing DNA methylation of one or more of the following genes in an ovarian cancer sample obtained from said subject: NPTX2, TNNI1, POU4F2, HS3ST2, CACNB2, TBX20, OR2L13, IGSF21, CD248, ADRA1A, NEFH, BNIP3, C1QTNF3, KCNA6, CEACAM4, CRNN, HFE2, TWIST1, GATA4, CACYBP, HIST1H2AJ, C1orf158, A4GALT, MLN, HIST1H3C, STC2, ATG4A, ENG, HIST1H2BN, MGST2 and THRB, or a polynucleotide sequence with at least 80% similarity thereof; wherein DNA hypermethylation of one or more of NPTX2, TNNI1, POU4F2, HS3ST2, CACNB2, TBX20, OR2L13, IGSF21, CD248, ADRA1A, NEFH, BNIP3, C1QTNF3, KCNA6, CEACAM4, CRNN, HFE2, TWIST1, GATA4, ATG4A, HIDT1H2BN, THRB and MGST2, as compared to DNA methylation observed in non-cancer cells, and/or DNA hypomethylation of one or more of CACYBP, HIST1H2AJ, C1orf158, A4GALT, MLN, HIST1H3C, STC2 and ENG, as compared to DNA methylation observed in non-cancer cells, indicates a poor prognosis or a malignant ovarian cancer.

The invention also relates to a method of making a treatment decision for a subject with ovarian cancer, comprising administering an effective amount of a demethylating agent to the subject, wherein the subject exhibits DNA hypermethylation of one or more of NPTX2, TNNI1, POU4F2, HS3ST2, CACNB2, TBX20, OR2L13, IGSF21, CD248, ADRA1A, NEFH, BNIP3, C1QTNF3, KCNA6, CEACAM4, CRNN, HFE2, TWIST1, GATA4, ATG4A, HIDT1H2BN, THRB and MGST2, or a polynucleotide sequence with at least 80% similarity thereof, as compared to DNA methylation observed in non-cancer cells.

The invention further relates to a method of determining a therapeutic regimen for a subject having a poor prognosis or malignancy in ovarian cancer, comprising providing chemotherapy to the subject, wherein the subject has DNA hypermethylation of one or more of NPTX2, TNNI1, POU4F2, HS3ST2, CACNB2, TBX20, OR2L13, IGSF21, CD248, ADRA1A, NEFH, BNIP3, C1QTNF3, KCNA6, CEACAM4, CRNN, HFE2, TWIST1, GATA4, ATG4A, HIDT1H2BN, THRB and MGST2, or a polynucleotide sequence with at least 80% similarity thereof, as compared to DNA methylation observed in non-cancer cells, and/or DNA hypomethylation of one or more of CACYBP, HIST1H2AJ, C1orf158, A4GALT, MLN, HIST1H3C, STC2 and ENG, as compared to DNA methylation observed in non-cancer cells.

The invention also further relates to a kit for predicting risk or susceptibility of ovarian neoplasms or a prognosis, detecting malignancy and/or making a treatment decision for a subject with ovarian cancer, comprising reagents for differentiating methylated and non-methylated cytosine residues of one or more of the genes NPTX2, TNNI1, POU4F2, HS3ST2, CACNB2, TBX20, OR2L13, IGSF21, CD248, ADRA1A, NEFH, BNIP3, C1QTNF3, KCNA6, CEACAM4, CRNN, HFE2, TWIST1, GATA4, CACYBP, HIST1H2AJ, C1orf158, A4GALT, MLN, HIST1H3C, STC2, ATG4A, ENG, HIST1H2BN, MGST2 and THRB, or a polynucleotide sequence with at least 80% similarity thereof; wherein DNA hypermethylation of one or more of NPTX2, TNNI1, POU4F2, HS3ST2, CACNB2, TBX20, OR2L13, IGSF21, CD248, ADRA1A, NEFH, BNIP3, C1QTNF3, KCNA6, CEACAM4, CRNN, HFE2, TWIST1, GATA4, ATG4A, HIDT1H2BN, THRB and MGST2, as compared to DNA methylation observed in non-cancer cells, and/or DNA hypomethylation of one or more of CACYBP, HIST1H2AJ, C1orf158, A4GALT, MLN, HIST1H3C, STC2 and ENG, as compared to DNA methylation observed in non-cancer cells, indicates a poor prognosis or malignancy in ovarian cancer.

### BRIEF DESCRIPTION OF THE DRAWING

FIG. 1 shows the volvano plot illustrating the differential methylation in microarray.

FIG. 2 shows the histogram illustrating the risk ratio (hazard ratio, HR) of methylation of twenty five genes using univariate COX proportional hazard regression analysis. a) DNA hypermethylation with poor prognosis listed at right side and DNA hypomethylation with poor prognosis listed at the left side. b) Kaplan-Meier survival estimation of overall survival in patients with ovarian carcinoma. c) shows Kaplan-meier survival estimates of the progression-free survival (PFS) in patients with ovarian carcinoma.

FIG. 3 shows Kaplan-Meier plots of the probability of progression-free survival (A)(B)(E) and overall survival (C)(D)(F) in ovarian cancer patients. Progression-free survival and overall survival stratified by the methylation status of ATG4A and HIST1H2BN are shown for ovarian cancer patients as estimated by Kaplan-Meier curves and the log-rank test. Straight line: high methylation; bold line: low methylation. The low methylation defined as both genes low methylated and high methylation as at least one gene methylated at (E)(F).

FIG. 4 shows the promoter methylation status of ATG4A (A) and HIST1H2BN (B) determined by qMSP in ovarian tissues. *p<0.05.

### DETAILED DESCRIPTION OF THE INVENTION

The present invention uses methylomic analysis and discovers DNA methylation biomarkers for prediction of risk or susceptibility of ovarian neoplasms and/or ovarian cancer prognosis and detection of malignant ovarian cancer. In addition to being independent prognostic factors for patients with current treatment protocols, these DNA methylations are important biomarkers for individualized medicine for future chemotherapy (especially the demethylation agents or other epigenetic drugs).

It is understood that this invention is not limited to the particular materials and methods described herein. It is also to be understood that the terminology used herein is for the purpose of describing particular embodiments and is not intended to limit the scope of the present invention which will be limited only by the appended claims.

As used herein, the singular forms “a”, “an”, and “the” include plural reference unless the context clearly dictates otherwise.

As used herein, the term “biomarker” refers to a nucleic acid molecule which is present in a sample taken from patients having human cancer as compared to a comparable sample taken from control subjects (e.g., a person with a negative diagnosis or undetectable cancer, normal or healthy subject).

As used herein, the term “prediction” refers to the likelihood that a patient will respond either favorably or unfavorably to a drug or set of drugs, and also the extent of those responses. Thus, treatment predictive factors are variables related to the response of an individual patient to a specific treatment, independent of prognosis.

As used herein, the term “epigenetic state” or “epigenetic status” refers to any structural feature at a molecular level of a nucleic acid (e.g., DNA or RNA) other than the primary nucleotide sequence. For instance, the epigenetic state of a genomic DNA may include its secondary or tertiary structure determined or influenced by, e.g., its methylation pattern or its association with cellular proteins.

As used herein, the term “methylation profile” or “methylation status” refers to a presentation of methylation status of one or more cancer marker genes in a subject's genomic DNA. In some embodiments, the methylation profile is compared to a standard methylation profile comprising a methylation profile from a known type of sample (e.g., cancerous or non-cancerous samples or samples from different stages of cancer). In some embodiments, methylation profiles are generated using the methods of the present invention. The profile may be in a graphical representation (e.g., on paper or on a computer screen), a physical representation (e.g., a gel or array) or a digital representation stored in computer memory.

As used herein, the term “hypermethylation” refers to the average methylation state corresponding to an increased presence of 5-mCyt at one or a plurality of CpG dinucleotides within a DNA sequence of a test DNA sample, relative to the amount of 5-methylcytosine (5-mCyt) found at corresponding CpG dinucleotides within a normal control DNA sample.

As used herein, the term “hypomethylation” refers to the average methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample.

As used herein, the term “subject” shall mean any animal, such as a mammal, and shall include, without limitation, mice and humans.

As used herein, the term “neoplasm” refers to an abnormal mass of tissue as a result of neoplasia. Neoplasia is the abnormal proliferation of cells. The growth of neoplastic cells exceeds and is not coordinated with that of the normal tissues around it. The growth persists in the same excessive manner even after cessation of the stimuli. It usually causes a lump or tumor. Neoplasms may be benign, pre-malignant (carcinoma in situ) or malignant (cancer). According to the invention, the neoplasm sample is a sample obtained from a subject, preferably a human subject, or present within a subject, preferably a human subject, including a tissue, tissue sample, or cell sample (e.g., a tissue biopsy, for example, an aspiration biopsy, a brush biopsy, a surface biopsy, a needle biopsy, a punch biopsy, an excision biopsy, an open biobsy, an incision biopsy or an endoscopic biopsy), tumor, tumor sample, or biological fluid (e.g., peritoneal fluid, blood, serum, lymph, spinal fluid).

As used herein, the term “susceptibility” refers to a constitution or condition of the body which makes the tissues react in special ways to certain extrinsic stimuli and thus tends to make the individual more than usually susceptible to certain diseases.

As used herein, the term “risk” refers to the estimated chance of getting a disease during a certain time period, such as within the next 10 years, or during the lifetime.

As used herein, the term “tumor cell” shall mean a cancerous cell within, or originating from, a tumor. Tumor cells are distinct from other, non-cancerous cells present in a tumor, such as vascular cells.

As used herein, the term “prognosis” refers to the prediction of the likelihood of cancer-attributable death or progression, including recurrence, metastatic spread, and drug resistance, of a neoplastic disease, such as ovarian cancer.

As used herein, the term “microarray” refers to an ordered arrangement of hybridizable array elements, preferably polynucleotide probes, on a substrate.

As used herein, the term “detect” or “detection” refers to identifying the presence, absence or amount of the object to be detected.

As used herein, the term “treatment” is an intervention performed with the intention of preventing the development or altering the pathology or symptoms of a disorder. Accordingly, “treatment” refers to both therapeutic treatment and prophylactic or preventative measures.

In one aspect, the invention provides a method of predicting risk or susceptibility of ovarian neoplasms in a subject, comprising assessing DNA methylation of one or more of the following genes in an ovarian neoplasm sample obtained from said subject: NPTX2, TNNI1, POU4F2, HS3ST2, CACNB2, TBX20, OR2L13, IGSF21, CD248, ADRA1A, NEFH, BNIP3, C1QTNF3, KCNA6, CEACAM4, CRNN, HFE2, TWIST1, GATA4, CACYBP, HIST1H2AJ, C1orf158, A4GALT, MLN, HIST1H3C, STC2, ATG4A, ENG, HIST1H2BN, MGST2 and THRB, or a polynucleotide sequence with at least 80% similarity thereof; wherein change of DNA methylation indicates that the subject is susceptible of ovarian neoplasms. Preferably, the gene with DNA methylation is ATG4A, HIST1H2BN, ADRA1A, CACNB2, GATA4, KCNA6, POU4F2, HS3ST2, NEFH, CACYBP or C1orf158 or any combination thereof. More preferably, the gene with DNA methylation is ATG4A, HIST1H2BN, ADRA1A, CACNB2, GATA4, KCNA6, POU4F2, HS3ST2 or NEFH or any combination thereof. More preferably, the gene with DNA methylation is ATG4A, HIST1H2BN, CEACAM4, GATA4 or IGSF21 or any combination thereof. More preferably, the gene with DNA methylation is CEACAM4, GATA4 or IGSF21 or any combination thereof. More preferably, the gene with DNA methylation is POU4F2, NEFH, HS3ST2 or any combination thereof. More preferably, the gene with DNA methylation is CACYBP, or MLN or a combination thereof.

In another aspect, the invention provides a method of predicting prognosis or malignancy in a subject diagnosed with an ovarian cancer, comprising assessing DNA methylation of one or more of the following genes in an ovarian cancer sample obtained from said subject: NPTX2, TNNI1, POU4F2, HS3ST2, CACNB2, TBX20, OR2L13, IGSF21, CD248, ADRA1A, NEFH, BNIP3, C1QTNF3, KCNA6, CEACAM4, CRNN, HFE2, TWIST1, GATA4, CACYBP, HIST1H2AJ, C1orf158, A4GALT, MLN, HIST1H3C, STC2, ATG4A, ENG, HIST1H2BN, MGST2 and THRB, or a polynucleotide sequence with at least 80% similarity thereof; wherein change of DNA methylation indicates a poor prognosis or a malignant ovarian cancer. Preferably, the gene with DNA methylation is ATG4A, HIST1H2BN, ADRA1A, CACNB2, GATA4, KCNA6, POU4F2, HS3ST2, NEFH, CACYBP or C1orf158 or any combination thereof. More preferably, the gene with DNA methylation is ATG4A, HIST1H2BN, ADRA1A, CACNB2, GATA4, KCNA6, POU4F2, HS3ST2 or NEFH or any combination thereof. More preferably, the gene with DNA methylation is CEACAM4, GATA4 or IGSF21 or any combination thereof. More preferably, the gene with DNA methylation is POU4F2, NEFH, HS3ST2 or any combination thereof. More preferably, the gene with DNA methylation is CACYBP, or MLN or a combination thereof.

In one embodiment, the invention provides a method of predicting prognosis or malignancy in a subject diagnosed with ovarian cancer comprising assessing DNA methylation of one or more of the following genes in an ovarian cancer sample obtained from said subject: NPTX2, TNNI1, POU4F2, HS3ST2, CACNB2, TBX20, OR2L13, IGSF21, CD248, ADRA1A, NEFH, BNIP3, C1QTNF3, KCNA6, CEACAM4, CRNN, HFE2, TWIST1, GATA4, CACYBP, HIST1H2AJ, C1orf158, A4GALT, MLN, HIST1H3C, STC2, ATG4A, ENG, HIST1H2BN, MGST2 and THRB, or a polynucleotide sequence with at least 80% similarity thereof; wherein DNA hypermethylation of one or more of NPTX2, TNNI1, POU4F2, HS3ST2, CACNB2, TBX20, OR2L13, IGSF21, CD248, ADRA1A, NEFH, BNIP3, C1QTNF3, KCNA6, CEACAM4, CRNN, HFE2, TWIST1, GATA4, ATG4A, HIST1H2BN, THRB and MGST2, as compared to DNA methylation observed in non-cancer cells, and/or DNA hypomethylation of one or more of CACYBP, HIST1H2AJ, C1orf158, A4GALT, MLN, HIST1H3C, STC2 and ENG, as compared to DNA methylation observed in non-cancer cells, indicates a poor prognosis or a malignant ovarian cancer. Preferably, the gene with DNA hypermethylation is ATG4A, HIST1H2BN, ADRA1A, CACNB2, GATA4, KCNA6, POU4F2, HS3ST2 or NEFH or any combination thereof. More preferably, the gene with DNA hypermethylation is ATG4A, HIST1H2BN, CEACAM4, GATA4 or IGSF21 or any combination thereof. More preferably, the gene with DNA hypermethylation is POU4F2, NEFH, HS3ST2 or any combination thereof. More preferably, the gene with DNA hypermethylation is CEACAM4, GATA4 or IGSF21 or any combination thereof. Preferably, the gene with DNA hypomethylation is CACYBP or C1orf158 or any combination thereof.

The invention compares the methylation profiles of subjects with different survival outcomes to select candidate genes as biomarkers for risk or susceptibility of ovarian neoplasms and/or prognosis prediction and/or detection of malignant ovarian cancers. These aims are achieved by the analysis of the CpG methylation status of at least one or a plurality of genes.

Particular embodiments of the present invention provide a novel application of the analysis of methylation levels and/or patterns of genes that enable a precise prognosis of ovarian cancer and thereby enable the improved treatment. The invention is particularly preferred for the prediction of prognosis and detection of malignancy of ovarian cancer. The method enables the physician and patient to make better and more informed treatment decisions. These aims are achieved by the analysis of the CpG methylation status of at least one or a plurality of genes.

According to the invention, prognosis may be length of survival, such as disease-specific length of survival or overall survival. Prognosis may alternatively be length of time to recurrence.

DNA methylation is a chemical modification of DNA performed by enzymes called methyltransferases, in which a methyl group (m) is added to certain cytosines (C) of DNA. This non-mutational (epigenetic) process (mC) is a critical factor in gene expression regulation. DNA methylation has also been shown to be a common alteration in cancer leading to elevated or decreased expression of a broad spectrum of genes (Jones, P. A., Cancer Res. 65:2463 (1996)). Because DNA methylation correlates with the level of specific gene expression in many cancers, it serves as a useful surrogate to expression profiling of tumors (Toyota, M. et al., Blood 97: 2823 (2001), Adorjan, P. et al. Nucl. Acids. Res. 10:e21 (2002)). By performing differential methylation analysis, the invention has discovered a set of genes exhibiting DNA hypermethylation or DNA or hypomethylation which indicates risk or susceptibility of ovarian neoplasms and/or a poor prognosis in ovarian cancer and/or malignancy in ovarian cancer. These genes and their sequences are listed in the table below:

| No. | Gene name | Sequence |
|---|---|---|
| 1. | C1orf158 | SEQ ID NO: 1 |
| 2. | IGSF21 | SEQ ID NO: 2 |
| 3. | HFE2 | SEQ ID NO: 3 |
| 4. | CRNN | SEQ ID NO: 4 |
| 5. | CACYBP_{—} | SEQ ID NO: 5 |
| 6. | OR2L13 | SEQ ID NO: 6 |
| 7. | CACNB2 | SEQ ID NO: 7 |
| 8. | BNIP3 | SEQ ID NO: 8 |
| 9. | CD248 | SEQ ID NO: 9 |
| 10. | KCNA6 | SEQ ID NO: 10 |
| 11. | HS3ST2 | SEQ ID NO: 11 |
| 12. | CEACAM4 | SEQ ID NO: 12 |
| 13. | NEFH | SEQ ID NO: 13 |
| 14. | A4GALT | SEQ ID NO: 14 |
| 15. | POU4F2 | SEQ ID NO: 15 |
| 16. | C1QTNF3 | SEQ ID NO: 16 |
| 17. | HIST1H3C | SEQ ID NO: 17 |
| 18. | HIST1H2AJ | SEQ ID NO: 18 |
| 19. | MLN | SEQ ID NO: 19 |
| 20. | TWIST1 | SEQ ID NO: 20 |
| 21. | NPTX2 | SEQ ID NO: 21 |
| 22. | GATA4 | SEQ ID NO: 22 |
| 23. | ADRA1A | SEQ ID NO: 23 |
| 24. | TNNI1 | SEQ ID NO: 24 |
| 25. | TBX20_{—} | SEQ ID NO: 25 |
| 26 | ATG4A | SEQ ID NO: 26 |
| 27 | HIST1H2BN | SEQ ID NO: 27 |
| 28. | THRB | SEQ ID NO: 28 |
| 29. | STC2 | SEQ ID NO: 29 |
| 30. | ENG | SEQ ID NO: 30 |
| 31. | MGST2 | SEQ ID NO: 31 |

Among the genes in the above table, there are no prior art describing that C1orf158, CACNB2, CACYBP, IGSF21, KCNA6, OR2L13, TBX20, MLN, ATG4A, HIST1H2BN, THRB, STC2, ENG and MGST2 are associated with cancer and gene methylation. Several prior references disclose that A4GALT (J Biol Chem. 2002 Mar. 29; 277(13):11247-54. Epub 2002 Jan. 8; BMB Rep. 2009 May 31; 42(5):310-4), ADRA1A (PLoS One. 2009 Sep. 18; 4(9):e7068; PLoS One. 2008; 3(11):e3742. Epub 2008 Nov. 17) and CD248 (BMC Cancer. 2009 Nov. 30; 9:417) are associated with cancers other than ovarian cancer. Some prior references reported that HS3ST2 (Oncogene. 2003 Jan. 16; 22(2):274-80) and TWIST1 (Cancer Prev Res (Phila). 2010 Sep.; 3(9):1053-5. Epub 2010 Aug. 10) are associated with gene methylation. Some prior references disclose that BNIP3 (Tumori. 2010 January-February; 96(1):138-42; BMC Cancer. 2009 Jun. 9; 9:175; World J Gastroenterol. 2010 Jan. 21; 16(3):330-8) and NEFH (PLoS One. 2010 Feb. 3; 5(2):e9003; Cancer. 2009 Aug. 1; 115(15):3412-26), POU4F2 (Oncogene. 2008 Jan. 3; 27(1):145-54. Epub 2007 Jul. 16; FEBS Lett. 2007 May 29; 581(13):2490-6. Epub 2007 May 2; BMC Med Genomics. 2009 Aug. 17; 2:53) are associated with cancers and methylation other than ovarian cancer.

Although hypermethylation or hypomethylation is commonly known in a wide variety of cancers, it has not been widely investigated as a prognostic marker and hypermethylation or hypomethylation of genes in malignancy from ovarian carcinoma is not known in the art. There is nothing in the art to indicate that the genes in the above table are capable of being used as susceptible or prognostic markers and distinguishing between benign and malignant tumors.

According to the invention, the change of DNA methylation of one or more of the genes in the above table indicates that a subject is susceptible of ovarian neoplasms.

Among the genes in the above table, DNA hypermethylation of one or more of NPTX2, TNNI1, POU4F2, HS3ST2, CACNB2, TBX20, OR2L13, IGSF21, CD248, ADRA1A, NEFH, BNIP3, C1QTNF3, KCNA6, CEACAM4, CRNN, HFE2, TWIST1, GATA4, ATG4A, HIST1H2BN, THRB and MGST2, as compared to DNA methylation observed in non-cancer cells, indicates a poor prognosis in ovarian cancer. Preferably, the gene with DNA hypermethylation is ATG4A, HIST1H2BN, ADRA1A, CACNB2, GATA4, KCNA6, POU4F2, HS3ST2 or NEFH or any combination thereof. More preferably, the gene with DNA hypermethylation is ATG4A, HIST1H2BN, CEACAM4, GATA4 or IGSF21 or any combination thereof. More preferably, the gene with DNA hypermethylation is POU4F2, NEFH, HS3ST2 or any combination thereof. More preferably, the gene with DNA hypermethylation is CEACAM4, GATA4 or IGSF21 or any combination thereof. Alternatively, DNA hypomethylation of one or more of CACYBP, HIST1H2AJ, C1orf158, A4GALT, MLN, HIST1H3C, STC2 and ENG, as compared to DNA methylation observed in non-cancer cells, indicates a poor prognosis in ovarian cancer or a malignant ovarian cancer. Preferably, the gene with DNA hypomethylation is CACYBP or C1orf158 or any combination thereof. In the embodiments of the invention, the preferred gene with DNA hypermethylation for indicating poor prognosis in ovarian cancer or a malignant ovarian cancer is ATG4A, HIST1H2BN, CEACAM4, GATA4, NPTX2, TNNI1, POU4F2, HS3ST2, CACNB2, TBX20, OR2L13, IGSF21, CD248, ADRA1A, NEFH, BNIP3, C1QTNF3 or KCNA6 or any combination thereof. More preferably, the gene with DNA hypermethylation is ATG4A, HIST1H2BN, ADRA1A, CACNB2, GATA4, KCNA6, POU4F2, HS3ST2 or NEFH or any combination thereof. More preferably, the gene with DNA hypermethylation is ATG4A, HIST1H2BN, CEACAM4, GATA4 or IGSF21 or any combination thereof. More preferably, the gene with DNA hypermethylation is POU4F2, NEFH, HS3ST2 or any combination thereof. More preferably, the gene with DNA hypermethylation is CEACAM4, GATA4 or IGSF21 or any combination thereof. The preferred gene with DNA hypomethylation for indicating a poor prognosis in ovarian cancer or a malignant ovarian cancer is CACYBP or C1orf158 or any combination thereof. The preferred gene with DNA hypomethylation for indicating a poor prognosis in ovarian cancer or a malignant ovarian cancer is CACYBP, or MLN or a combination thereof.

The biomarker genes as set forth in above table encompass not only the particular sequences found in the publicly available database entries, but also variants of these sequences, including allelic variants. Variant sequences have at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to sequences in the database entries. Computer programs for determining percent identity are available in the art, including the Basic Local Alignment Search Tool (BLAST) available from the National Center for Biotechnology Information.

Conventional methods for DNA methylation detection use methylation specific and/or methylation sensitive restriction enzymes for restriction landmark analysis. Several advanced methods have been developed for DNA methylation detection, including bisulfite sequencing, methylation-specific PCR, MethyLight, microarray, field effect transistor (FET) based electronic charge detectors. Methods for detecting methylation status have been described in, for example U.S. Pat. Nos. 6,214,556, 5,786,146, 6,017,704, 6,265,171, 6,200,756, 6,251,594, 5,912,147, 6,331,393, 6,605,432, and 6,300,071 and US Patent Application publication Nos. 20030148327, 20030148326, 20030143606, 20030082609 and 20050009059, all of which are incorporated herein by reference. Other array based methods of methylation analysis are disclosed in U.S. patent application Ser. No. 11/058,566 (Pg Pub 20050196792 A1) and Ser. No. 11/213,273 (PgPub 20060292585 A1), which are both incorporated herein by reference in their entirety. For a review of some methylation detection methods, see, Oakeley, E. J., Pharmacology & Therapeutics 84:389-400 (1999). Available methods include, but are not limited to: reverse-phase HPLC, thin-layer chromatography, SssI methyltransferases with incorporation of labeled methyl groups, the chloracetaldehyde reaction, differentially sensitive restriction enzymes, hydrazine or permanganate treatment (m5C is cleaved by permanganate treatment but not by hydrazine treatment), sodium bisulfite, combined bisulphate-restriction analysis, methylation sensitive single nucleotide primer extension, methylation Specific polymerase chain reaction (MSP), CpG island microarrays and Infinium methylation assay.

In another aspect, the invention provides a method of making a treatment decision for a subject with ovarian cancer, comprising administering an effective amount of a demethylating agent to the subject, wherein the subject exhibits DNA hypermethylation of one or more of NPTX2, TNNI1, POU4F2, HS3ST2, CACNB2, TBX20, OR2L13, IGSF21, CD248, ADRA1A, NEFH, BNIP3, C1QTNF3, KCNA6, CEACAM4, CRNN, HFE2, TWIST1, GATA4, ATG4A, HIDT1H2BN, THRB and MGST2, or a polynucleotide sequence with at least 80% similarity thereof, as compared to DNA methylation observed in non-cancer cells. Preferably, the gene with DNA hypermethylation is ATG4A, HIST1H2BN, ADRA1A, CACNB2, GATA4, KCNA6, POU4F2, HS3ST2 or NEFH or any combination thereof. More preferably, the gene with DNA hypermethylation is ATG4A, HIST1H2BN, CEACAM4, GATA4 or IGSF21 or any combination thereof. More preferably, the gene with DNA hypermethylation is POU4F2, NEFH, HS3ST2 or any combination thereof. More preferably, the gene with DNA hypermethylation is CEACAM4, GATA4 or IGSF21 or any combination thereof.

According to the invention, suitable demethylating agents include, but are not limited to 5-aza-2′-deoxycytidine, 5-aza-cytidine, Zebularine, procaine, and L-ethionine.

In a further aspect, the invention provides a method of determining a therapeutic regimen for a subject having a poor prognosis or malignancy in ovarian cancer, comprising providing a chemotherapy to the subject, wherein the subject has DNA hypermethylation of one or more of NPTX2, TNNI1, POU4F2, HS3ST2, CACNB2, TBX20, OR2L13, IGSF21, CD248, ADRA1A, NEFH, BNIP3, C1QTNF3, KCNA6, CEACAM4, CRNN, HFE2, TWIST1, GATA4, ATG4A, HIST1H2BN, THRB and MGST2, or a polynucleotide sequence with at least 80% similarity thereof, as compared to DNA methylation observed in non-cancer cells, and/or DNA hypomethylation of one or more of CACYBP, HIST1H2AJ, C1orf158, A4GALT, MLN, HIST1H3C, STC2 and ENG, as compared to DNA methylation observed in non-cancer cells. Preferably, the gene with DNA hypermethylation is ATG4A, HIST1H2BN, ADRA1A, CACNB2, GATA4, KCNA6, POU4F2, HS3ST2 or NEFH or any combination thereof. More preferably, the gene with DNA hypermethylation is ATG4A, HIST1H2BN, CEACAM4, GATA4 or IGSF21 or any combination thereof. More preferably, the gene with DNA hypermethylation is POU4F2, NEFH, HS3ST2 or any combination thereof. More preferably, the gene with DNA hypermethylation is CEACAM4, GATA4 or IGSF21 or any combination thereof. Preferably, the gene with DNA hypomethylation is CACYBP or C1orf158 or any combination thereof. More preferably, the gene with DNA hypomethylation is CACYBP, or MLN or a combination thereof.

According to the invention, the method may further comprises making a treatment decision for a subject with ovarian cancer, such as to give chemotherapy to a subject having a poor prognosis, or to not give chemotherapy to a subject having a favorable prognosis. The method may further comprise treating said subject with adjuvant chemotherapy.

In another further aspect, the invention provides a kit for predicting risk or susceptibility of ovarian neoplasms or a prognosis or malignancy of ovarian cancer or making a treatment decision for a subject with ovarian cancer. The kit is assemblage of reagents for testing methylation. It is typically in a package which contains all elements, optionally including instructions. The package may be divided so that components are not mixed until desired. Components may be in different physical states. For example, some components may be lyophilized and some in aqueous solution. Some may be frozen. Individual components may be separately packaged within the kit. The kit may contain reagents, as described above for differentiating methylated and non-methylated cytosine residues. Desirably the kit will contain oligonucleotide primers which specifically hybridize to regions within the transcription start sites of the genes identified by the invention. Typically the kit will contain both a forward and a reverse primer for a single gene. Specific hybridization typically is accomplished by a primer having at least 12, 14, 16, 18, or 20 contiguous nucleotides which are complementary to the target template. Often the primer will be 100% identical to the target template. If there is a sufficient region of complementarity, e.g., 12, 15, 18, or 20 nucleotides, then the primer may also contain additional nucleotide residues that do not interfere with hybridization but may be useful for other manipulations. Examples of such other residues may be sites for restriction endonuclease cleavage, for ligand binding or for factor binding or linkers. The oligonucleotide primers may or may not be such that they are specific for modified methylated residues. The kit may optionally contain oligonucleotide probes. The probes may be specific for sequences containing modified methylated residues or for sequences containing non-methylated residues. Like the primers described above, specific hybridization is accomplished by having a sufficient region of complementarity to the target. The kit may optionally contain reagents for modifying methylated cytosine residues. The kit may also contain components for performing amplification, such as a DNA polymerase and deoxyribonucleotides. Means of detection may also be provided in the kit, including detectable labels on primers or probes. Kits may also contain reagents for detecting gene expression for one of the markers of the present invention. Such reagents may include probes, primers, or antibodies, for example. In the case of enzymes or ligands, substrates or binding partners may be sued to assess the presence of the marker.

The materials for use in the methods of the present invention are suited for preparation of kits produced in accordance with well known procedures. The invention thus provides kits comprising agents, which may include gene-specific or gene-selective probes and/or primers, for quantitating the expression of the disclosed genes for predicting prognostic outcome or malignant level. Such kits may optionally contain reagents for the extraction of RNA from tumor samples, in particular fixed paraffin-embedded tissue samples and/or reagents for RNA amplification. In addition, the kits may optionally comprise the reagent(s) with an identifying description or label or instructions relating to their use in the methods of the present invention. The kits may comprise containers (including microtiter plates suitable for use in an automated implementation of the method), each with one or more of the various reagents (typically in concentrated form) utilized in the methods, including, for example, pre-fabricated microarrays, buffers, the appropriate nucleotide triphosphates (e.g., dATP, dCTP, dGTP and dTTP; or rATP, rCTP, rGTP and UTP), reverse transcriptase, DNA polymerase, RNA polymerase, and one or more probes and primers of the present invention (e.g., appropriate length poly(T) or random primers linked to a promoter reactive with the RNA polymerase). Mathematical algorithms used to estimate or quantify prognostic or predictive information are also properly potential components of kits.

All publications and patent documents cited in this application are incorporated by reference in their entirety for all purposes to the same extent as if each individual publication or patent document were so denoted. By their citation of various references in this document, Applicants do not admit any particular reference is “prior art” to their invention.

### EXAMPLE

### Example 1

### Identification of 25 Biomarker Genes of the Invention

The example is to discover novel DNA methylation biomarkers for ovarian cancer prognosis prediction and screening. Tissue samples were collected with the informed consent of patients at the Tri-Service General Hospital, National Defense Medical Center, Taipei, Taiwan. This study was approved by the Institutional Review Board. 61 independence patients' ovarian samples that included 49 malignant and 12 benign tissues were used. These samples were obtained during surgery and were frozen immediately in liquid nitrogen and stored at −80° C. until analysis. The presence of malignant cells was confirmed by the histological examination. Gynecologic pathologists reviewed all of the specimens for assessing histology. Progression free survival (PFS) was defined as the time from first operates to progressive disease. Patients presented persistent disease after the first line standard treatment were excluded for PFS analysis. Overall survival (OS) was defined as the time from first operates to death due to EOC.

Genomic DNA was extracted from tissue samples using a commercial DNA extraction kit (QIAmp Tissue Kit; Qiagen, Hilden, Germany). Genomic serum DNA was extracted from 1 ml of serum using a commercial DNA blood mini-kit (QIAmp DNA Blood Mini Kit; Qiagen) according to the protocol described in the user manual.

Of the genomic DNA, 1 μg was bisulfite modified using the CpGenome Fast DNA Modification Kit (Chemicon-Millipore, Bedford, Mass., USA) according to the manufacturer's recommendations and redissolved in 70 ml nuclease-free water. We compared the promoter methylation status in patients with epithelial ovarian cancer, benign and normal ovarian tissues using Bisulfite modification, quantitative methylation-specific PCR (QMSP) and validated with pyrosequencing analysis. QMSP was performed in a TaqMan probe system using the LightCycler 480 Real-Time PCR System (Roche, Indianapolis, Ind., USA). The DNA methylation level estimated for the methylation index (M-index), with the formula: 10,000×2^{[(Cp of COL2A)-(Cp of Gene)]}. Test results with Cp values for COL2A greater than 36 were defined as detection failure. The primers for pyrosequencing were designed by PyroMark Assay Design 2.0 software (Qiagen) to amplify and sequencing bisulfite-treated DNA. The universal and amplification primers are obtained according to previous publication. The biotinylated PCR product was bound to streptavidin sepharose beads, washed, and denatured. After addition sequencing primer to single-stranded PCR products, the pyrosequencing was carried through by PyroMark Q24 software (Qiagen, German) according to the manufacturer's instructions.

Infinium Methylation Assay was used to analyze the methylation profile of every clinical sample (Laurent L., Wong E., Li G, Huynh T, Tsirigos A., et al., 2010, “Dynamic changes in the human methylome during differentiation,” Genome Res 20: 320-331). Differential methylation analysis comparing the methylation profiles of patients with different survival outcomes was conducted to select candidate genes (Pavlidis P, Noble W S, 001, “Analysis of strain and regional variation in gene expression in mouse brain,” Genome Biol 2: RESEARCH0042). A systematic method shown in below scheme to verify methylation DNA in pools ovarian carcinoma mad cell lines. Each patient's samples were verified in an ovarian cohort.

We evaluated the extreme discrimination of cutoff value for methylation status of each gene to distinguish recurrence and non-recurrence patients by calculating the area under the receiver operating characteristic (ROC) curve (AUC). We used the same strategy to estimate the optimal cutoff value to distinguish death and survival patients. According to the optimal cutoff value from AUC analysis, we defined the all methylation value to be high and low binomial codes to do further statistics. The correlation between categorical variables of different groups was determined using chi-square test, Fisher's exact test or Mann-Whitney U test. PFS and OS described the survival function for Kaplan-Meier survival analysis, univariate and multivariate COX regression analysis. A univariate COX regression analysis was calculate Hazard ratios (HR) and 95% confidence interval (CI) for the evaluation of clinicopathological characteristics risk for each candidate gene. The medium survival times were calculated for patients with high vs. low methylation in candidate genes via log-rank test. The multivariate Cox proportional hazards model was performed to determine the independent prognostic value of age, DNA methylation status, stage, grade, and histology subtype. The whole statistics were considered the two-sided test and p-value less than 0.05 as significant. All statistical calculations were primarily performed using the statistical package SPSS version 17.0 for windows (SPSS, Inc., Chicago, Ill.).

Twenty five genes having statistic significance and large differential methylation between short and long survivals were detected. Table 1 shows the summary of polymerase chain reaction and bisulfite pyrosequencing primers. Table 2 shows univariate COX regression analysis of overall survival in 25 genes. Table 3 shows differential methylation levels between benign and malignant tumors. Table 4 shows multivariat analysis of methylation and clinicopathological factors for progression free survival (PFS) and overall survival (OS).

TABLE 1

| Primer | Forward Primer Sequence | Reward Primer Sequence |
|---|---|---|
| Name | (5′ - 3′) | (5′ - 3′) |
| ADRA1A | CTTAGTCATGCCCATTGGGTC | CTGCAGAGACACTGGATTCTC |
| | (SEQ ID NO: 32) | (SEQ ID NO: 47) |
| BNIP3 | TGGACGGAGTAGCTCCAAGAG | CCGACTTGACCAATCCCATATC |
| | (SEQ ID NO: 33) | (SEQ ID NO: 48) |
| C1orf158 | GACAAGACACCCCAATCCATT | TGTTTGTAAGGTAGCCCCTCAA |
| | (SEQ ID NO: 34) | (SEQ ID NO: 49) |
| CACNB2 | CTATCTGGAGGCCTACTGGAAG | TCAGTCCTCTGATCACCTTGAG |
| | (SEQ ID NO: 35) | (SEQ ID NO: 50) |
| CACYBP | TCTCTGTGGAAGGCAGTTCAA | TCTGTTTCAGTGTCATAGGAGGG |
| | (SEQ ID NO: 36) | (SEQ ID NO: 51) |
| CEACAM4 | CAGTTACGACTCTGACCAAGCAAC | CTTCCAGTCCTGGAGAGAAGCAG |
| | (SEQ ID NO: 37) | (SEQ ID NO: 52) |
| HFE2 | TCCTCTTTGTCCAAGCCACCAG | CATCTTCAAAGGCTACAGGAAG |
| | (SEQ ID NO: 38) | (SEQ ID NO: 53) |
| HIST1H3C | GCAGCTTGCTACTAAAGCAGC | CGCACAGATTGGTGTCTTCG |
| | (SEQ ID NO: 39) | (SEQ ID NO: 54) |
| HS3ST2 | GCCGTGCTGGAGTTTATCC | GGAGCCTCTTGAGTGACAAAG |
| | (SEQ ID NO: 40) | (SEQ ID NO: 55) |
| IGSF21 | TTCCTCAACGTCATGGCTCC | CCTCCAGACACGATGCAGAC |
| | (SEQ ID NO: 41) | (SEQ ID NO: 56) |
| KCNA6- | GTTACAATGACCACGGTAGGTT | GTCCGTTGTCAGTTGCCCTC |
| 1252F/1467R | (SEQ ID NO: 42) | (SEQ ID NO: 57) |
| MLN | ATGGTATCCCGTAAGGCTGTG | CTGGAGTTCGCCATAGGTGAA |
| | (SEQ ID NO: 43) | (SEQ ID NO: 58) |
| NEFH | CGAGGAGTGGTTCCGAGTG | GCATAGCGTCTGTGTTCACCT |
| | (SEQ ID NO: 44) | (SEQ ID NO: 59) |
| POU4F2-78F/299R | CTCGGCACTGCACAGCACCT | ACTCTCATCCAGCCCGCCGA |
| | (SEQ ID NO: 45) | (SEQ ID NO: 60) |
| TWIST1 | ACTTCCTCTACCAGGTCCTCCAGAG | ACAATGACATCTAGGTCTCCGGCCC |
| | (SEQ ID NO: 46) | (SEQ ID NO: 61) |

Bisulfited Pyrosequencing PCR

| | | |
|---|---|---|
| ADRA1A_py06 | TTTAGGTGGGGTAGTTTAAAATGTAGGTA | CCTTACAACATACAATTCCAAAATTAC |
| | (SEQ ID NO: 62) | (SEQ ID NO: 84) |
| BNIP3_py03 | TGGGAGAGGGGTAGAGGT | CCTCAATTTCCCCACTAAC |
| | (SEQ ID NO: 63) | (SEQ ID NO: 85) |
| BNIP3_py05 | TGGGAGAGGGGTAGAGGT | ATCCCACCCCCCCTTCAAAAA |
| | (SEQ ID NO: 64) | (SEQ ID NO: 86) |
| BNIP3_py07 | GGGTTGAGGGATGTGTTTTAGT | ACCCCAAACCTCTACCCCT |
| | (SEQ ID NO: 65) | (SEQ ID NO: 87) |
| C1orf158_py04 | GGAGGATGAGGTAGGAGAATG | AAAACTCCAAAAAACTATATATTCCATCTT |
| | (SEQ ID NO: 66) | (SEQ ID NO: 88) |
| CACNB2_py04, 05, 06 | GTTGTGGGAGGAGATTTGGATATG | ACCCCCCTAAAAACTCCCCTCTC |
| | (SEQ ID NO: 67) | (SEQ ID NO: 89) |
| CACYBP_03, 04 | AGGAGAAAAATGGGGAGGAGT | CCCTTTTATTAAAACCTTAACCTAAACT |
| | (SEQ ID NO: 68) | (SEQ ID NO: 90) |
| CD248_py02 | GGGTAAGAAAGGAGTGGGTATG | CCAAACCCCATAAAACTAAAAATCA |
| | (SEQ ID NO: 69) | (SEQ ID NO: 91) |
| CD248_py03, 04 | TTTTAGGGGAAGAGGGAGTAGGG | CAACAACCCAAAAATCCTAACCCAATAT |
| | (SEQ ID NO: 70) | (SEQ ID NO: 92) |
| HS3ST2_py02, 03, 04 | AGGGGGAGGGTTAGGTTTT | ATTACATTTCCAACATCTCCC |
| | (SEQ ID NO: 71) | (SEQ ID NO: 93) |
| HS3ST2_py06 | AGGATAGGGAGATGTTGGAAATGT | ACCCAAAACCCTATAAACCAT |
| | (SEQ ID NO: 72) | (SEQ ID NO: 94) |
| IGSF21_py01 | ATGAGGGTATTTATAGTTGGTAAGGTTAGA | CCCCTCACTCAAAACTAACTT |
| | (SEQ ID NO: 73) | (SEQ ID NO: 95) |
| IGSF21_py02 | AAGAAGTTGGAGGTAGTAAGTTAGT | CCCCCCCCCTCCTTACCCT |
| | (SEQ ID NO: 74) | (SEQ ID NO: 96) |
| KCNA6_py01 | GGGAAAGGTATTGATTGATTTGTTA | TACCAACCTCTCCAATATCTACAA |
| | (SEQ ID NO: 75) | (SEQ ID NO: 97) |
| MLN_py02 | GTTTTAGGGGGAAGATTGAAGAGAA | ACCCATTAACCTTTAACCACAACT |
| | (SEQ ID NO: 76) | (SEQ ID NO: 98) |
| MLN_py07 | TTTAGGGTTGGGAGGTATATAAGA | CACCCACAACAACCTCTACTTTAC |
| | (SEQ ID NO: 77) | (SEQ ID NO: 99) |
| NEFH_py05 | GTGAGAGGGTGGGGAGGA | CATCCTACCCCTATTCCCATCAA |
| | (SEQ ID NO: 78) | (SEQ ID NO: 100) |
| NEFH_py07 | GAGTGGAAGTAGTTGGAGGAGTTA | ACCCTCTCACTACCAAAAAATTAAAC |
| | (SEQ ID NO: 79) | (SEQ ID NO: 101) |
| OR2L13_py05 | AGGGTTATTTGTAATGTGGGTAAG | CAAAAATTTTCCTACCCAAAAACT |
| | (SEQ ID NO: 80) | (SEQ ID NO: 102) |
| POU4F2_py06, 07 | GTTGGAGGTTGGTTTTTAGGTAGG | CTACTCCCCTCAAACTTAAATCCT |
| | (SEQ ID NO: 81) | (SEQ ID NO: 103) |
| TBX20_py05, 07 | GGTGGGGAATAGAGGTTAGT | AACCCAACTTACCCAAAAATT |
| | (SEQ ID NO: 82) | (SEQ ID NO: 104) |
| TWIST1_py04 | TGGGAGAGATGAGATATTATTTATTGTGT | TCTAACAATTCCTCCTCCCAAACCATTCA |
| | (SEQ ID NO: 83) | (SEQ ID NO: 105) |

TABLE 2

| | | | | |
|---|---|---|---|---|
| Gene | GeneID | HR | 95% CI | P^{a} |

| | | | | | |
|---|---|---|---|---|---|
| KCNA6 | Gene_22 | 15.16 | 3.54 | 64.98 | 0.000 |
| POU4F2 | Gene_13 | 8.69 | 2.14 | 35.32 | 0.003 |
| HFE2 | Gene_24 | 8.29 | 2.12 | 32.40 | 0.002 |
| GATA4 | Gene_2 | 7.64 | 1.54 | 37.81 | 0.013 |
| ADRA1A | Gene_20 | 6.93 | 1.77 | 27.07 | 0.005 |
| HS3ST2 | Gene_16 | 6.90 | 1.79 | 26.62 | 0.005 |
| TBX20 | Gene_6 | 6.38 | 1.67 | 24.42 | 0.007 |
| CRNN | Gene_17 | 5.27 | 0.67 | 41.38 | 0.114 |
| NPTX2 | Gene_5 | 4.28 | 0.92 | 20.03 | 0.085 |
| CACN82 | Gene_23 | 4.25 | 1.13 | 15.94 | 0.032 |
| BNIP3 | Gene_25 | 4.02 | 1.06 | 15.20 | 0.040 |
| TNNI1 | Gene_12 | 3.55 | 0.72 | 17.40 | 0.118 |
| CD248 | Gene_4 | 3.19 | 0.66 | 15.53 | 0.150 |
| C1QTNF3 | Gene_9 | 2.96 | 0.75 | 11.65 | 0.121 |
| NEFH | Gene_7 | 2.38 | 0.69 | 8.21 | 0.171 |
| IGSF21 | Gene_3 | 2.24 | 0.60 | 8.38 | 0.233 |
| CEACAM4 | Gene_1 | 2.09 | 0.26 | 17.07 | 0.492 |
| OR2L13 | Gene_19 | 1.95 | 0.49 | 7.82 | 0.345 |
| TWIST1 | Gene_10 | 1.39 | 0.29 | 6.71 | 0.681 |
| MLN | Gene_18 | 0.63 | 0.17 | 2.35 | 0.490 |
| HIST1H2AJ | Gene_8 | 0.37 | 0.09 | 1.50 | 0.165 |
| A4GALT | Gene_11 | 0.28 | 0.05 | 1.31 | 0.102 |
| C1orf158 | Gene_15 | 0.22 | 0.06 | 0.84 | 0.026 |
| HIST1H3C | Gene_21 | 0.10 | 0.01 | 0.83 | 0.033 |
| CACYBP | Gene_14 | 0.08 | 0.02 | 0.34 | 0.001 |

Abbreviations:
HR, Hazard ratio;
CI, confidence interval
^{a}Cox regression test; Statistic significant is p < .05

TABLE 3

Mean of
methylation level ± SD

| Gene | Benign | Malignant | P-value^{a} |
|---|---|---|---|
| ADRA1A | 0.11 ± 0.05 | 0.31 ± 0.21 | <0.000 |
| CACNB2 | 0.04 ± 0.03 | 0.23 ± 0.29 | <0.000 |
| GATA4 | 0.14 ± 0.05 | 0.36 ± 0.21 | <0.000 |
| KCNA6 | 0.17 ± 0.04 | 0.32 ± 0.25 | <0.000 |
| NEFH | 0.17 ± 0.12 | 0.35 ± 0.21 | =0.005 |
| NPTX2 | 0.26 ± 0.14 | 0.49 ± 0.25 | <0.000 |
| TBX20 | 0.06 ± 0.04 | 0.28 ± 0.25 | <0.000 |

^{a}The statistic significant is <0.05 using 2-tails of T-TEST

TABLE 4

| | | POU4F2 | | | | NEFH | | | | HS3ST2 | | | |
|---|---|---|---|---|---|---|---|---|---|---|---|---|---|
| | Category | HR | 95% CI | | P | HR | 95% CI | | P | HR | 95% CI | | P |
| OS | Mehtylation | 7.24 | 3.36 | 15.61 | <0.001 | 2.73 | 1.43 | 5.21 | 0.002 | 3.07 | 1.56 | 6.04 | 0.001 |
| | Age | 1.03 | 1.01 | 1.06 | 0.017 | — | | | 0.094 | — | | | 0.266 |
| | FIGO Stage | 35.51 | 4.43 | 284.83 | 0.001 | 18.09 | 2.39 | 136.82 | 0.005 | 13.16 | 1.70 | 102.08 | 0.014 |
| | Grading | 3.52 | 1.17 | 10.53 | 0.025 | 3.68 | 1.27 | 10.65 | 0.016 | 3.07 | 1.56 | 6.04 | 0.001 |
| PFS | Mehtylation | — | | | 0.638 | 2.33 | 1.19 | 4.57 | 0.014 | 3.96 | 1.75 | 8.95 | 0.001 |
| | FIGO Stage | 9.97 | 3.47 | 28.62 | <0.001 | 9.49 | 3.30 | 27.29 | <0.001 | 11.62 | 3.99 | 33.81 | <0.001 |
| | Grading | — | | | 0.153 | — | | | 0.113 | — | | | 0.127 |
| | Histopathology | — | | | 0.825 | — | | | 0.992 | — | | | 0.605 |

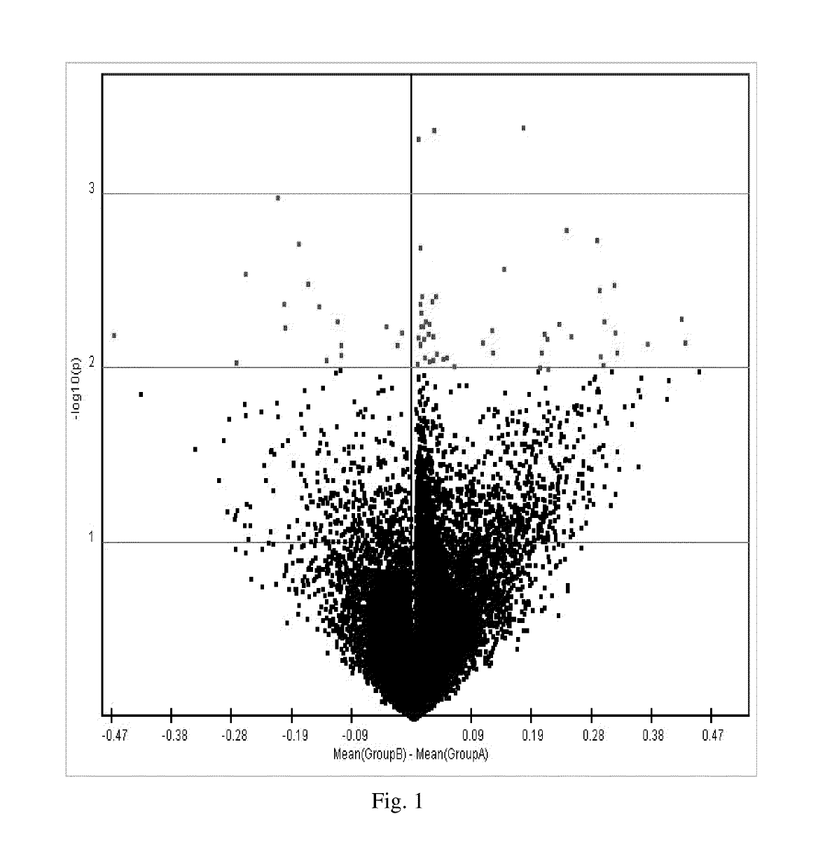

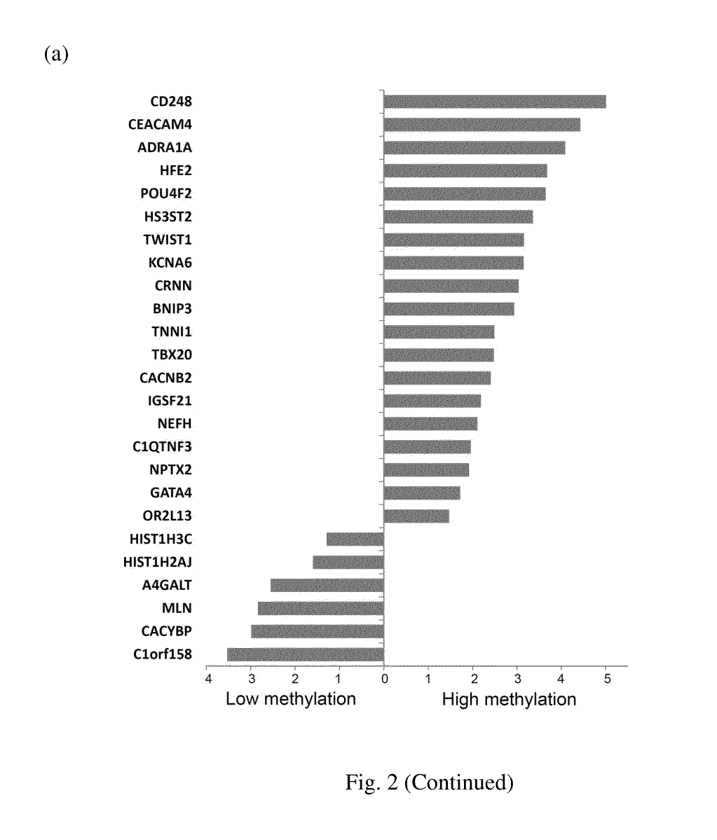

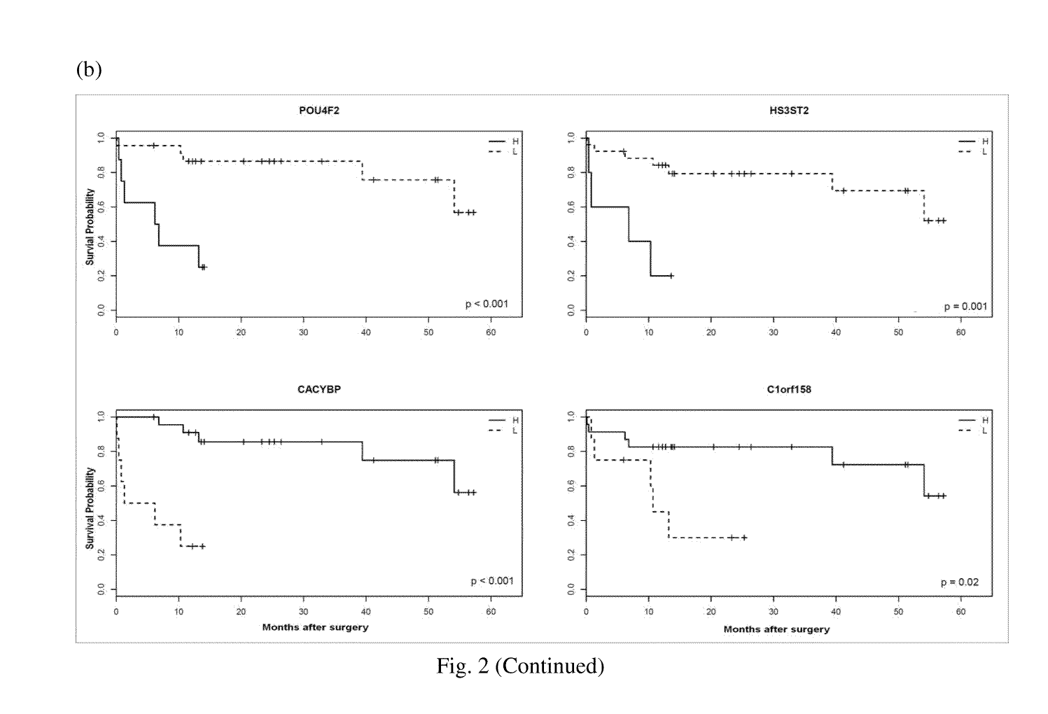

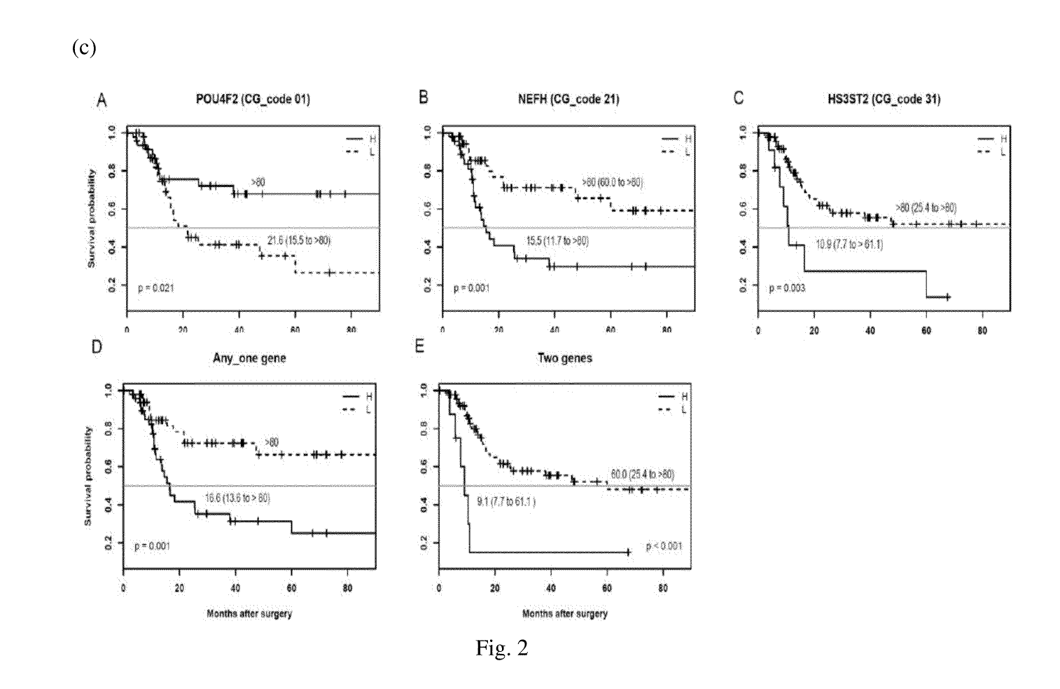

FIG. 1 shows differential methylation analysis of patients with different prognosis (long and short survival). The patients were divided into two groups at the survival of 3 years. As shown in FIG. 1, the dots at first second blocks reveal the differentially methylated (right) or unmethylated (left) genes. The dots that are the most significant are selected candidate genes for further evaluation. FIG. 2 shows correlation of DNA methylation of candidate genes with survival. The results show that 19 genes have high risk in hypermethylation status, and the other 6 genes have higher risk in hypomethylation. As shown in FIG. 2 a), DNA hypermethylation with poor prognosis are list at right side. DNA hypomethylation with poor prognosis are listed at the left side. FIG. 2b) shows Kaplan-meier survival estimates of overall survival (OS) in patients with ovarian carcinoma. For POU4F2 and HS3ST2, patients are grounded into high methylation (H) and low methylation (L) according to 0.4 AVG values, and high methylation patients exhibit short survival time. For CACYBP and C1orf158, patients are grounded into high methylation (H) and low methylation (L) according to 0.4 AVG values, and low methylation patients exhibit short survival time. FIG. 2 c) shows Kaplan-meier survival estimates of the progression-free survival (PFS) in patients with ovarian carcinoma. High methylation of NEFH and HS3ST2 are risk factors, whilst low methylation of POU4F2 is risk factor. Patients with any risk factor of these methylation statues (patient may have one, two or three risk factors) will have poor prognosis as shown at the left. Patients without any risk factors of these methylation statues will have better prognosis as shown at the right. Patients with any two of the three risk factors (patients may have two or three risk factors) will have poor prognosis as shown at the left. Patients without any risk factors or with only one risk factor have better prognosis.

### Example 2

### Identification of 6 Biomarker Genes of the Invention

Tissue samples were collected with the informed consent of patients at the Tri-Service General Hospital, National Defense Medical Center, Taipei, Taiwan. This study was approved by the Institutional Review Board. The patients included 110 with epithelial ovarian carcinomas (EOC), 60 with a benign ovarian tumor and 28 with normal ovarian tissue whose diagnosis included histological subtype and grade. These samples were obtained during surgery and were frozen immediately in liquid nitrogen and stored at −80° C. until analysis. The presence of malignant cells was confirmed by the histological examination. Gynecologic pathologists reviewed all of the specimens for assessing histology. Progression free survival (PFS) was defined as the time from first operates to progressive disease. Patients presented persistent disease after the first line standard treatment were excluded for PFS analysis. Overall survival (OS) was defined as the time from first operates to death due to EOC.

The genomic DNA extraction, QMSP, Infinium methylation assay, Differential methylation analysis and Kaplan-Meier survival analysis were performed as stated in Example 1. Six genes having statistic significance and large differential methylation between short and long survivals were detected. The bisulfite pyrosequencing primers are shown in Table 5.

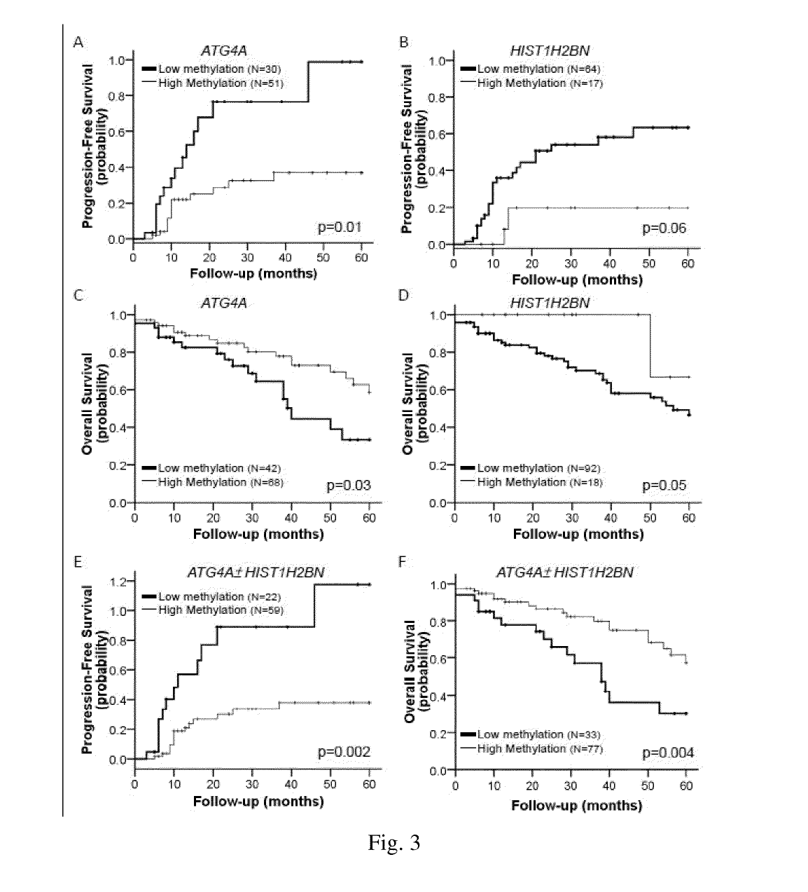

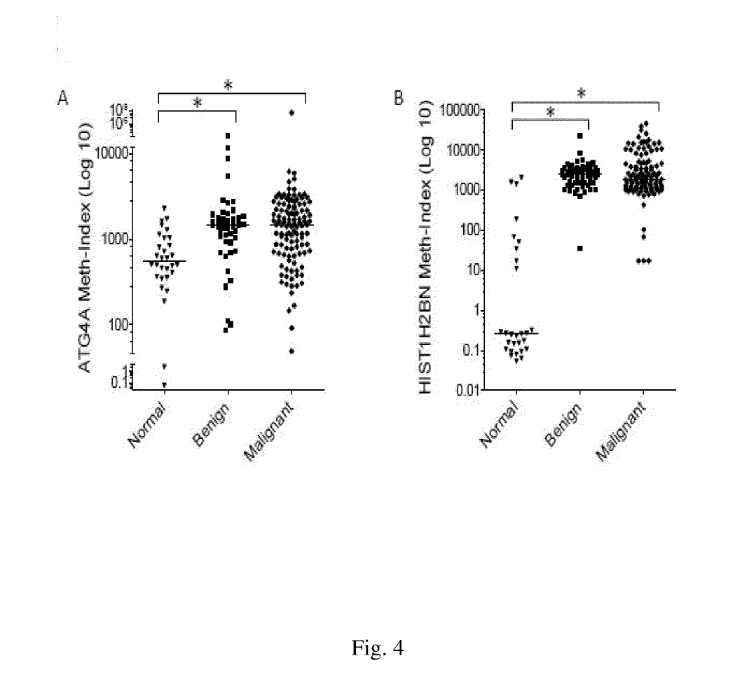

The prognostic significance of these DNA methylations was tested. The results of the univariate Cox regression analysis for progression-free survival (PFS) and overall survival (OS) are presented in Table 7. As expected, FIGO stage and histological grades, were associated with PFS and OS. ATG4A low methylation was significantly associated with PFS (HR=2.50; 95% CI 1.18-5.26) and OS (HR=2.09; 95% CI 1.08-4.04). A borderline significant correlation between the presence of methylation of HIST1H2BN and recurrence was observed. The prognosis of patients with low methylation of HIST1H2BN was slightly associated with a worse survival; the HR values were 6.08 (95% CI, 0.83-44.45). The Kaplan-Meier analysis for the PFS and OS of cancer patients revealed that patients with low methylation of ATG4A or HIST1H2BN conferred significantly shorter PFS (FIGS. 3A and 3B; P=0.01 and 0.06, respectively) and more likely to die (FIGS. 3C and 3D; P=0.03 and 0.05, respectively) within the follow-up period than patients with high methylation. The patients with cisplatin resistance were significantly associated with low methylation of ATG4A (Table 6). In the multivariate Cox proportional hazards regression analysis, after adjusting for the related factors, methylation of HIST1H2BN showed an independent effect on PFS and OS (Table 7). Patients with low methylation of HIST1H2BN had a hazard ratio of 5.16 (95% CI, 1.22-21.94) for PFS and 8.08 (95% CI, 1.10-59.37) for OS. Although the low methylation of ATG4A was a significant predictor of death in the univariate analysis, this effect was no longer evident in the multivariate analysis. Furthermore, we take ATG4A and HIST1H2BN together to define the low methylation group as both genes are low methylated, and high methylation group as the others. There shows the good discrimination between the low and high methylation groups cancer patients of PFS and OS in FIGS. 3E and 3F (log-rank P=0.002 and 0.004, respectively).

The methylation status of ATG4A and HIST1H2BN were further validated in clinical materials including normal ovarian tissues, benign and malignant tumor tissues using qMSP (FIGS. 3A and 3B). Both benign and malignant tumors confer significantly higher methylation level than normal ovarian tissues.

TABLE 5
QMSP primer

| | Forward primer sequence | Reverse primer sequence |
|---|---|---|
| HIST1H2BN | TTCGGGGGTGGGAGAGAGC | ACAAAAAACATACACACACGCACG |
| | (SEQ ID NO: 106) | (SEQ ID NO: 112) |
| ATG4A | GGGGTTTTCGTTAGGGTC | CTAAATCTCTCCGCAATCG |
| | (SEQ ID NO: 107) | (SEQ ID NO: 113) |
| THRB | ACGGGTCGGGTCGGTC | CACCCACCCGATTACCTACG |
| | (SEQ ID NO: 108) | (SEQ ID NO: 114) |
| STC2 | CGGGAAAGGAAAGTTTTGGAAGT | ACGAAAAAACACGCGAACAAAT |
| | (SEQ ID NO: 109) | (SEQ ID NO: 115) |
| ENG | CGTTTGTTTTTTTCGGGTTTTC | CTAATCCGTACACCGAAAACCG |
| | (SEQ ID NO: 110) | (SEQ ID NO: 116) |
| MGST2 | AAGCGTTATTTATTTTTTCGTGC | CACGCGCACACACACGA |
| | (SEQ ID NO: 111) | (SEQ ID NO: 117) |

Pyrosequencing primer

| | Forward primer sequence | Reverse primer sequence |
|---|---|---|
| HIST1H2BN | AGTATTATATTTTAGGGGGTGGGAGA | ACAAACCAATTTAAAAAACAACTCT |
| | (SEQ ID NO: 118) | (SEQ ID NO: 124) |
| ATG4A | GGGAAAATATTTGAGGTTTGTGG | CCCTAACTACTAAAACTAACCAAATAA |
| | (SEQ ID NO: 119) | (SEQ ID NO: 125) |
| THRB | GGATTAGAGGAGGTTTTAAGAAGAG | CTCCCCACCTACCTCCCCAAATAT |
| | TTAG | (SEQ ID NO: 126) |
| | (SEQ ID NO: 120) | |
| STC2 | GGGAAAGGAAAGTTTTGGAAGT | AAATTTCATCACCCACTACC |
| | (SEQ ID NO: 121) | (SEQ ID NO: 127) |
| ENG | GGTAGTTATTTTAGAAGGTTGGAGTA | CCCTAAATCCCTAAACACCTACTTATA |
| | GG | (SEQ ID NO: 128) |
| | (SEQ ID NO: 122) | |
| MGST2 | GGTTGGAGGGTTGGTTTTA | ACACCAACTTCCCATACCTCTTACTTT |
| | (SEQ ID NO: 123) | (SEQ ID NO: 129) |

TABLE 6
Table 6. Patient characteristics and clinicopathological features by ATG4A and
HIST1H2BN methylation status

| | ATG4A | | | | | HIST1H2BN | | | | |
|---|---|---|---|---|---|---|---|---|---|---|
| | High methylation | | Low methylation | | | High methylation | | Low methylation | | |
| Characteristics | (N = 68; 61.8%) | | (N = 42; 38.2%) | | P value | (N = 18; 16.4%) | | (N = 92; 83.6%) | | P value |
| Age (years) | | | | | 0.71 | | | | | 0.16 |
| Mean, range | 54.1 | (19-90) | 53.0 | (18-79) | | 58.1 | (39-79) | 52.8 | (18-90) | |
| FIGO Stage | | | | | 0.002* | | | | | 0.49 |
| Early (I, II) | 33 | (48.5) | 8 | (19.0) | | 8 | (44.4) | 33 | (35.9) | |
| Late (III, | 35 | (51.5) | 34 | (81.0) | | 10 | (55.6) | 59 | (64.1) | |
| IV) | | | | | | | | | | |
| Grade^{a} | | | | | 0.16 | | | | | 0.59 |
| G1/G2 | 31 | (46.3) | 13 | (32.5) | | 6 | (35.3) | 38 | (42.2) | |
| G3 | 36 | (53.7) | 27 | (67.5) | | 11 | (64.7) | 52 | (57.8) | |
| Histology | | | | | 0.64 | | | | | 0.29 |
| Serous type | 44 | (64.7) | 29 | (69.0) | | 10 | (55.6) | 63 | (68.5) | |
| Other types | 24 | (35.3) | 13 | (31.0) | | 8 | (44.4) | 29 | (31.5) | |
| Platinum | | | | | 0.02* | | | | | 0.33 |
| Response | | | | | | | | | | |
| Sensitive | 50 | (98.0) | 25 | (83.3) | | 17 | (100) | 58 | (90.6) | |
| Resistant | 1 | (2.0) | 5 | (16.7) | | 0 | (0) | 6 | (9.4) | |

Abbreviations:
SD, standard deviation.
^{a}Grade data are missing in three patients.
*Significantly correlated with outcome, p < 0.05.

TABLE 7
Table 7. Univariate and Multivariate Cox regression analysis for progression-free survival and overall survival of ovarian cancer patients

| Event | Progression-Free Survival | | | Overall Survival | | |
|---|---|---|---|---|---|---|
| Variable | Crude HR (95% CI) | Adjusted HR (95% CI) | | Crude HR (95% CI) | Adjusted HR (95% CI) | |
| Age (years) | 1.02 (0.99, 1.05) | 1.01 (0.98, 1.04) | 1.01 (0.98, 1.04) | 1.03 (1.01, 1.05)* | 1.01 (0.99, 1.04) | 1.01 (0.99, 1.04) |
| ATG4A | | ^{a} | | | ^{c} | |
| High | 1.00 (reference) | 1.00 (reference) | | 1.00 (reference) | 1.00 (reference) | |
| methylation | | | | | | |
| Low | 2.50 (1.18, 5.26)* | 1.17 (0.54, 2.55) | | 2.09 (1.08, 4.04)* | 1.39 (0.70, 2.74) | |
| methylation | | | | | | |
| HIST1H2BN | | | ^{b} | | | ^{d} |
| High | 1.00 (reference) | | 1.00 (reference) | 1.00 (reference) | | 1.00 (reference) |
| methylation | | | | | | |
| Low | 3.39 (0.80, 14.32) | | 5.16 (1.22, 21.94)* | 6.08 (0.83, 44.45) | | 8.08 (1.10, 59.37)* |
| methylation | | | | | | |
| FIGO Stage | | | | | | |
| Early (I, II) | 1.00 (reference) | 1.00 (reference) | 1.00 (reference) | 1.00 (reference) | 1.00 (reference) | 1.00 (reference) |
| Late (III, IV) | 11.17 (3.36, 37.12)* | 8.06 (1.84, 35.30)* | 8.48 (2.00, 35.93)* | 15.72 (3.75, 65.83)* | 7.45 (1.62, 34.17)* | 8.23 (1.84, 36.76)* |
| Grade | | | | | | |
| G1/G2 | 1.00 (reference) | 1.00 (reference) | 1.00 (reference) | 1.00 (reference) | 1.00 (reference) | 1.00 (reference) |
| G3 | 4.07 (1.72, 9.65)* | 1.87 (0.74, 4.74) | 1.89 (0.75, 4.80) | 7.55 (2.65, 21.50)* | 3.07 (1.02, 9.29)* | 3.26 (1.08, 9.83)* |
| Histology | | | | | | |
| Serous type | 3.12 (1.08, 8.99)* | 0.84 (0.20, 3.61) | 0.84 (0.20, 3.57) | 1.40 (0.64, 3.07) | 0.39 (0.16, 0.96)* | 0.42 (0.17, 1.04) |
| Other types | 1.00 (reference) | 1.00 (reference) | 1.00 (reference) | 1.00 (reference) | 1.00 (reference) | 1.00 (reference) |

Abbreviations:
HR, hazard ratio;
CI, confidence interval.
^{a}The hazard ratio adjusted by gene methylation level, stage, grade and histology.
^{b}The hazard ratio adjusted by stage, grade and histology.
^{c}The hazard ratio adjusted by age, gene methylation level, stage and grade.
^{d}The hazard ratio adjusted by age, stage and grade.
*

What is claimed is:

1. A method of predicting risk or susceptibility of ovarian neoplasms or predicting prognosis or malignancy in a subject diagnosed with an ovarian neoplasm in a subject, comprising assessing DNA methylation of one or more of the following genes in an ovarian neoplasm sample obtained from said subject: NPTX2, TNNI1, POU4F2, 5 HS3ST2, CACNB2, TBX20, OR2L13, IGSF21, CD248, ADRA1A, NEFH, BNIP3, C1QTNF3, KCNA6, CEACAM4, CRNN, HFE2, TWIST1, GATA4, CACYBP, HIST1H2AJ, C1orf158, A4GALT, MLN, HIST1H3C, STC2, ATG4A, ENG, HIST1H2BN, MGST2 and THRB, or a polynucleotide sequence with at least 80% similarity thereof; wherein change of DNA methylation indicates that the subject is susceptible of ovarian neoplasms or a poor prognosis or a malignant ovarian cancer.

2. (canceled)

3. The method of claim 1, wherein DNA hypermethylation of one or more of NPTX2, TNNI1, POU4F2, HS3ST2, CACNB2, TBX20, OR2L13, IGSF21, CD248, ADRA1A, NEFH, BNIP3, C1QTNF3, KCNA6, CEACAM4, CRNN, HFE2, TWIST1, GATA4, ATG4A, HIDT1H2BN, THRB and MGST2, as compared to DNA methylation, is observed in non-cancer cells, and/or DNA hypomethylation of one or more of CACYBP, HIST1H2AJ, C1orf158, A4GALT, MLN, HIST1H3C, STC2 and ENG, as compared to DNA methylation, is observed in non-cancer cells, indicates a poor prognosis.

4. The method of claim 1, wherein the gene with DNA hypermethylation is ATG4A, HIST1H2BN, ADRA1A, CACNB2, GATA4, KCNA6, POU4F2, HS3ST2 or NEFH or any combination thereof.

5. The method of claim 1, wherein the gene with DNA hypermethylation is ATG4A, HIST1H2BN, CEACAM4, GATA4 or IGSF21 or any combination thereof.

6. The method of claim 1, wherein the gene with DNA hypermethylation is CEACAM4, GATA4 or IGSF21 or any combination thereof.

7. The method of claim 1, wherein the gene with DNA hypermethylation is POU4F2, NEFH, HS3ST2 or any combination thereof.

8. The method of claim 1, wherein the gene with DNA hypomethylation is CACYBP, or C1orf158 or a combination thereof.

9. The method of claim 1, wherein the gene with DNA hypomethylation 5 is CACYBP, or MLN or a combination thereof.

10. A method of making a treatment decision for a subject with ovarian cancer, comprising administering an effective amount of a demethylating agent to the subject, wherein the subject exhibits DNA hypermethylation of one or more of NPTX2, TNNI1, POU4F2, HS3ST2, CACNB2, TBX20, OR2L13, IGSF21, CD248, ADRA1A, NEFH, BNIP3, C1QTNF3, KCNA6, CEACAM4, CRNN, HFE2, TWIST1, GATA4, ATG4A, HIDT1H2BN, THRB and MGST2, or a polynucleotide sequence with at least 80% similarity thereof, as compared to DNA methylation observed in non-cancer cells.

11. The method of claim 10, wherein the demethylating agents is 5-aza-2′-deoxycytidine, 5-aza-cytidine, Zebularine, procaine, or L-ethionine.

12. The method of claim 10, wherein the gene with DNA hypermethylation is ATG4A, HIST1H2BN, CEACAM4, GATA4, NPTX2, TNNI1, POU4F2, HS3ST2, CACNB2, TBX20, OR2L13, IGSF21, CD248, ADRA1A, NEFH, BNIP3, C1QTNF3 or KCNA6 or any combination thereof.

13. The method of claim 10, wherein the gene with DNA hypermethylation is ATG4A, HIST1H2BN, ADRA1A, CACNB2, GATA4, KCNA6, POU4F2, HS3ST2 or NEFH or any combination thereof.

14. (canceled)

15. (canceled)

16. (canceled)

17. A method of determining a therapeutic regimen for a subject having a poor prognosis or malignancy in ovarian cancer, comprising providing chemotherapy to the subject, wherein the subject has DNA hypermethylation of one or more of NPTX2, TNNI1, POU4F2, HS3ST2, CACNB2, TBX20, OR2L13, IGSF21, CD248, ADRA1A, NEFH, BNIP3, C1QTNF3, KCNA6, CEACAM4, CRNN, HFE2, TWIST1, GATA4, ATG4A, HIDT1H2BN, THRB and MGST2, or a polynucleotide sequence with at least 80% similarity thereof, as compared to DNA methylation observed in non-cancer cells, and/or DNA hypomethylation of one or more of CACYBP, HIST1H2AJ, C1orf158, A4GALT, MLN, HIST1H3C, STC2 and ENG, as compared to DNA methylation observed in non-cancer cells.

18. The method of claim 17, wherein the gene with DNA hypermethylation is CEACAM4, GATA4, NPTX2, TNNI1, POU4F2, HS3ST2, CACNB2, TBX20, OR2L13, IGSF21, CD248, ADRA1A, NEFH, BNIP3, C1QTNF3 or KCNA6 or any combination thereof.

19. The method of claim 17, wherein the gene with DNA hypermethylation is ATG4A, HIST1H2BN, ADRA1A, CACNB2, GATA4, KCNA6, POU4F2, HS3ST2 or NEFH or any combination thereof.

20. The method of claim 17, wherein the gene with DNA hypermethylation is CEACAM4, GATA4 or IGSF21 or any combination thereof.

21. The method of claim 17, wherein the gene with DNA hypermethylation is POU4F2, NEFH, HS3ST2 or any combination thereof.

22. (canceled)

23. The method of claim 17, wherein the gene with DNA hypomethylation is CACYBP or C1orf158 or any combination thereof.

24. The method of claim 17, wherein the gene with DNA hypomethylation is CACYBP, or MLN or a combination thereof.

25. The method of claim 17, wherein the chemotherapy is adjuvant chemotherapy.

26. A kit for predicting risk or susceptibility of ovarian neoplasms or a prognosis, detecting malignancy and/or making a treatment decision for a subject with ovarian cancer, comprises reagents for differentiating methylated and non-methylated cytosine residues of one or more of the genes NPTX2, TNNI1, POU4F2, HS3ST2, CACNB2, TBX20, OR2L13, IGSF21, CD248, ADRA1A, NEFH, BNIP3, C1QTNF3, KCNA6, CEACAM4, CRNN, HFE2, TWIST1, GATA4, CACYBP, HIST1H2AJ, C1orf158, A4GALT, MLN, HIST1H3C, STC2, ATG4A, ENG, HIST1H2BN, MGST2 and THRB, or a polynucleotide sequence with at least 80% similarity thereof; wherein DNA hypermethylation of one or more of NPTX2, TNNI1, POU4F2, HS3ST2, CACNB2, TBX20, OR2L13, IGSF21, CD248, ADRA1A, NEFH, BNIP3, C1QTNF3, KCNA6, CEACAM4, CRNN, HFE2, TWIST1, GATA4, ATG4A, HIDT1H2BN, THRB and MGST2, as compared to DNA methylation observed in non-cancer cells, and/or DNA hypomethylation of one or more of CACYBP, HIST1H2AJ, C1orf158, A4GALT, MLN, HIST1H3C, STC2 and ENG, as compared to DNA methylation observed in non-cancer cells, indicates a poor prognosis or malignancy in ovarian cancer.

27. (canceled)

28. (canceled)
